(19) United States
(12) Patent Application Publication
(10) Pub. No.: US 2024/0376023 A1
(43) Pub. Date: Nov. 14, 2024

(54) METHOD FOR GENERATING A HYDROGEL FROM A CO2 GAS STREAM

(71) Applicant: SHELL USA, INC., Houston, TX (US)

(72) Inventors: Nicoleta Cristina NENU, Amsterdam (NL); Jana VAN DEN BERG, Amsterdam (NL); Christian DAVIES, Houston, TX (US); Andreas KLEMT, Leuna (DE)

(21) Appl. No.: 18/686,646

(22) PCT Filed: Sep. 20, 2022
(86) PCT No.: PCT/EP2022/076047

§ 371 Date: Feb. 26, 2024

(30) Foreign Application Priority Data
Sep. 29, 2021 (EP) 21199935.4

(51) Int. Cl. C05G 3/80; A01G 18/20; A01G 24/40; B01J 20/26; B01J 20/28; B01J 20/30; C05G 5/18; C08G 63/20; C09K 17/16; C09K 17/18

(52) U.S. Cl. CPC C05G 3/80; A01G 18/20; A01G 24/40; B01J 20/262; B01J 20/28047; B01J 20/3085; C05G 5/18; C08G 63/20; C09K 17/16; C09K 17/18

(57) ABSTRACT

The present disclosure relates to a method of sequestering carbon dioxide which comprises the steps of capturing carbon dioxide from an industrial gaseous waste stream and/or the atmosphere, converting a CO₂ from the CO₂ gas stream into a (COOH)₂ and combining the (COOH)₂, a mono-alcohol (X-OH), preferably CH₃CH₂OH, and a first acid catalyst comprising a H₂SO₄ at a temperature ranging from about 80° C. to about 100° C. and under atmospheric pressure to produce an ester comprising a (COOX)₂ and preferably (COOEt)₂; and the ester obtained is reacted with a polyol, preferably glycerine to form a polyester, preferably the polyester is a hydrogel. The present disclosure further relates to the use of a hydrogel which is obtainable by said method.

### FIELD OF THE DISCLOSURE

The present disclosure relates, in some embodiments, to systems and methods that convert CO₂ from a CO₂ stream (e.g., produced by an oil and gas industry asset) into a value product including a polymer in the form of a hydrogel and to polyesters obtained by said methods and agricultural compositions containing the polyesters.

### BACKGROUND OF THE DISCLOSURE

Our lives depend on energy as a major contributor for our daily survival. A large portion of the energy we use is derived from the processing and combustion of fossil fuels (e.g., hydrocarbon fuels). However, besides converting chemical energy to thermal and/or electrical energy, these combustion processes also generate greenhouse gases that are believed to be a leading cause of global climate change.

Two main tactics are used to deal with anthropogenic emissions of greenhouse gases. The first tactic is to simply find ways to reduce the overall fossil fuel consumption through energy use limitation or use of alternative energy methods. However, since energy is necessary, this tactic does little to deal with the greenhouse gases that will still be generated. A second option is to instead substantially transform the generated greenhouse gases into environmentally benign or even beneficial products.

Carbon dioxide (CO₂) is the primary greenhouse gas. There are currently existing technologies that capture and store CO₂ as it is generated from a fossil fuel processing or combustion system, but these technologies do not lead to the generation of beneficial products. There is a need for viable CO₂ gas abatement technologies that not only substantially capture the CO₂ once it is formed but then subsequently transform it into a useful commercially and environmentally beneficial products.

Additionally, besides adding to global climate change, the escape of terrestrial carbon as the backbone of CO₂ represents a lost opportunity to maintain adequate carbon levels in soil to promote crop growth. Therefore, not only is the loss of CO₂ damaging the atmosphere, but it is reducing access to a necessary soil resource. There is also a need to develop methods in which to increase soil carbon and protect the soil against loss of soil carbon. The two needs described above are not mutually exclusive and may be solved in tandem.

### SUMMARY OF THE INVENTION

Accordingly, there is a need for improved methods and systems for forming a polyester, in particular a hydrogel, from a CO₂ gas stream. Accordingly, there is also a need for improved products for use in agriculture which overcome the above identified problems. One or more of the above identified technical problems are solved by the teaching of the present invention, in particular the independent and dependent claims as well as the present description.

One or more of the above identified technical problems is solved by a method of sequestering carbon dioxide which comprises the steps of:
(a) capturing carbon dioxide from an industrial gaseous waste stream and/or the atmosphere;
(b) converting a CO₂ from the CO₂ gas stream into a (COOH)₂; and
(c) combining the (COOH)₂, a mono-alcohol (X-OH), preferably CH₃CH₂OH, and a first acid catalyst comprising a H₂SO₄ at a temperature ranging from about 80° C. to about 100° C. and under atmospheric pressure to produce an ester comprising a (COOX)₂ and preferably (COOEt)₂;
(d) the ester obtained in step (c) is reacted with a polyol, preferably glycerine to form a polyester, preferably the polyester is a hydrogel.

For the mono-alcohol X-OH, X stands for an alkyl group having the general formula: CₙH₂ₙ₊₁. The technical problem is further solved by a use of a hydrogel or a mixture comprising said hydrogel for sequestering carbon dioxide by promoting growth of fungi and increasing crop yield in agriculture and/or horticulture.

The technical problem is also solved by a polyester, in particular hydrogel, which is obtainable by the present method, agricultural compositions containing said polyester, in particular hydrogel, and uses and methods employing said polyester, in particular hydrogel, in agriculture.

### BRIEF DESCRIPTION OF THE DRAWINGS

Some embodiments of the disclosure may be understood by referring, in part, to the present disclosure and the accompanying drawings, wherein:

FIG. 1 is a diagram of a system for generating a hydrogel from a CO₂ gas stream.

FIG. 2 is a diagram of a system for generating a hydrogel from a CO₂ gas stream.

FIG. 3 is a plot showing water uptake and release cycles over time.

FIG. 4 is a plot showing a water uptake cycle over time.

FIG. 5 is a diagram showing the time dependence of the mass difference for samples of hydrogel.

FIG. 6 is a plot showing the time dependence of the mass difference for polyester samples.

FIG. 7 are plots showing the time dependence of the mass difference for polyester samples.

FIG. 8 is a plot showing the time dependence of the mass difference for a polyester sample.

FIG. 9 are plots showing the time dependence of the mass difference for a sample.

FIG. 10 are plots showing the time dependence of the mass difference of water uptake/release cycles for a sample at alternating temperatures.

FIG. 11 is a plot showing the enlarged section of the water uptake/release cycle of a sample.

FIG. 12 is a diagram showing the WHC comparison of samples.

FIG. 13 is a diagram comparing the water holding capacity (WHC) with commercially available hydrogels and Bentonite.

FIG. 14 is a diagram comparing the alpha diversity of the microbiome of soil only and samples of soil containing polyesters according to the present invention.

FIG. 15 is a diagram comparing the beta diversity of different samples.

FIG. 16 is a diagram showing the fungal to bacteria ratio of the microbiome of soil only and samples with different ingredients.

FIG. 17 are diagrams showing the weekly soil respiration assays of soil only and with additional ingredients.

FIG. 18 is a diagram showing the cumulative gel biodegradation rates of samples of soil including samples containing polyesters according to the present invention.

FIG. 19 is a diagram schematically showing a process for generating a polyester, in particular a hydrogel, according to the present invention.

FIG. 20 is a diagram showing the hydrostability of polyester samples according to the present invention.

### DETAILED DESCRIPTION

The present disclosure relates, in some embodiments, to systems and methods for generating a hydrogel from a CO₂ gas stream. A disclosed system and method advantageously and directly converts CO₂, a greenhouse gas, into a hydrogel, which may be used to increase and protect existing soil carbon levels. Additionally, disclosed systems and methods do this in a scalable and less complex manner in comparison to existing chemical synthetic technologies that also create additional environmentally harmful waste products. Additionally, many of the method steps and system components involve catalytic cycles that recycle byproducts from other methods steps and system components, thereby minimizing waste-products.

The invention provides for a process comprising a step (b) a gas stream comprising or consisting of CO₂ and converting the CO₂ into (COOH)₂ and in a second process step (c) converting the (COOH)₂ obtained in step (b) into a polyester, preferably the polyester is a hydrogel. Thus, the invention provides the teaching according to which from CO₂ contained in a gas stream or forming a gas stream, a polyester, in particular a branched or a linear polyester, in particular a branched polyester, is formed, which polyester is preferably a hydrogel and wherein said process has as an intermediate process step to produce from CO₂ the intermediate (COOH)₂ and converting the (COOH)₂ into the polyester.

In an embodiment of the present invention, step (c) of the process requires converting the (COOH)₂ into an ester and converting the ester into the polyester.

In an embodiment, step (c) of the method according to the present invention comprises combining the (COOH)₂ with a mono-alcohol to produce an ester and converting the ester into the polyester, in particular the hydrogel.

In a further preferred embodiment, step (c) of the method according to the present invention comprises passing the (COOH)₂ and a mono-alcohol, in particular methanol or ethanol, through an activated carbon bed to produce an ester, in particular a (COOMe)₂ or a (COOEt)₂.

In an embodiment, the mono-alcohol is selected from the group consisting of CH₃OH, CH₃CH₂OH, propanol, a straight alcohol consisting of 3 to 10 carbon atoms, a branched alcohol consisting of 3 to 10 carbon atoms and combinations thereof. In an embodiment, the mono-alcohol is CH₃OH or CH₃CH₂OH. In a furthermore preferred embodiment, the ester produced is (COOMe)₂ in case the mono-alcohol is CH₃OH or (COOEt)₂ in case the mono-alcohol is

CH₃CH₂OH.

In a further preferred embodiment, step (c) of the method according to the present invention comprises passing the (COOH)₂ through an activated carbon bed to produce a (COOH)₂ absorbed carbon bed, in particular a (COOH)₂ absorbed carbon bed saturated with (COOH)₂, in particular with 1 to 100%, in particular 10 to 100%, in particular 20 to 80%, in particular 40 to 60%, in particular 1%, in particular 10%, in particular 20%, in particular 30%, in particular 40%, in particular 50%, in particular 60%, in particular 70%, in particular 80%, in particular 90%, in particular 100% (COOH)₂.

In a further preferred embodiment, step (c) of the method according to the present invention comprises passing a mono-alcohol, in particular methanol or ethanol, through a (COOH)₂ absorbed carbon bed to produce an ester, in particular a (COOMe)₂ or a (COOEt)₂.

In an embodiment, in step (c) of the method according to the present invention the (COOH)₂ is combined with the mono-alcohol to produce an ester and the ester is converted into the polyester, in particular the hydrogel, said combining is conducted in the presence of a first acid catalyst. The first acid catalyst catalyses the conversion of the (COOH)₂ and the mono-alcohol to a (COOH)₂ ester, which is also abbreviated to be an ester.

In an embodiment of the present invention, the first acid catalyst is selected from the group consisting of H₂SO₄, hydrochloric acid, nitric acid, hydrobromic acid, hydroiodic acid, perchloric acid, chloric acid, acetic acid, sulfurous acid, methanoic acid, phosphoric acid, nitrous acid, hydrofluoric acid, and combinations thereof.

In an embodiment, the first acid catalyst is H₂SO₄.

In an embodiment of the present invention, step (c) of the present invention is conducted using a first acid catalyst, in particular 0.001 to 5.0 wt. % of a first acid catalyst, in particular 0.01 to 1.125 wt. %, in particular 0.05 to 0.8 wt. %, in particular 0.1 to 0.6 wt. %, in particular 0.2 to 0.5 wt. %, in particular 0.3 to 0.4 wt. %, in particular 0.01 wt. %, in particular 0.05 wt. %, in particular 0.1 wt. %, in particular 0.25 wt. %, in particular 0.5 wt. %, in particular 0.75 wt. %, in particular 1.0 wt. %, in particular 1.125 wt. % in particular 1.25 wt. % (each in relation to the amount of (COOH)₂).

In an embodiment, combining the (COOH)₂ with the mono-alcohol to produce an ester is conducted under atmospheric pressure.

In an embodiment, combining the (COOH)₂ with the mono-alcohol to produce an ester is conducted in a distillation reactor.

In an embodiment, the method according to the present invention in step (c) further comprises:
combining the (COOH)₂ with a mono-alcohol, in particular CH₃OH or CH₃CH₂OH, to produce an ester, in particular, wherein the ester is a (COOMe)₂ or a (COOEt)₂, preferably in the presence of a first acid catalyst, in particular an acid catalyst comprising a H₂SO₄, preferably at a temperature from 80° C. to 100° C. and preferably under atmospheric pressure.

In an embodiment of the present invention, combining the (COOH)₂ with the mono-alcohol to produce an ester is conducted in the presence of a solvent, in particular an organic or inorganic solvent, in particular a polar organic solvent or a polar inorganic solvent, in particular CH₃OH, CH₃CH₂OH, propanol, a straight alcohol consisting of 3 to 10 carbon atoms, a branched alcohol consisting of 3 to 10 carbon atoms, water or combinations thereof.

In an embodiment of the present invention, combining the (COOH)₂ with the mono-alcohol to produce an ester is conducted in the presence of a solvent, wherein the solvent is the mono-alcohol.

In an embodiment of the present invention, combining the (COOH)₂ with the mono-alcohol, in particular CH₃OH, CH₃CH₂OH, propanol, a straight alcohol consisting of 3 to 10 carbon atoms, a branched alcohol consisting of 3 to 10 carbon atoms or combinations thereof, to produce an ester is conducted in the presence of a solvent, wherein the solvent is the mono-alcohol, in particular CH₃OH, CH₃CH₂OH, propanol, a straight alcohol consisting of 3 to 10 carbon atoms, a branched alcohol consisting of 3 to 10 carbon atoms or combinations thereof.

In an embodiment of the present invention, in step (d) of the method according to the present invention, the converting of the ester into a polyester comprises:
combining the ester with the polyol to produce the polyester.

In an embodiment of the present invention, the ester is combined with the polyol, which in an embodiment is glycerine, to produce the polyester, in particular wherein the reaction of the ester with the polyol produces the polyester and a mono-alcohol.

In an embodiment of the present invention, combining the ester with the polyol to produce the polyester is conducted in the presence of a solvent, in particular an organic or inorganic solvent, in particular a polar organic solvent or a polar inorganic solvent, in particular water or the mono-alcohol used to produce the ester, in particular CH₃OH, CH₃CH₂OH, propanol, a straight alcohol consisting of 3 to 10 carbon atoms, a branched alcohol consisting of 3 to 10 carbon atoms or combinations thereof. In an embodiment, the process step (d) is conducted in the presence of at least one second catalyst, in particular second acid catalyst. The second acid catalyst catalyses the conversion of the ester and the polyol to a polyester and optionally water or a mono-alcohol.

In an embodiment of the present invention, the second acid catalyst is a Brønsted acid. In a further preferred embodiment of the present invention, the second acid catalyst is a Lewis acid.

In a further preferred embodiment of the present invention, the second acid catalyst is a Brønsted acid and/or a Lewis acid.

Preferably, the second acid catalyst can be H₂SO₄, Sb₂O₃, SnCl₂, titanium isopropoxide or p-toluenesulfonic acid.

In an embodiment of the present invention, step (d) of the present invention is conducted with a second acid catalyst, in particular with 0.001 to 5.0 wt. % of a second acid catalyst, in particular 0.01 to 1.125 wt. %, in particular 0.01 to 1.0 wt. %, in particular 0.05 to 0.8 wt. %, in particular 0.1 to 0.6 wt. %, in particular 0.2 to 0.5 wt. %, in particular 0.3 to 0.4 wt. %, in particular 0.01 wt. %, in particular 0.05 wt. %, in particular 0.1 wt. %, in particular 0.25 wt. %, in particular 0.5 wt. %, in particular 0.75 wt. %, in particular 1.0 wt. %, in particular 1.125 wt. % in particular 1.25 wt. %, in particular 5.0 wt. % (each in relation to the amount of ester plus the amount of polyol).

In an embodiment of the present invention, step (d) of the present invention is conducted with a second acid catalyst, in particular with 0.001 to 8.0 mol % of a second acid catalyst, in particular 0.01 to 8.0 mol %, in particular 0.05 to 0.8 mol %, in particular 0.1 to 0.6 mol %, in particular 0.2 to 0.5 mol %, in particular 0.3 to 0.4 mol %, in particular 0.001, in particular 0.006, in particular 0.01 mol %, in particular in particular 0.05 mol %, in particular 0.1 mol %, in particular 0.25 mol %, in particular 0.5 mol %, in particular 0.75 mol %, in particular 1.0 mol %, in particular 1.18 mol %, in particular 1.25 mol %, in particular 5.0 mol %, in particular 8 mol % (each in relation to the amount of (COOH)₂ or ester plus the amount of polyol).

In an embodiment, in step (d) of the method according to the present invention the converting of the (COOH)₂ or the ester into a polyester comprises:
combining the (COOH)₂ or the ester with a polyol, in particular glycerine, to produce the polyester and optionally water or a mono-alcohol, in particular a hydrogel and optionally water or a mono-alcohol, preferably in the presence of a second acid catalyst, in particular a second catalyst comprising a H₂SO₄, Sb₂O₃, SnCl₂, titanium isopropoxide or p-toluenesulfonic acid.

In an embodiment, in step (d) of the method according to the present invention the convertion of the ester into a polyester comprises:
combining the ester with a polyol, in particular glycerine, to produce the polyester and optionally water or a mono-alcohol, in particular a hydrogel and optionally water or a mono-alcohol, preferably in the presence of a second acid catalyst, in particular 0.001 to 8.0 mol % of a second acid catalyst, in particular 0.01 to 8.0 mol %, in particular 0.05 to 0.8 mol %, in particular 0.1 to 0.6 mol %, in particular 0.2 to 0.5 mol %, in particular 0.3 to 0.4 mol %, in particular 0.001, in particular 0.006, in particular 0.01 mol %, in particular 0.05 mol %, in particular 0.1 mol %, in particular 0.25 mol %, in particular 0.5 mol %, in particular 0.75 mol %, in particular 1.0 mol %, in particular 1.18 mol %, in particular 1.25 mol %, in particular 5.0 mol %, in particular 8 mol % (each in relation to the amount of ester plus the amount of polyol), in particular a second acid catalyst comprising a H₂SO₄, Sb₂O₃, SnCl₂, titanium isopropoxide or p-toluenesulfonic acid.

In an embodiment of the present invention, step (c) and/or (d) of the present method, is conducted in a polymerization reactor.

In an embodiment of the present invention, step (d) of the present method, in particular the converting of the ester into a polyester, is conducted at a temperature from 80 to 205° C., preferably 100 to 180° C., preferably 100 to 170° C., of preferably 95° C., preferably 100° C., preferably 110° C., preferably 120° C., preferably 130° C., preferably 140° C., preferably 150° C., preferably 160° C., preferably 170° C., preferably 180° C., preferably 190° C., preferably 200° C., preferably 205° C.

In an embodiment of the present invention, the step (d) of the present method, in particular the converting of the ester into a polyester, is conducted at a pressure from 0.001 to 105 bara, in particular 0.01 to 1 bara, in particular 0.02 to 1 bara, preferably 0.3 to 1 bara, preferably 0.4 to 1 bara, preferably 0.5 to 1 bara, of in particular 0.001 bara, in particular 0.01 bara, in particular 0.02 bara, in particular 0.1 bara, in particular 0.3 bara, in particular 0.4 bara, in particular 0.5 bara, in particular 0.6 bara, in particular 0.7 bara, in particular 0.8 bara, in particular 0.9 bara, in particular 1.0 bara.

In an embodiment, in step (d) of the method according to the present invention the converting of the ester into a polyester comprises:
combining the ester, in particular wherein the ester is (COOMe)₂ or (COOEt)₂, with a polyol, in particular glycerine, in the presence of a second acid catalyst, in particular a second acid catalyst comprising a H₂SO₄, Sb₂O₃, SnCl₂, titanium isopropoxide or p-toluenesulfonic acid, at a temperature from 100 to 180° C., preferably 100 to 170° C., and a pressure from 0.02 bara to 1 bara to produce the polyester, in particular the hydrogel, and optionally water or a mono-alcohol, in particular a methanol or an ethanol.

In an embodiment, in step (d) of the method according to the present invention the combining of the ester, in particular wherein the ester is (COOMe)₂ or (COOEt)₂, with a polyol to produce the polyester, in particular the hydrogel, and optionally water or a mono-alcohol, in particular a methanol or an ethanol, is conducted at a temperature from 100 to 180° C., preferably 100 to 170° C., in particular 110 to 160° C., in particular 120 to 150° C., of in particular 100° C., in particular 110° C., in particular 120° C., in particular 130° C., in particular 140° C., in particular 150° C., in particular 160° C., in particular 170° C., and a pressure from 0.001 to 105 bara, in particular 0.01 to 1 bara, in particular 0.02 to 1 bara, preferably 0.3 to 1 bara, preferably 0.4 to 1 bara, preferably 0.5 to 1 bara, of in particular 0.001 bara, in particular 0.01 bara, in particular 0.02 bara, in particular 0.1 bara, in particular 0.3 bara, in particular 0.4 bara, in particular 0.5 bara, in particular 0.6 bara, in particular 0.7 bara, in particular 0.8 bara, in particular 0.9 bara, in particular 1.0 bara. In an embodiment, in step (d) of the method according to the present invention the combining the (COOH)₂ or the ester, in particular wherein the ester is (COOMe)₂ or (COOEt)₂, with a polyol to produce the polyester, in particular the hydrogel, and optionally water or a mono-alcohol, in particular a methanol or an ethanol, is conducted preferably in the presence of a second acid catalyst, in particular 0.001 to 8.0 mol % of a second acid catalyst, in particular 0.01 to 8.0 mol %, in particular 0.05 to 0.8 mol %, in particular 0.1 to 0.6 mol %, in particular 0.2 to 0.5 mol %, in particular 0.3 to 0.4 mol %, in particular 0.001, in particular 0.006, in particular 0.01 mol %, in particular 0.05 mol %, in particular 0.1 mol %, in particular 0.25 mol %, in particular 0.5 mol %, in particular 0.75 mol %, in particular 1.0 mol %, in particular 1.18 mol %, in particular 1.25 mol %, in particular 5.0 mol %, in particular 8 mol % (each in relation to the amount of (COOH)₂ or ester plus the amount of polyol), in particular a second acid catalyst comprising a H₂SO₄, Sb₂O₃, SnCl₂, titanium isopropoxide or p-toluenesulfonic acid, at a temperature from 100 to 180° C., preferably 100 to 170° C., in particular 110 to 160° C., in particular 120 to 150° C., in particular 100° C., in particular 110° C., in particular 120° C., in particular 130° C., in particular 140° C., in particular 150° C., in particular 160° C., in particular 170° C., and a pressure from 0.001 to 105 bara, in particular 0.01 to 1 bara, in particular 0.02 to 1 bara, in particular 0.1 bara, preferably 0.3 to 1 bara, preferably 0.4 to 1 bara, preferably 0.5 to 1 bara, of in particular 0.001 bara, in particular 0.01 bara, in particular 0.02 bara, in particular 0.1 bara, in particular 0.3 bara, in particular 0.4 bara, in particular 0.5 bara, in particular 0.6 bara, in particular 0.7 bara, in particular 0.8 bara, in particular 0.9 bara, in particular 1.0 bara. In an embodiment, the polyol used in the method according to the present invention is glycerine and the polyester is composed, in particular consists, of monomeric building units of (COOH)₂ and glycerine, preferably wherein the polyester, in particular the hydrogel, has a structure according to Formula I (this reference will be adhered to in the present disclosure):

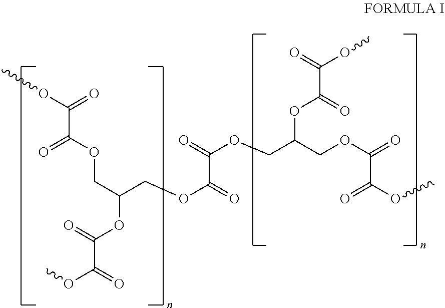

with n ranging from 1 to 5250, preferably 1 to 5000.

In an embodiment, n is ranging from 500 to 4500, preferably 1000 to 4000, preferably 1500 to 3500 or preferably 2000 to 3000.

In an embodiment of the present invention n is 1. In an embodiment of the present invention n is ranging from 250 to 750. In an embodiment of the present invention n is ranging from 750 to 1250. In an embodiment of the present invention n is ranging from 1250 to 1750. In an embodiment of the present invention n is ranging from 1750 to 2250. In an embodiment of the present invention n is ranging from 2250 to 2750. In an embodiment of the present invention n is ranging from 2750 to 3250. In an embodiment of the present invention n is ranging from 3250 to 3750. In an embodiment of the present invention n is ranging from 3750 to 4250. In an embodiment of the present invention n is ranging from 4250 to 4750. In an embodiment of the present invention n is ranging from 4750 to 5250.

In an embodiment, the polyol used in the method according to the present invention is glycerine and the polyester is composed, in particular consists, of monomeric building units of (COOH)₂ and glycerine, preferably wherein the polyester, in particular the hydrogel, has a structure according to Formula II, III, or IV (these references will be adhered to in the present disclosure):

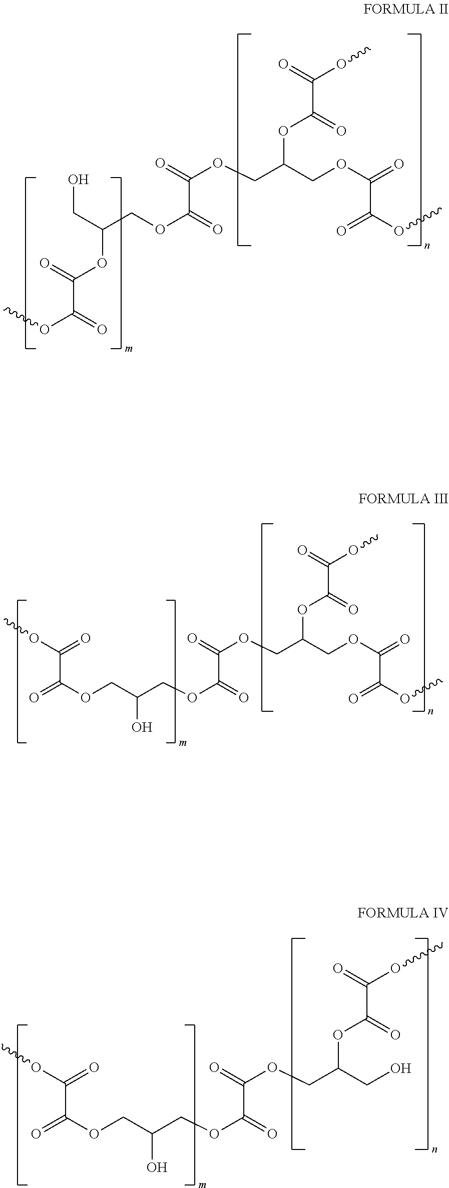

with n and m each ranging independently from 1 to 5250, preferably 1 to 5000.

In an embodiment, m is ranging from 500 to 4500, preferably 1000 to 4000, preferably 1500 to 3500 or preferably 2000 to 3000.

In an embodiment of the present invention m is 1. In an embodiment of the present invention m is ranging from 250 to 750. In an embodiment of the present invention m is ranging from 750 to 1250. In an embodiment of the present invention m is ranging from 1250 to 1750. In an embodiment of the present invention m is ranging from 1750 to 2250. In an embodiment of the present invention m is ranging from 2250 to 2750. In an embodiment of the present invention m is ranging from 2750 to 3250. In an embodiment of the present invention m is ranging from 3250 to 3750. In an embodiment of the present invention m is ranging from 3750 to 4250. In an embodiment of the present invention m is ranging from 4250 to 4750. In an embodiment of the present invention m is ranging from 4750 to 5250.

In an embodiment, n and m are each ranging independently from 500 to 4500, preferably 1000 to 4000, preferably 1500 to 3500 or preferably 2000 to 3000.

In an embodiment of the present invention n and m are 1. In an embodiment of the present invention n and m are each ranging independently from 250 to 750. In an embodiment of the present invention n and m are each ranging independently from 750 to 1250. In an embodiment of the present invention n and m are each ranging independently from 1250 to 1750. In an embodiment of the present invention n and m are each ranging independently from 1750 to 2250. In an embodiment of the present invention n and m are each ranging independently from 2250 to 2750. In an embodiment of the present invention n and m are each ranging independently from 2750 to 3250. In an embodiment of the present invention n and m are each ranging independently from 3250 to 3750. In an embodiment of the present invention n and m are each ranging independently from 3750 to 4250. In an embodiment of the present invention n and m are each ranging independently from 4250 to 4750. In an embodiment of the present invention n and m are each ranging independently from 4750 to 5250.

In an embodiment, the polyol used in the method according to the present invention is glycerine and the polyester is composed, in particular consists, of monomeric building units of (COOH)₂ and glycerine, preferably wherein the polyester, in particular the hydrogel, has a structure according to Formula I, II, III, or IV, with n and/or m each ranging independently from 1 to 5250, preferably 1 to 5000.

In an embodiment, the polyol used in the method according to the present invention is glycerine and the polyester is composed, in particular consists, of monomeric building units of (COOH)₂ and glycerine, preferably wherein the polyester, in particular the hydrogel, has a structure according to Formula I with n ranging from 1 to 5250, preferably 1 to 5000 and a weight average molecular weight of 400 to 50000 g/mol, in particular 1000 to 50000 g/mol, in particular 1000 to 30000 g/mol, in particular 10000 to 20000 g/mol, in particular 1000 g/mol, in particular 5000 g/mol, in particular 10000 g/mol, in particular 11789 g/mol, in particular 14420 g/mol, in particular 15000 g/mol, in particular 20000 g/mol, in particular 30000 g/mol, in particular 50000 g/mol, measured by GPC and/or ESI-TOF-MS.

In an embodiment, the polyol used in the method according to the present invention is glycerine and the polyester is composed, in particular consists, of monomeric building units of (COOH)₂ and glycerine, preferably wherein the polyester, in particular the hydrogel, has a structure according to Formula II, III or IV with n and m each ranging independently from 1 to 5250, preferably 1 to 5000 and a weight average molecular weight of 400 to 50000 g/mol, in particular 1000 to 50000 g/mol, in particular 1000 to 30000 g/mol, in particular 10000 to 20000 g/mol, in particular 1000 g/mol, in particular 5000 g/mol, in particular 10000 g/mol, in particular 11789 g/mol, in particular 14420 g/mol, in particular 15000 g/mol, in particular 20000 g/mol, in particular 30000 g/mol, in particular 50000 g/mol, measured by GPC and/or ESI-TOF-MS.

In an embodiment, the polyol used in the method according to the present invention is glycerine and the polyester is composed, in particular consists, of monomeric building units of (COOH)₂ and glycerine, preferably wherein the polyester, in particular the hydrogel, has a structure according to Formula I, II, III or IV with n and/or m each ranging independently from 1 to 5250, preferably 1 to 5000 and a weight average molecular weight of 400 to 50000 g/mol, in particular 1000 to 50000 g/mol, in particular 1000 to 30000 g/mol, in particular 10000 to 20000 g/mol, in particular 1000 g/mol, in particular 5000 g/mol, in particular 10000 g/mol, in particular 11789 g/mol, in particular 14420 g/mol, in particular 15000 g/mol, in particular 20000 g/mol, in particular 30000 g/mol, in particular 50000 g/mol, measured by GPC and/or ESI-TOF-MS.

In an embodiment, in step (b) of the method according to the present invention converting the CO₂ into the (COOH)₂ comprises:
passing the CO₂, in particular the CO₂ gas stream, through a water bath to produce a carbonated water; and passing the carbonated water through a metal ion exchanger, in particular metal ion exchange bubble column, comprising a M₂(COO)₂ to produce the (COOH)₂ and a MHCO₃.

In an embodiment, the water bath in step (b) of the method according to the present invention is set to a temperature ranging from −5 to 105° C., in particular 10 to 100° C., in particular 20 to 80° C., in particular 30 to 60° C., in particular 40 to 50° C., of in particular −5° C., in particular 0° C., in particular 10° C., in particular 20° C., in particular 30° C., in particular 40° C., in particular 50° C., in particular 60° C., in particular 70° C., in particular 80° C., in particular 90° C., in particular 100°° C., in particular 105° C.

In an embodiment, the carbonated water produced in step (b) of the method according to the present invention is a water saturated with CO₂, in particular with 1 to 100% of CO₂, in particular 10 to 100% of CO₂, in particular 30 to 100% of CO₂, in particular 40 to 100% of CO₂, in particular 1% of CO₂, in particular 10% of CO₂, in particular 20% of CO₂, in particular 30% of CO₂, in particular 40% of CO₂, in particular 50% of CO₂, in particular 60% of CO₂, in particular 70% of CO₂, in particular 80% of CO₂, in particular 90% of CO₂, in particular 100% of CO₂.

In an embodiment, the metal ion exchanger in step (b) of the method according to the present invention is a metal ion exchange bubble column and/or ion exchange resins, in particular potassium or natrium ion exchange resins.

In an embodiment, the metal ion exchanger, in particular the metal ion exchange bubble column, is operated at a temperature from 2.5 to 62.5° C., preferably 5 to 60° C., preferably 10 to 50° C., preferably 15 to 40° C., preferably 20 to 35° C., of preferably 2.5° C., preferably 5° C., preferably 10° C., preferably 15° C., preferably 20° C., preferably 25° C., preferably 30° C., preferably 35° C., preferably 40° C., preferably 45° C., preferably 50° C., preferably 55° C., preferably 60° C. preferably 62.5° C.

In an embodiment, the metal ion exchanger, in particular the metal ion exchange bubble column, is operated at a temperature from 2.5 to 62.5° C., preferably 5 to 60° C., preferably 10 to 50° C., preferably 15 to 40° C., preferably 20 to 35° C., of preferably 2.5° C., preferably 5° C., preferably 10° C., preferably 15° C., preferably 20° C., preferably 25° C., preferably 30° C., preferably 35° C., preferably 40° C., preferably 45° C., preferably 50° C., preferably 55° C., preferably 60°° C. preferably 62.5° C. and a water saturated with CO₂, in particular with 1 to 100% of CO₂, in particular 10 to 100% of CO₂, in particular 30 to 100% of CO₂, in particular 40 to 100% of CO₂, in particular 1% of CO₂, in particular 10% of CO₂, in particular 20% of CO₂, in particular 30% of CO₂, in particular 40% of CO₂, in particular 50% of CO₂, in particular 60% of CO₂, in particular 70% of CO₂, in particular 80% of CO₂, in particular 90% of CO₂, in particular 100% of CO₂.

In an embodiment, the method according to the present invention further comprises, preferably subsequent to step (b):
(b1) combining the MHCO₃ produced from the metal ion exchanger, in particular the metal ion exchange bubble column, with a hydrogen gas, preferably in a hydrogenation reactor comprising a hydrogenation catalyst, to produce a mixture comprising a HCOOM and a MHCO₃. In an embodiment, the HCOOM is HCOOK or HCOONa.

In an embodiment of the present invention, the hydrogenation catalyst is at least one hydrogenation catalyst selected from the group consisting of a palladium catalyst, in particular Pd/C, Pd/Al₂O₃, Pd/theta Al₂O₃, a nickel catalyst, in particular Ni/SiO₂, a platinum catalyst, a rhodium catalyst, a ruthenium catalyst, a cobalt catalyst, a copperchromite catalyst (Adkins catalyst), a zincchromite catalyst an iron catalyst, a SiO₂/Al₂O₃ and combinations thereof.

In an embodiment of the present invention, the hydrogenation catalyst is at least one hydrogenation catalyst selected from the group consisting of Pd/C, Pd/Al₂O₃, Pd/theta Al₂O₃, Ni/SiO₂, SiO₂/Al₂O₃ and combinations thereof.

In an embodiment of the present invention, the hydrogenation catalyst is used in a concentration ranging from 0.075 g/100 mL to 5.25 g/100 mL of solvent used in the hydrogenation reaction, in particular 0.1 g/100 mL to 5.5 g/100 mL of solvent used in the hydrogenation reaction, in particular from 0.1 g/100 mL to 5.0 g/100 mL of solvent, in particular from 0.5 g/100 mL to 4.5 g/100 mL of solvent, in particular from 1.0 g/100 mL to 4.0 g/100 mL of solvent, in particular from 1.5 g/100 mL to 3.5 g/100 mL of solvent, in particular from 2.0 g/100 mL to 3.0 g/100 mL of solvent, of in particular 0.075 g/100 mL, in particular 0.1 g/100 mL of solvent, in particular 0.2 g/100 mL of solvent, in particular 0.5 g/100 mL of solvent, in particular 1.0 g/100 mL of solvent, in particular 1.5 g/100 mL of solvent, in particular 2.0 g/100 mL of solvent, in particular 2.5 g/100 mL of solvent, in particular 3.0 g/100 mL of solvent, in particular 3.5 g/100 mL of solvent, in particular 4.0 g/100 mL of solvent, in particular 4.5 g/100 mL of solvent, in particular 5.0 g/100 mL of solvent, in particular 5.25 g/100 mL of solvent used in the hydrogenation reaction.

In an embodiment of the present invention, the solvent used in the hydrogenation reaction or stirred-tank reactor is water.

In an embodiment of the present invention, the hydrogenation catalyst is used, preferably in a hydrogenation reactor or stirred-tank reactor, in a molar amount ranging from 0.01 to 50 mol %, in particular 0.1 to 20 mol %, in particular 0.1 to 10 mol %, in particular 0.5 to 10 mol %, in particular 0.5 to 5 mol %, in particular 1.0 to 5 mol %, in particular 1.0 to 2 mol %, of in particular 0.01 mol %, in particular 0. 1 mol %, in particular 0.2 mol %, in particular 0.5 mol %, in particular 1.0 mol %, in particular 2.0 mol %, in particular 5.0 mol %, in particular 10.0 mol %, in particular 20.0 mol %, in particular 50.0 mol % of the hydrogenation catalyst (each in relation to the amount of MHCO₃).

In an embodiment, the hydrogenation reactor or stirred-tank reactor is operated at a temperature from 12.5 to 152.5° C., preferably 15 to 150° C., preferably 20 to 100° C., preferably 30 to 75° C., preferably 40 to 60° C., of preferably 12.5° C., preferably 15° C., preferably 20°° C., preferably 25° C., preferably 30° C., preferably 35° C., preferably 40° C., preferably 45° C., preferably 50° C., preferably 55° C., preferably 60° C., preferably 65° C., preferably 70° C., preferably 75° C., preferably 80° C., preferably 85° C., preferably 90° C., preferably 95° C., preferably 100° C., preferably 105° C., preferably 110° C., preferably 115° C., preferably 120° C., preferably 125° C., preferably 130° C., preferably 135° C., preferably 140° C., preferably 145° C., preferably 150° C., preferably 152.5° C.

In an embodiment, the hydrogenation reactor or stirred-tank reactor is operated at a pressure ranging from 0.01 to 100 bara, preferably 0.1 to 80 bara, preferably 1.0 to 50 bara, preferably 10 to 50 bara, preferably 20 to 30 bara, of preferably 0.01 bara, preferably 0.1 bara, preferably 1.0 bara, preferably 10 bara, preferably 20 bara, preferably 30 bara, preferably 50 bara, preferably 80 bara, preferably 100 bara.

In an embodiment, the hydrogenation reactor or stirred-tank reactor is operated at a temperature ranging from 12.5 to 152.5° C., preferably 15 to 150° C., preferably 20 to 100° C., preferably 30 to 75° C., preferably 40 to 60° C., of preferably 12.5° C., preferably 15° C., preferably 20° C., preferably 25° C., preferably 30° C., preferably 35° C., preferably 40° C., preferably 45° C., preferably 50° C., preferably 55° C., preferably 60° C., preferably 65° C., preferably 70° C., preferably 75° C., preferably 80° C., preferably 85° C., preferably 90° C., preferably 95° C., preferably 100° C., preferably 105° C., preferably 110° C., preferably 115° C., preferably 120° C., preferably 125° C., preferably 130° C., preferably 135° C., preferably 140° C., preferably 145° C., preferably 150° C., preferably 152.5° C. and a pressure ranging from 0.01 to 100 bara, preferably 0.1 to 80 bara, preferably 1.0 to 50 bara, preferably 10 to 50 bara, preferably 20 to 30 bara, of preferably 0.01 bara, preferably 0.1 bara, preferably 1.0 bara, preferably 10 bara, preferably 20 bara, preferably 30 bara, preferably 50 bara, preferably 80 bara, preferably 100 bara.

In an embodiment of the present invention, the hydrogenation reactor or stirred-tank reactor contains 0.01 to 50 mol %, in particular 0.1 to 20 mol %, in particular 0.1 to 10 mol % of the hydrogenation catalyst (each in relation to the amount of MHCO₃) and the hydrogenation reactor is operated at a temperature ranging from 12.5 to 152.5° C., preferably 15 to 150° C., preferably 20 to 100° C., preferably 30 to 75° C., preferably 40 to 60° C., of preferably 12.5° C., preferably 15° C., preferably 20° C., preferably 25° C., preferably 30° C., preferably 35° C., preferably 40° C., preferably 45° C., preferably 50° C., preferably 55° C., preferably 60° C., preferably 65° C., preferably 70° C., preferably 75° C., preferably 80° C., preferably 85° C., preferably 90° C., preferably 95° C., preferably 100° C., preferably 105° C., preferably 110° C., preferably 115° C., preferably 120° C., preferably 125° C., preferably 130° C., preferably 135° C., preferably 140° C., preferably 145° C., preferably 150° C., preferably 152.5° C. and/or a pressure ranging from 0.01 to 100 bara, preferably 0.1 to 80 bara, preferably 1.0 to 50 bara, preferably 10 to 50 bara, preferably 20 to 30 bara, of preferably 0.01 bara, preferably 0.1 bara, preferably 1.0 bara, preferably 10 bara, preferably 20 bara, preferably 30 bara, preferably 50 bara, preferably 80 bara, preferably 100 bara.

In an embodiment of the present invention, the hydrogenation reactor or stirred-tank reactor contains a MHCO₃ at a concentration ranging from 0.1 to 10 mol/L of solvent used in the hydrogenation reaction, in particular 0.5 to 5.0 mol/L of solvent, in particular 1.0 to 5.0 mol/L of solvent, in particular 2.0 to 4.0 mol/L of solvent, of in particular 0.1 mol/L of solvent, 0.25 mol/L, in particular 0.5 mol/L of solvent, in particular 1.0 mol/L of solvent, in particular 2.0 mol/L of solvent, in particular 3.0 mol/L of solvent, in particular 4.0 mol/L of solvent, in particular 5.0 mol/L of solvent, in particular 5.25 mol/L, in particular 10 mol/L.

In an embodiment of the present invention, the hydrogenation reactor or stirred-tank reactor is operated at a liquid hourly space velocity (LHSV) ranging from 0.01 to 10 1/h, in particular from 0.1 to 5.0 1/h, in particular from 1.0 to 5.0 1/h, in particular from 2.0 to 4.0 1/h, in particular from 2.0 to 3.0 1/h, of in particular 0.01 1/h, in particular 0.1 1/h, in particular 1.0 1/h, in particular 2.0 1/h, in particular 3.0 1/h, in particular 4.0 1/h, in particular 5.0 1/h.

In an embodiment of the present invention, the hydrogenation reactor or stirred-tank reactor is operated at a hydrogen pressure ranging from 0.00075 bara to 105 bara, in particular 0.01 bara to 80 bara, in particular 0.1 bara to 50 bara, in particular 1 bara to 50 bara, in particular 10 bara to 40 bara, in particular 20 bara to 30 bara, of in particular 0.00075 bara, in particular 0.001 bara, in particular 0.005 bara in particular 0.01 bara, in particular 0.05 bara, in particular 0.1 bara, in particular 0.5 bara, in particular 1.0 bara, in particular 1.0025 bara, in particular 1.025 bara in particular 1.25 bara in particular 10 bara, in particular 20 bara, in particular 30 bara, in particular 40 bara, in particular 50 bara, in particular 60 bara, in particular 70 bara, in particular 80 bara, in particular 90 bara, in particular 100 bara, in particular 105.

In an embodiment, the method according to the present invention further comprises, preferably subsequent to step (b):
(b2) combining the MHCO₃ produced from the metal ion exchanger, in particular the metal ion exchange bubble column, with a hydrogen gas, preferably in a stirred-tank reactor, comprising a hydrogenation catalyst, to produce a mixture comprising a HCOOM and a MHCO₃. In an embodiment, the HCOOM is HCOOK or HCOONa.

In an embodiment of the present invention, the stirred-tank reactor contains a catalyst concentration, in particular a suspended catalyst concentration, ranging from 0.01 g/L to 105 g/L of solvent used in the hydrogenation reaction, in particular 0.01 g/L to 100 g/L, in particular 0.1 g/L to 80 g/L, in particular 1.0 g/L to 50 g/L, in particular 1.0 g/L to 30 g/L, in particular 10 g/L to 20 g/L, of in particular 0.01 g/L, in particular 0.06 g/L, in particular 0.1 g/L, in particular 0.15 g/L, in particular 1.0 g/L, in particular 6.0 g/L, in particular 10 g/L, in particular 20 g/L, in particular 30 g/L, in particular 40 g/L, in particular 50 g/L, in particular 60 g/L, in particular 70 g/L, in particular 80 g/L, in particular 90 g/L, in particular 100 g/L, in particular 105 g/L.

In an embodiment of the present invention, the method further comprises a step to separate the mixture produced in step (b), (b1) or (b2), preferably through fractional crystallization. In an embodiment, the separation of the mixture produced in step (b), (b1) or (b2) is conducted in a crystallization unit.

In an embodiment, the separation produces a MHCO₃ and a HCOOM, which are separated from each other.

In an embodiment, the method according to the present invention further comprises: separating the mixture produced in step (b), (b1) or (b2) preferably through fractional crystallization, preferably in a crystallization unit, into a separated MHCO₃ and a separated HCOOM.

In an embodiment, the mixture produced in step (b), (b1) or (b2) is separated through fractional crystallization, preferably in a crystallization unit, at a temperature ranging from −25 to 525° C., in particular 0 to 500° C., in particular 50 to 400° C., in particular 100 to 300° C., of in particular −25° C., in particular 0° C., in particular 10° C., in particular 20° C., in particular 30° C., in particular 50° C., in particular 100° C., in particular 150° C., in particular 200° C., in particular 250° C., in particular 300° C., in particular 350° C., in particular 400° C., in particular 450° C., in particular 500° C., in particular 525° C.

In an embodiment, the mixture produced in step (b), (b1) or (b2) is separated through fractional crystallization, preferably in a crystallization unit, wherein the temperature is adjusted and/or maintained by a cooling jacket, a cooling bath, a heating jacket, a thermocouple or combinations thereof.

In an embodiment, the mixture produced in step (b), (b1) or (b2) is separated in the presence of a solvent, in particular a organic or aqueous solvent, in particular methanol, ethyl acetate, hexanes, methylene chloride, water or combinations thereof, through fractional crystallization, preferably in a crystallization unit.

In an embodiment, the method according to the present invention further comprises: feeding the separated MHCO₃ into the hydrogenation reactor used in step (b), (b1) or (b2).

In an embodiment of the present invention, the separated HCOOM obtained is treated with a MOH, preferably a catalytic amount of a MOH, to produce a hydrogen gas and M₂(COO)₂, preferably a dried M₂(COO)₂.

In an embodiment, the separated HCOOM is treated with a MOH, preferably a catalytic amount of a MOH, at a temperature from 90° C. to 410° C., in particular 200° C. to 350° C., in particular 250° C. to 300° C., of in particular 100° C., in particular 200° C., in particular 300° C., in particular 400° C., preferably in an inert treatment reactor.

In an embodiment of the present invention, treating the separated HCOOM with MOH, in particular a catalytic amount of MOH, is preferably conducted at a temperature from 30 to 600° C., in particular 90 to 415° C., in particular 100 to 400° C., in particular 200 to 400° C., in particular 300 to 400° C., of in particular 30° C., in particular 50° C., in particular 80° C., in particular 90° C., in particular 100° C., in particular 200° C., in particular 300° C., in particular 310° C., in particular 320° C., in particular 330° C., in particular 340° C., in particular 350° C., in particular 360° C., in particular 370° C., in particular 380° C., in particular 390° C., in particular 400° C., in particular 415° C., in particular 420° C., in particular 430° C., in particular 440° C., in particular 450° C., in particular 500° C., in particular 550° C., in particular 600° C., to produce a hydrogen gas and a dried M₂(COO)₂, preferably in an inert treatment reactor.

In an embodiment of the present invention, the separated HCOOM is treated with a catalytic amount of MOH, in particular with 0.1 to 10 wt. % of MOH, in particular 1.0 to 5.0 wt. %, in particular 1.0 to 4.0 wt. %, in particular 2.0 to 3.0 wt. %, in particular 0.1 wt. %, in particular 1.0 wt. %, in particular 2.0 wt. %, in particular 3.0 wt. %, in particular 4.0 wt. %, in particular 5.0 wt. %, in particular 10 wt. % (each in relation to the amount of HCOOM).

In an embodiment of the present invention, the separated HCOOM is treated with a catalytic amount of MOH, in particular with 0.1 to 10 mol % of MOH, in particular 1.0 to 5.0 mol %, in particular 1.0 to 4.0 mol %, in particular 2.0 to 3.0 mol %, in particular 0.1 mol %, in particular 1.0 mol %, in particular 2.0 mol %, in particular 3.0 mol %, in particular 4.0 mol %, in particular 5.0 mol %, in particular 10 mol % (each in relation to the amount of HCOOM).

In an embodiment of the present invention, treating the separated HCOOM with a catalytic amount of MOH, in particular with 0.1 to 10 mol % of MOH, in particular 1.0 to 5.0 mol %, in particular 1.0 to 4.0 mol %, in particular 2.0 to 3.0 mol %, in particular 0.1 mol %, in particular 1.0 mol %, in particular 2.0 mol %, in particular 3.0 mol %, in particular 4.0 mol %, in particular 5.0 mol %, in particular 10 mol % (each in relation to the amount of HCOOM), is conducted at a temperature from 30 to 600° C., in particular 90 to 415° C., in particular 100 to 400° C., in particular 200 to 400° C., in particular 300 to 400° C., of in particular 30° C., in particular 50° C., in particular 80° C., in particular 90° C., in particular 100° C., in particular 200° C., in particular 300° C., in particular 310° C., in particular 320° C., in particular 330° C., in particular 340° C., in particular 350° C., in particular 360° C., in particular 370° C., in particular 380° C., in particular 390° C., in particular 400° C., in particular 415° C., in particular 420° C., in particular 430° C., in particular 440° C., in particular 450° C., in particular 500° C., in particular 550° C., in particular 600° C., to produce a hydrogen gas and a dried M₂(COO)₂.

In an embodiment, the MOH is selected from the group consisting of NaOH, KOH and NH₄OH and combinations thereof and preferably NaOH and/or KOH.

In an embodiment, the MOH is KOH or NaOH.

In an embodiment of the present invention, the M₂(COO)₂, preferably the dried M₂(COO)₂ is K₂(COO)₂, or Na₂(COO)₂.

In an embodiment, the method according to the present invention further comprises: treating the separated HCOOM with a MOH, preferably a catalytic amount of a MOH, preferably at a temperature from 100° C. to 400° C., to produce a hydrogen gas and M₂(COO)₂, preferably the dried M₂(COO)₂, preferably wherein the MOH is KOH or NaOH, and, preferably wherein the M₂(COO)₂, preferably the dried M₂(COO)₂ is K₂(COO)₂ or Na₂(COO)₂.

In an embodiment, the method according to the present invention further comprises:
treating the separated HCOONa with a catalytic amount of NaOH, in particular with 0.1 to 10 mol % of NaOH, in particular 1.0 to 5.0 mol %, in particular 1.0 to 4.0 mol %, in particular 2.0 to 3.0 mol %, in particular 0.1 mol %, in particular 1.0 mol %, in particular 2.0 mol %, in particular 3.0 mol %, in particular 4.0 mol %, in particular 5.0 mol %, in particular 10 mol % (each in relation to the amount of HCOONa), preferably at a temperature from 30 to 600° C., in particular 90 to 415° C., in particular 100 to 400° C., in particular 200 to 400° C., in particular 300 to 400° C., of in particular 30° C., in particular 50° C., in particular 80° C., in particular 90° C., in particular 100° C., in particular 200° C., in particular 300° C., in particular 310° C., in particular 320° C., in particular 330° C., in particular 340° C., in particular 350° C., in particular 360° C., in particular 370° C., in particular 380° C., in particular 390° C., in particular 400° C., in particular 415° C., in particular 420° C., in particular 430° C., in particular 440° C., in particular 450° C., in particular 500° C., in particular 550° C., in particular 600° C., to produce a hydrogen gas and a dried Na₂(COO)₂.

In an embodiment, the method according to the present invention further comprises:
treating the separated HCOOK with a catalytic amount of KOH, in particular with 0.1 to 10 mol % of KOH, in particular 1.0 to 5.0 mol %, in particular 1.0 to 4.0 mol %, in particular 2.0 to 3.0 mol %, in particular 0.1 mol %, in particular 1.0 mol %, in particular 2.0 mol %, in particular 3.0 mol %, in particular 4.0 mol %, in particular 5.0 mol %, in particular 10 mol % (each in relation to the amount of HCOOK), preferably at a temperature from 30 to 600° C., in particular 90 to 415° C., in particular 100 to 400° C., in particular 200 to 400° C., in particular 300 to 400° C., of in particular 30° C., in particular 50° C., in particular 80° C., in particular 90° C., in particular 100° C., in particular 200° C., in particular 300° C., in particular 310° C., in particular 320° C., in particular 330° C., in particular 340° C., in particular 350° C., in particular 360° C., in particular 370° C., in particular 380° C., in particular 390° C., in particular 400° C., in particular 415° C., in particular 420° C., in particular 430° C., in particular 440° C., in particular 450° C., in particular 500° C., in particular 550° C., in particular 600° C., to produce a hydrogen gas and a dried K₂(COO)₂.

In an embodiment, the method according to the present invention further comprises at least one step of:
transferring the hydrogen gas to the hydrogenation reactor used in step (b1); and
transferring the M₂(COO)₂, preferably the dried M₂(COO)₂, to the metal ion exchanger, in particular metal ion exchange bubble column, used in step (b1).

A further aspect of the invention is a system for generating a carbon sequestering agent, in particular a polyester, in particular a hydrogel, from a CO₂ gas stream comprising a CO₂, the system comprising:
a CO₂ conversion unit configured to convert the CO₂ into a (COOH)₂;
a (COOH)₂ conversion unit configured to convert the (COOH)₂ into the carbon sequestering agent.

In an embodiment, the (COOH)₂ conversion unit of the system according to the present invention is configured to:
convert the (COOH)₂ with a mono-alcohol into an ester, preferably in the presence of a first acid catalyst, and the ester into the carbon sequestering agent, preferably in the presence of a second acid catalyst.

In an embodiment, the CO₂ conversion unit of the system according to the present invention is configured to combine the CO₂ with a M₂(COO)₂ to produce a (COOH)₂ and a MHCO₃, preferably wherein the MHCO₃ is KHCO₃ or NaHCO₃, and wherein the M₂(COO)₂ is K₂(COO)₂ or Na₂(COO)₂.

In an embodiment, the (COOH)₂ conversion unit of the system according to the present invention further comprises a polymerization reactor configured to receive the (COOH)₂ or the ester and combine the (COOH)₂ or the ester with a polyol, in particular glycerine, in the presence of a second acid catalyst, in particular a second acid catalyst comprising a H₂SO₄, Sb₂O₃, SnCl₂, titanium isopropoxide or p-toluenesulfonic acid to produce the carbon sequestering agent, in particular the polyester and water or the mono-alcohol.

In an embodiment, the system according to the present invention further comprises:
a hydrogenation reactor connected to the CO₂ conversion unit and configured to receive the MHCO₃ from the CO₂ conversion unit and to combine the MHCO₃ with a hydrogen gas and a hydrogenation catalyst to produce a mixture comprising the MHCO₃ and a HCOOM, preferably wherein the HCOOM is HCOOK or HCOONa.

In an embodiment, the system according to the present invention further comprises:
a crystallization unit configured to receive the mixture from the hydrogenation reactor and to separate the mixture into a separated MHCO₃ and a separated HCOOM.

In an embodiment, the system according to the present invention further comprises:
Aninert treatment reactor configured to receive the separated HCOOM from the crystallization unit and to treat the separated HCOOM with MOH, in particular a catalytic amount of a MOH, preferably at a temperature from 100° C. to 400° C. to produce a hydrogen gas and a dried M₂(COO)₂, preferably wherein the M₂(COO)₂ is a K₂(COO)₂ or Na₂(COO)₂.

In an embodiment of the system according to the present invention, the separated HCOOM is treated with MOH, in particular a catalytic amount of a MOH, at a temperature from 100° C. to 400° C., in particular 200° C. to 350° C., in particular 250° C. to 300° C., in particular 100° C., in particular 200° C., in particular 300° C., in particular 400° C.

The present invention also relates to a polyester, in particular hydrogel, obtainable by a method for converting a CO₂ into a polyester, in particular a CO₂ gas stream comprising a CO₂ into the polyester, the method comprises:
(b) converting a CO₂ from the CO₂ gas stream into a (COOH)₂; and
(c) combining the (COOH)₂, a mono-alcohol (X-OH), preferably CH₃CH₂OH, and a first acid catalyst comprising a H₂SO₄ at a temperature ranging from about 80° C. to about 100° C. and under atmospheric pressure to produce an ester comprising a (COOX)₂ and preferably (COOEt)₂;
(d) the ester obtained in step (c) is reacted with a polyol, preferably glycerine to form a polyester, preferably the polyester is a hydrogel.

The polyester of the present invention is advantageous insofar as it captures CO₂, removes it from the environment and stores the CO₂ in its molecular structure, which polyester in turn makes degradation products available for various beneficial uses, in particular in agriculture. In particular, the CO₂ removed from the environment and stored in the polyester of the present invention can be used in various beneficial agricultural applications. The polyesters provided according to the present invention show a high water holding capacity, which favourably is stable over a long term and furthermore shows full reversibility in many water uptake and release cycles over a long period of time. In contrast to commercially available products, which do not undergo biodegradation and thus stay as undesired microplastics in the soil the present polyester, in particular hydrogel, according to the present invention undergoes biodegradation and improve the agricultural quality of the soil. Furthermore, in contrast to the commercially available hydrogels for agricultural purposes the polyesters according to the present invention are nitrogen free.

Advantageously, the hydrogel of the present invention when used in soil results in a very favourable modulation of the microbiome contained therein. It could be shown that the bacteria population decreases, which otherwise would consume the polyester or an agricultural composition of the present invention and release CO₂ back to atmosphere through their respiration process, while the soils fungi population increases, which is not breathing and thus releases no CO₂, and favourably decomposes the polyester into its decomposition products, which remain in the soil, thereby sequestering carbon. Furthermore, the polyesters of the present invention are favorable insofar, as they are made from non-toxic reactants and are itself non-toxic.

The polyesters of the present invention are favorable insofar, as they allow to specifically modify the composition of the microbiome in the soil, in particular decrease the microbiome diversity and increase the fungal to bacteria ratio. The biodegradability of the polyesters of the present invention is of advantage insofar, as it provides the soil with secondary metabolites, which are of use for various agricultural purposes, in particular provide important metabolites for microbes and plants and act, therefore, as fertilizer in the soil.

Thus, the polyesters of the present invention are favorable insofar, as they provide a high water holding capacity and a valuable source for various polyester biodegradation products and are made from CO₂ removed from the environment, or gas streams containing CO₂ in a simple, economic and non-toxic process.

The present invention therefore further relates to method of sequestering carbon dioxide, the method comprising the steps of:
(a) capturing carbon dioxide from an industrial gaseous waste stream and/or the atmosphere;
(b) converting a CO₂ from the CO₂ gas stream into a (COOH)₂; and
(c) combining the (COOH)₂, a mono-alcohol (X-OH), preferably CH₃CH₂OH, and a first acid catalyst comprising a H₂SO₄ at a temperature ranging from about 80° C. to about 100° C. and under atmospheric pressure to produce an ester comprising a (COOX)₂ and preferably (COOEt)₂;
(d) the ester obtained in step (c) is reacted with a polyol, preferably glycerine to form a polyester, preferably the polyester is a hydrogel.

In an embodiment the method of sequestering carbon dioxide, the polyester obtained in step (d) is a hydrogel. The method according to the invention may further comprise the step of:
(e) mixing the hydrogel with a soil and optionally nutrients, to obtain a soil-hydrogel mixture.

Carbon dioxide may be obtained from an industrial gaseous waste stream and/or the atmosphere. In case CO₂ is obtained from the atmosphere this may be done with Direct Air Capture (DAC) technologies in which CO₂ is captured from air. CO₂ may also be obtained from a gaseous waste stream such as a gaseous waste stream originating from a chemical process, steel mill and other processes generating gaseous streams containing CO₂.

In an embodiment the method of sequestering carbon dioxide, further comprises the step of:
(e) providing the polyester, preferably hydrogel, or the hydrogel-soil mixture to a soil surface, preferably a ground surface such as agricultural land, forest ground or a field. In doing so fungal growth in the ground is stimulated resulting in the storage of carbon orginating from the ester and hence results in the sequestestration of carbon dioxide. The inventors have found that good fungal growth and a good fungal to bacteria ratio in the soil is achieved with polyglycerol oxalate. Hence in an embodiment of the invention the hydrogel comprises polyglycerol oxalate and preferably consist of polyglycerol oxalate.

In an embodiment the method of sequestering carbon dioxide one or more species of fungi are added after step (c). In case the hydrogel or soil-hydrogel mixture is applied to ground surface with low amounts of fungi present, it is beneficial to add one or more species of fungi to the hydrogel or said mixture in order to achieve sufficient fungi growth in order to sequester carbon dioxide.

In an embodiment the hydrogel is mixted with seeds and nutrients and optionally soil and/or fungi. This allows for the sowing of seeds together with at least nutrients being present near the seed.

In an embodiment the method of sequestering carbon dioxide the hydrogel or hydrogel-soil mixture is shaped into particles. This step may coincide with mixing soil and hydrogel but could also be executed as a separate step by, for example, chopping the hydrogel or hydrogel-soil mixture. By reducing the size of the hydrogel or said mixture, it can be distributed over areas of soil more easily and efficiently. With distribution here, is meant in and/or on the soil. When distributed in the soil, this may be achieved during tilling or plowing of the soil.

In an embodiment, the polyester according to the present invention is obtainable by the method according to the present invention, wherein step (c) of the method further comprises: combining the (COOH)₂ with a mono-alcohol to produce an ester and (d) converting the ester and polyol into the polyester, in particular the hydrogel. Preferably, the polyol is glycerine. In an embodiment, the polyester according to the present invention is obtainable by the method according to the present invention, wherein step (b) of the method further comprises:
combining the (COOH)₂ with a mono-alcohol, in particular CH₃OH or CH₃CH₂OH, to produce an ester, in particular wherein the ester is a (COOMe)₂ or a (COOEt)₂, preferably in the presence of a first acid catalyst, in particular a first acid catalyst comprising a H₂SO₄, preferably at a temperature from 80° C. to 100° C. and preferably under atmospheric pressure.

In an embodiment, the polyester according to the present invention is obtainable by the method according to the present invention, wherein in step (d) the converting of the (COOH)₂ or the ester into a polyester comprises:
combining the (COOH)₂ or the ester with a polyol, in particular glycerine, to produce the polyester and optionally water or a mono-alcohol, preferably in the presence of a second acid catalyst, in particular a second acid catalyst comprising a H₂SO₄, Sb₂O₃, SnCl₂, titanium isopropoxide or p-toluenesulfonic acid.

In an embodiment, the polyester according to the present invention is obtainable by the method according to the present invention, wherein in step (d the converting of the ester into a polyester comprises:
combining the ester, in particular wherein the ester is (COOMe)₂ or (COOEt)₂, with a polyol, in particular glycerine, in the presence of a second acid catalyst, in particular a second acid catalyst comprising a H₂SO₄, Sb₂O₃, SnCl₂, titanium isopropoxide or p-toluenesulfonic acid, at a temperature from 100 to to 180° C., preferably 100 to 170° C., and a pressure from 0.02 bara to 1 bara to produce the polyester, in particular the hydrogel, and optionally water or a mono-alcohol, in particular a methanol or an ethanol.

In an embodiment, the polyester according to the present invention is obtainable by the method according to the present invention, wherein the polyol is glycerine and the polyester is composed of monomeric building units of (COOH)₂, and glycerine, preferably wherein the polyester, in particular hydrogel, has a structure according to Formula Iwith n ranging from 1to 5250, preferably 1 to 5000.

In an embodiment n is ranging from 500 to 4500, preferably 1000 to 4000, preferably 1500 to 3500 and in particular 2000 to 3000. In an embodiment of the present invention n is 1. In an embodiment of the present invention n is ranging from 250 to 750. In an embodiment of the present invention n is ranging from 750 to 1250. In an embodiment of the present invention n is ranging from 1250 to 1750. In an embodiment of the present invention n is ranging from 1750 to 2250. In an embodiment of the present invention n is ranging from 2250 to 2750. In an embodiment of the present invention n is ranging from 2750 to 3250. In an embodiment of the present invention n is ranging from 3250 to 3750. In an embodiment of the present invention n is ranging from 3750 to 4250. In an embodiment of the present invention n is ranging from 4250 to 4750. In an embodiment of the present invention n is ranging from 4750 to 5250.

In an embodiment, the polyester according to the present invention is obtainable by the method according to the present invention, wherein the polyol is glycerine and the polyester is composed of monomeric building units of (COOH)₂, and glycerine, preferably wherein the polyester, in particular hydrogel, has a structure according to Formula II, III or IV with n and m each ranging independently from 1 to 5250, preferably 1 to 5000.

In an embodiment, m is ranging from 500 to 4500, preferably 1000 to 4000, preferably 1500 to 3500 or preferably 2000 to 3000.

In an embodiment of the present invention m is 1. In an embodiment of the present invention m is ranging from 250 to 750. In an embodiment of the present invention m is ranging from 750 to 1250. In an embodiment of the present invention m is ranging from 1250 to 1750. In an embodiment of the present invention m is ranging from 1750 to 2250. In an embodiment of the present invention m is ranging from 2250 to 2750. In an embodiment of the present invention m is ranging from 2750 to 3250. In an embodiment of the present invention m is ranging from 3250 to 3750. In an embodiment of the present invention m is ranging from 3750 to 4250. In an embodiment of the present invention m is ranging from 4250 to 4750. In an embodiment of the present invention m is ranging from 4750 to 5250.

In an embodiment, n and m are each ranging independently from 500 to 4500, preferably 1000 to 4000, preferably 1500 to 3500 or preferably 2000 to 3000.

In an embodiment of the present invention n and m are 1. In an embodiment of the present invention n and m are each ranging independently from 250 to 750. In an embodiment of the present invention n and m are each ranging independently from 750 to 1250. In an embodiment of the present invention n and m are each ranging independently from 1250 to 1750. In an embodiment of the present invention n and m are each ranging independently from 1750 to 2250. In an embodiment of the present invention n and m are each ranging independently from 2250 to 2750. In an embodiment of the present invention n and m are each ranging independently from 2750 to 3250. In an embodiment of the present invention n and m are each ranging independently from 3250 to 3750. In an embodiment of the present invention n and m are each ranging independently from 3750 to 4250. In an embodiment of the present invention n and m are each ranging independently from 4250 to 4750. In an embodiment of the present invention n and m are each ranging independently from 4750 to 5250.

In an embodiment, the polyester according to the present invention is obtainable by the method according to the present invention, wherein the polyol is glycerine and the polyester is composed of monomeric building units of (COOH)₂, and glycerine, preferably wherein the polyester, in particular hydrogel, has a structure according to Formula I, II, III or IV with n and/or m each ranging independently from 1 to 5250, preferably 1 to 5000.

In an embodiment of the present invention, the polyester, in particular hydrogel, of the present invention is obtained by combining a (COOH)₂ ester, in particular (COOMe)₂ or (COOEt)₂, with glycerine in the presence of H₂SO₄.

In an embodiment of the present invention, the polyester, in particular hydrogel, of the present invention is obtained by combining a (COOH)₂ ester, in particular (COOMe)₂ or (COOEt)₂, with glycerine in the presence of SnCl₂.

In an embodiment of the present invention, the polyester, in particular hydrogel, of the present invention is obtained by combining a (COOH)₂ ester, in particular (COOMe)₂ or (COOEt)₂, with glycerine in the presence of Sb₂O₃.

In an embodiment of the present invention, the polyester, in particular hydrogel, of the present invention is obtained by combining a (COOH)₂ ester, in particular (COOMe)₂ or (COOEt)₂, with glycerine in the presence of titanium isopropoxide.

In an embodiment of the present invention, the polyester, in particular hydrogel, of the present invention is obtained by combining a (COOH)₂ ester, in particular (COOMe)₂ or (COOEt)₂, with glycerine in the presence of p-toluenesulfonic acid.

In an embodiment of the present invention, the polyester, in particular hydrogel, of the present invention is obtained by a method comprising steps (c) and (d), wherein step (c) requires combining the (COOH)₂ with a mono-alcohol to produce an (COOH)₂ ester, in particular (COOMe)₂ or (COOEt)₂ and further step (d) combining the (COOH)₂ ester, in particular (COOMe)₂ or (COOEt)₂, with glycerine in the presence of the second acid catalyst H₂SO₄, preferably at 100 to 160° C., more preferably 130 to 160° C.

In an embodiment of the present invention, the polyester, in particular hydrogel, of the present invention is obtained by a method comprising steps (c) and (d), wherein step (c) comprises combining the (COOH)₂ with a mono-alcohol to produce an (COOH)₂ ester, in particular (COOMe)₂ or (COOEt)₂ and further step (d) combining the (COOH)₂ ester, in particular (COOMe)₂ or (COOEt)₂, with glycerine in the presence of the second acid catalyst SnCl₂, preferably at 100 to 160° C., more preferably 130 to 160° C.

In an embodiment of the present invention, the polyester, in particular hydrogel, of the present invention is obtained by a method comprising steps (c) and (d), wherein step (c) comprises combining the (COOH)₂ with a mono-alcohol to produce an (COOH)₂ ester, in particular (COOMe)₂ or (COOEt)₂ and further step (d) combining the (COOH)₂ ester, in particular (COOMe)₂ or (COOEt)₂, with glycerine in the presence of the second acid catalyst titanium isopropoxide preferably at 100 to 180° C., more preferably 150 to 180° C.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst p-toluenesulfonic acid. preferably at 100 to 180° C., more preferably 120 to 180° C., more preferably 150 to 180° C.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst Sb₂O₃, preferably at 100 to 180° C.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst H₂SO₄, preferably 0.23 to 1.3 mol % of the second acid catalyst H₂SO₄, preferably at 100 to 160° C., more preferably 130 to 160° C.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst SnCl₂, preferably 0.04 to 0.11 mol % of the second acid catalyst SnCl₂, preferably at 100 to 160° C., more preferably 130 to 160° C.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst titanium isopropoxide preferably 0.01 to 0.48 mol % of the second acid catalyst titanium isopropoxide, preferably at 100 to 180° C., more preferably 150 to 180° C.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst p-toluenesulfonic acid preferably 0.23 to 3.83 mol % of the second acid catalyst p-toluenesulfonic acid, preferably at 100 to 180° C., more preferably 120 to 180° C., more preferably 150 to 180° C.

In an embodiment of the present invention, the polyester, in particular hydrogel, of the present invention is obtained by conducting steps (c) and (d), wherein step (c) requires combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst Sb₂O₃, preferably 0.007 to 0.77 mol % of the second acid catalyst Sb₂O₃, preferably at 100 to 180° C.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst H₂SO₄, preferably at 100 to 160° C., more preferably 130 to 160° C., wherein the polyester, in particular hydrogel, of the present invention has a water holding capacity (WHC) of 130 to 500 wt. %, in particular 130 to 260 wt. % (each in relation to the dry weight of the polyester), preferably after 30 days, preferably after 67 days, preferably 7 days, preferably determined according to method 1 or 2 of example 1.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst SnCl₂, preferably at 100 to 160° C., more preferably 130 to 160° C., wherein the polyester, in particular hydrogel, of the present invention has a water holding capacity (WHC) of 130 to 255 wt. % (in relation to the dry weight of the polyester), preferably after 30 days, preferably 67 days, preferably after 7 days, preferably determined according to method 1 or 2 of example 1.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst titanium isopropoxide preferably at 100 to 180° C., more preferably 150 to 180° C., wherein the polyester, in particular hydrogel, of the present invention has a water holding capacity (WHC) of 78 to 231 wt. %, in particular 78 to 215 wt. % (each in relation to the dry weight of the polyester), preferably after 30 days, preferably 67 days, preferably 7 days, preferably determined according to method 1 or 2 of example 1.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst p-toluenesulfonic acid, preferably at 100 to 180° C., more preferably 120 to 180° C., more preferably 150 to 180° C., wherein the polyester, in particular hydrogel, of the present invention has a water holding capacity (WHC) of 28 to 234 wt. %, in particular 28 to 202 wt. % (each in relation to the dry weight of the polyester), preferably after 30 days, preferably after 67 days, preferably 7 days, preferably determined according to method 1 or 2 of example 1.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst Sb₂O₃, preferably at 100 to 180° C., wherein the polyester, in particular hydrogel, of the present invention has a water holding capacity (WHC) of 130 wt. % (in relation to the dry weight of the polyester), preferably after 30 days, preferably after 67 days, preferably after 7 days, preferably determined according to method 1 or 2 of example 1.

In an embodiment, the polyester according to the present invention is obtainable by the method according to the present invention, wherein step (b) of the method comprises:
converting the CO₂ in a metal ion exchanger, in particular in a metal ion exchange bubble column, comprising a M₂(COO)₂ into the (COOH)₂ and a MHCO₃.

The present invention also relates to a polyester, in particular a hydrogel, according to Formula I with n ranging from 1 to 5250, preferably from 1 to 5000.

In an embodiment, n of the polyester, in particular the hydrogel, according to the present invention according to Formula I ranges from 1 to 5250, preferably 1 to 5000 and/or the weight average molecular weight of the polyester, in particular the hydrogel, according to the present invention is 400 to 50000 g/mol, in particular 1000 to 50000 g/mol, in particular 1000 to 30000 g/mol, in particular 10000 to 20000 g/mol, in particular 1000 g/mol, in particular 5000 g/mol, in particular 10000 g/mol, in particular 11789 g/mol, in particular 14420 g/mol, in particular 15000 g/mol, in particular 20000 g/mol, in particular 30000 g/mol, in particular 50000 g/mol measured by GPC and/or ESI-TOF-MS.

In an embodiment, the present invention also relates to a polyester, in particular a hydrogel, according to Formula II, III, or IVwith n and m each ranging independently from 1 to 5250, preferably 1 to 5000 and a weight average molecular weight of 400 to 50000 g/mol, in particular 1000 to 50000 g/mol, in particular 1000 to 30000 g/mol, in particular 10000 to 20000 g/mol, in particular 1000 g/mol, in particular 5000 g/mol, in particular 10000 g/mol, in particular 11789 g/mol, in particular 14420 g/mol, in particular 15000 g/mol, in particular 20000 g/mol, in particular 30000 g/mol, in particular 50000 g/mol, measured by GPC and/or ESI-TOF-MS.

In an embodiment, the present invention also relates to a polyester, in particular a hydrogel, according to Formula I, II, III, or IV with n and/or m each ranging independently from 1 to 5250, preferably 1 to 5000 and a weight average molecular weight of 400 to 50000 g/mol, in particular 1000 to 50000 g/mol, in particular 1000 to 30000 g/mol, in particular 10000 to 20000 g/mol, in particular 1000 g/mol, in particular 5000 g/mol, in particular 10000 g/mol, in particular 11789 g/mol, in particular 14420 g/mol, in particular 15000 g/mol, in particular 20000 g/mol, in particular 30000 g/mol, in particular 50000 g/mol, measured by GPC and/or ESI-TOF-MS. In an embodiment, the polyester according to the present invention has a water holding capacity (WHC) of 1 to 500 wt. %, preferably 100 to 500 wt. %, preferably 200 to 480 wt. %, preferably 200 to 300 wt. %, preferably 78 to 260 wt. %, preferably 97 to 159 wt. %, preferably 95 to 191 wt. %, preferably 89 to 215 wt. %, preferably 78 to 91 wt. %, preferably 120 to 136 wt. %, preferably 28 to 202 wt. %, preferably 36 to 78 wt. %, preferably 28 wt. %, preferably 36 wt. %, preferably 57 wt. %, preferably 84 wt. %, preferably 86 wt. %, preferably 115 wt. %, preferably 128 wt. %, preferably 143 wt. %, preferably 152 wt. %, preferably 185 wt. %, preferably 255 wt. %, preferably 260 wt. % (each in relation to the dry weight of the polyester), preferably wherein the WHC is stable for at least 38 weeks, preferably determined according to method 1 or 2 of example 1.

In an embodiment, the water holding capacity (WHC) of the polyester according to the present invention is reversible, preferably for at least 38 weeks at room temperature.

In an embodiment, the polyester according to the present invention has a cumulative biodegradation rate of at least 0 to 400, preferably 10 to 390, preferably 50 to 380, preferably 100 to 300 μmol CO₂/g dry substance of the polyester.

In an embodiment, the biodegradation, in particular the cumulative biodegradation rate, of the polyester according to the present invention is determined according to the method specified in example 3.

In an embodiment, the polyester, in particular hydrogel, according to the present invention has a hydrostability of 30 to to 90 wt. %, preferably 40 to 80 wt. %, preferably 50 to 70 wt. %, preferably 50 to 60 wt. %, preferably 37 wt. %, preferably 46 wt. %, preferably 51 wt. %, preferably 55 wt. %, preferably 66 wt. %, preferably 70 wt. %, preferably 73 wt. %, preferably 79 wt. %, preferably 89 wt. % (each in relation to the initial weight of the polyester), preferably determined according to example 4.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst titanium isopropoxide preferably 0.1 to 1 wt.-% of the second acid catalyst titanium isopropoxide, preferably at 150 to 180° C., wherein the polyester, in particular hydrogel, of the present invention has a hydrostability of 30 to 90 wt. %, in particular 37 to 89 wt. % (each in relation to the initial weight of the polyester), preferably determined according to example 4.

In an embodiment of the present invention, the polyester, in particular hydrogel, of the present invention is obtained by conducting steps (c) and (d), wherein step (c) comprises combining the (COOH)₂ with a mono-alcohol to produce an (COOH)₂ ester, in particular (COOMe)₂ or (COOEt)₂ and further comprises step (d) combining the (COOH)₂ ester, in particular (COOMe)₂ or (COOEt)₂, with glycerine in the presence of the second acid catalyst p-toluenesulfonic acid, preferably 0.3 wt.-% of the second acid catalyst p-toluenesulfonic acid, preferably at 150 to 180° C., wherein the polyester, in particular hydrogel, of the present invention has a hydrostability of 50 to 60 wt. %, in particular 51 to 55 wt. % (each in relation to the initial weight of the polyester), preferably determined according to example 4.

In an embodiment of the present invention, the hydrostability is calculated using the following equation as described in example 4:

$$Δ m = \frac{initial polyester mass ( g ) - dry polyester mass ( g )}{initial polyester mass ( g )} ,$$

wherein Δm is expressed in wt. %.

A further aspect of the present invention relates to an agricultural composition comprising a polyester, in particular hydrogel, according to the present invention, optionally in conjunction with at least one carrier and/or at least one agriculturally active additive, selected from the group consisting of an absorbent swelling clay, in particular bentonite, a pesticide, in particular an antifungal agent, and a fertilizing agent.

In an embodiment of the present invention, a carrier can be a soil or a component of a soil.

An agricultural composition comprising a polyester, in particular hydrogel, according to the present invention, can be a fertilizer.

In an embodiment of the present invention, the agricultural composition may be in dry or semi-liquid form, preferably in dry form, in particular in form of pellets, particles, tablets, powder and granules.

In an embodiment the agricultural composition of the present invention comprises 1 to 99 wt. %, preferably 2 to 95 wt. %, preferably 2 to 50 wt. %, preferably 2 to 40 wt. %, preferably 3 to 30 wt. %, preferably 4 to 25 wt. %, preferably 5 to 20 wt. % of the polyester according to the present invention and 1 to 99 wt. %, preferably 5 to 98 wt. %, preferably 50 to 98 wt. %, preferably 60 to 98 wt. %, preferably 70 to 97 wt. %, preferably 75 to 96 wt. %, preferably 80 to 95 wt. % (each based on overall weight) of the at least one agriculturally active additive and/or the at least one carrier.

In an embodiment the agricultural composition of the present invention comprises 1 to 99 wt. %, preferably 2 to 95 wt. %, preferably 2 to 50 wt. %, preferably 2 to 40 wt. %, preferably 3 to 30 wt. %, preferably 4 to 25 wt. %, preferably 5 to 20 wt. % of the polyester according to the present invention and 1 to 99 wt. %, preferably 5 to 98 wt. %, preferably 50 to 98 wt. %, preferably 60 to 98 wt. %, preferably 70 to 97 wt. %, preferably 75 to 96 wt. %, preferably 80 to 95 wt. % (each based on overall weight) of the at least one agriculturally active additive.

In an embodiment the agricultural composition of the present invention comprises 1 to 99 wt. %, preferably 2 to 95 wt. %, preferably 2 to 50 wt. %, preferably 2 to 40 wt. %, preferably 3 to 30 wt. %, preferably 4 to 25 wt. %, preferably 5 to 20 wt. % of the polyester according to the present invention and 1 to 99 wt. %, preferably 5 to 98 wt. %, preferably 50 to 98 wt. %, preferably 60 to 98 wt. %, preferably 70 to 97 wt. %, preferably 75 to 96 wt. %, preferably 80 to 95 wt. % (each based on overall weight) of the at least one carrier.

In an embodiment, the agricultural composition of the present invention comprises bentonite, in particular 1 to 99 wt. %, preferably 2 to 95 wt. %, preferably 1 to 55 wt. %, in particular 1 to 50 wt. %, in particular 10 to 40 wt. %, in particular 20 to 30 wt. %, preferably 50 to 99 wt. %, preferably 60 to 95 wt. %, preferably 75 to 95 wt. %, in particular 1 wt. %, in particular 10 wt. %, in particular 20 wt. %, in particular 30 wt. %, in particular 40 wt. %, in particular 50 wt. %, in particular 55 wt. % bentonite (each based on overall weight).

A further aspect of the present invention relates to a method to prepare an agricultural composition according to the present invention comprising mixing a polyester, in particular hydrogel, according to the present invention with at least one agriculturally active additive, at least one carrier or both.

A further aspect of the present invention relates to a method to fertilize a soil comprising adding a polyester, in particular hydrogel, or an agricultural composition according to the present invention to a soil or a sample of a soil.

A further aspect of the present invention relates to a method to increase at least one agricultural quality of a soil comprising adding a polyester, in particular hydrogel, or an agricultural composition according to the present invention to a soil, preferably in situ, or to an isolated sample of a soil. Thus, the present invention provides a method, which requires adding a polyester, in particular a hydrogel of the present invention, or an agricultural composition of the present invention to a soil thereby resulting in a soil, which exhibits at least one increased agricultural quality in comparison to a soil, to which no polyester, in particular no hydrogel, or no agricultural composition has been added. An increased agricultural quality is meant to refer to one characteristic feature of a soil, that means a quality of a soil, whose agricultural quality has been increased in comparison to a soil, to which no polyester, in particular no hydrogel of the present invention, or no agricultural composition of the present invention has been added.

In the context of the present invention, a quality of a soil is a measure of the condition of a soil in relation to the requirements of one or more biotic species and/or to any human needs or purpose, in particular it refers to functions of the soil. Soil quality is the capacity of a soil to function, within natural or managed ecosystems, to sustain microbial, plant and/or animal productivity, maintain or enhance water and air quality and/or availability, and support human health and habitation.

In particular, the quality of a soil reflects to which extent a soil performs the functions of maintaining biodiversity and productivity, partitioning water and solute flow, filtering and buffering, nutrient cycling, and providing support for microbes, plants and other structures.

In an embodiment, one quality of the soil increased by the method according to the present invention is an increased or stored carbon content, in particular C (carbon) content, in the soil. In the context of the present invention, the term “stored carbon content” refers to the ability of the polyester, in particular hydrogel, in particular agricultural composition of the present invention, to maintain a quantitively stable carbon content in an agriculturally used soil and thereby provide the specific advantage of maintaining a quantitively stable carbon content in the soil, which is of beneficial agricultural value.

In an embodiment, one quality of the soil increased by the method according to the present invention is the water holding capacity of the soil.

In an embodiment, the increased water holding capacity of the soil is stable, in particular for at least two months. Preferably, the water holding capacity (also termed WHC) determined according to method 1 of example 1 is calculated using the following equation:

$$WHC = \frac{Wet sample weight - oven dry sample weight}{Oven dry sample weight} ,$$

wherein WHC is expressed in wt. %.

In an embodiment, the water holding capacity of a polyester according to the present invention is determined by mixing a polyester according to the present invention with water, in particular with 1 to 1000 wt. % water, in particular 100 to 500 wt %, in particular 200 to 400 wt. %, in particular 100 wt. %, in particular 200 wt. %, in particular 300 wt. %, in particular 400 wt. %, in particular 500 wt. % (each in relation to the weight of the polyester according to the present invention) water, preferably according to method 1 of example 1.

In an embodiment, the water holding capacity is determined at a temperature from 20 to 40° C., in particular 25 to 35° C., of in particular 20° C., in particular at 25° C., in particular 30° C., in particular 35° C., in particular 40° C.

In an embodiment, the water holding capacity is determined at a moisture content in the air from 1 to 100%, in particular 10 to 80%, in particular 20 to 30%, preferably at 20° C. and 0.02 bar.

In an embodiment, the water holding capacity is determined over a timeframe of 10 to 4500 hours, in particular 800 to 2700 hours, in particular 800 hours, in particular 2700 hours, in particular 4500 hours.

In an embodiment, the water holding capacity is determined at a temperature from 20 to 40° C., in particular 25 to 35° C., of in particular 20° C., in particular at 25° C., in particular 30° C., in particular 35° C., in particular 40° C. and a moisture content in the air of 1 to 100%, in particular 10 to 80%, in particular 20 to 30%, preferably at 20° C. and 0.02 bar.

In an embodiment, the water holding capacity is determined at a temperature from 20 to 40° C., in particular 25 to 35° C., of in particular 20° C., in particular at 25° C., in particular 30° C., in particular 35° C., in particular 40° C., a moisture content in the air of 1 to 100%, in particular 10 to 80%, in particular 20 to 30%, preferably at 20° C. and 0.02 bar and over a timeframe of 10 to 4500 hours, in particular 800 to 2700 hours, in particular 800 hours, in particular 2700 hours, in particular 4500 hours.

In an embodiment, the water holding capacity is determined by mixing a polyester according to the present invention and water, in particular 1 to 1000 wt. % water (in relation to the weight of the polyester according to the present invention), at a temperature from 20 to 40° C., in particular 25 to 35° C., of in particular 20° C., in particular at 25° C., in particular 30° C., in particular 35° C., in particular 40° C., a moisture content in the air of 1 to 100%, in particular 10 to 80%, in particular 20 to 30%, preferably at 20° C. and 0.02 bar and over a timeframe of 10 to 4500 hours, in particular 800 to 2700 hours, in particular 800 hours, in particular 2700 hours, in particular 4500 hours.

In a furthermore preferred embodiment of the present invention, the increased water holding capacity of the soil is reversible, in particular reversible for at least 38 weeks.

In an embodiment, the reversibility of the water holding capacity is determined at two different temperatures, in particular at two different temperatures in the range from 20 to 40° C., in particular 25 to 35° C., in particular at 25° C. and 35° C.

In an embodiment, the reversibility of the water holding capacity is determined over a timeframe of 10 to 6500 hours, in particular 800 to 6000 hours, in particular 800 hours, in particular 2700 hours, in particular 6000 hours, in particular 6500 hours.

In an embodiment, the reversibility of the water holding capacity lasts at least over 1 to 1000 uptake and release cycles, in particular 10 to 500 uptake and release cycles, at least 20 to 100 uptake and release cycles, in particular 30 to 80 uptake and release cycles, in particular 40 to 50 uptake and release cycles, in particular 10 uptake and release cycles, in particular 20 uptake and release cycles, in particular 30 uptake and release cycles, in particular 40 uptake and release cycles, in particular 50 uptake and release cycles, in particular 80 uptake and release cycles, in particular 100 uptake and release cycles, in particular 500 uptake and release cycles, in particular 1000 uptake and release cycles.

In an embodiment, the reversibility of the water holding capacity is determined at two different temperatures, in particular at two different temperatures in the range from 20 to 40° C., in particular 25 to 35° C., in particular at 25° C. and 35° C. and over a timeframe of 10 to 6500 hours, in particular 800 to 6000 hours, in particular 800 hours, in particular 2700 hours, in particular 6000 hours, in particular 6500 hours.

In an embodiment, the reversibility of the water holding capacity is determined at two different temperatures, in particular at two different temperatures in the range from 20 to 40° C., in particular 25 to 35° C., in particular at 25° C. and 35° C., over a timeframe of 10 to 6500 hours, in particular 800 to 6000 hours, in particular 800 hours, in particular 2700 hours, in particular 6000 hours, in particular 6500 hours and lasts at least over 1 to 1000 uptake and release cycles, in particular 10 to 500 uptake and release cycles, at least 20 to 100 uptake and release cycles, in particular 30 to 80 uptake and release cycles, in particular 40 to 50 uptake and release cycles, in particular 10 uptake and release cycles, in particular 20 uptake and release cycles, in particular 30 uptake and release cycles, in particular 40 uptake and release cycles, in particular 50 uptake and release cycles, in particular 80 uptake and release cycles, in particular 100 uptake and release cycles, in particular 500 uptake and release cycles, in particular 1000 uptake and release cycles.

In particular, the water holding capacity and/or reversibility is given under the circumstances as referred to in method 2 of example 1.

In an embodiment, one quality of the soil increased by the method according to the present invention is a decreased microbiome diversity of the microbiome of the soil, in particular a quantitative reduction of Acidobacteria and Chloroflexi, optionally Bacteroidetes, optionally a disappearance of Thaumarchaeota, a quantitative increase of Proteobacteria, in particular Alphaprotobacteria, optionally Actinobacteria, and optionally an appearance of Ascomycota or combinations thereof.

In an embodiment, the decreased microbiome diversity in the soil is alpha diversity, beta diversity or both. Preferably the microbiome diversity in the soil is determined by mixing soil with a polyester according to the present invention, Bentonite, an agricultural composition according to the present invention or a commercially available hydrogel and analysing a DNA sample. Preferably the DNA sample is analysed after an incubation time of the soil mixed with the polyester according to the present invention, Bentonite an agricultural composition according to the present invention or the commercially available hydrogel ranging from 1 to 2500 hours, in particular 10 to 2200 hours, in particular 50 to 2000 hours, of in particular 1 hour, in particular 10 hours, in particular 50 hours, in particular 300 hours, in particular 500 hours, in particular 1000 hours, in particular 2000 hours, in particular 2184 hours, in particular 2200 hours, in particular 2500 hours. Preferably, the alpha diversity is determined using the Shannon-Weaver index. Preferably, the beta diversity is determined using the Bray-Curtis dissimilarity method. In an embodiment, the microbiome diversity in the soil, in particular alpha and/or beta diversity, is determined according to the methods specified in example 2.

In an embodiment, one quality of the soil increased by the method according to the present invention is an increased fungal to bacteria ratio in the soil. Preferably the fungal to bacteria ratio in the soil is determined by mixing soil with a polyester according to the present invention, Bentonite, an agricultural composition according to the present invention or a commercially available hydrogel and analysing the DNA sample. Preferably the DNA sample is analysed after an incubation time of the soil mixed with the polyester according to the present invention, Bentonite, an agricultural composition according to the present invention or the commercially available hydrogel ranging from 1 to 2500 hours, in particular 10 to 2200 hours, in particular 50 to 2000 hours, of in particular 1 hour, in particular 10 hours, in particular 50 hours, in particular 300 hours, in particular 500 hours, in particular 1000 hours, in particular 2000 hours, in particular 2184 hours, in particular 2200 hours, in particular 2500 hours.

In an embodiment, the increased fungal to bacteria ratio in the soil is determined according to the method specified in example 2.

In an embodiment, one quality of the soil increased by the method according to the present invention is an increased content of secondary metabolites, preferably formate, oxalate and/or glycerine.

In an embodiment of the present invention, the increased content of secondary metabolites is due to the presence of the added polyester of the present invention which is biodegradable in the soil.

In an embodiment of the present invention, the biodegradability and the increased content of secondary metabolites formed by the biodegradation of the polyester of the present invention in the soil is determined at a constant temperature ranging from 20 to 40° C., in particular 25 to 35° C., of in particular 20° C., in particular at 25° C., in particular 30° C., in particular 35° C., in particular 40° C.

In an embodiment of the present invention, the biodegradability and the increased content of secondary metabolites formed by the biodegradation of the polyester of the present invention in the soil is determined over a timeframe ranging from 1 to 2184 hours, in particular 100 to 2000 hours, in particular 500 to 1000 hours, of in particular 1 hour, in particular 100 hours, in particular 500 hours, in particular 1000 hours, in particular 2000 hours, in particular 2184hours.

In an embodiment of the present invention, the biodegradability and the increased content of secondary metabolites formed by the biodegradation of the polyester of the present invention in the soil is determined mixing a soil with a polyester of the present invention, in particular with 0.1 to 100 wt. %, in particular 0.1 to 10 wt. %, in particular 0.1 to 5 wt. %, in particular 0.1 to 1 wt. %, in particular 0.1 wt. %, in particular 1 wt. %, in particular 5 wt. %, in particular 10 wt. %, in particular 100 wt. % of the polyester of the present invention (each in relation to the weight of the soil).

In an embodiment of the present invention, the biodegradability and the increased content of secondary metabolites formed by the biodegradation of the polyester of the present invention in the soil is determined at a constant water holding capacity of the polyester of the present invention, in particular a water holding capacity ranging from 1 to 1000%, in particular to 10% to 500%, in particular 40 to 400%, in particular 60 to 300%, in particular 100 to 200%, of in particular 1%, in particular 10%, in particular 40%, in particular 60%, in particular 65%, in particular 100%, in particular 200%, in particular 300%, in particular 400%, in particular 500%, in particular 1000%.

In an embodiment of the present invention, the biodegradability and the increased content of secondary metabolites formed by the biodegradation of the polyester of the present invention in the soil is determined at a constant temperature ranging from 20 to 40° C., in particular 25 to 35° C., of in particular 20° C., in particular at 25° C., in particular 30° C., in particular 35° C., in particular 40° C., a constant water holding capacity of the polyester according to the present invention ranging from 1 to 1000%, in particular to 10% to 500%, in particular 40 to 400%, in particular 60 to 300%, in particular 100 to 200%, of in particular 1%, in particular 10%, in particular 40%, in particular 60%, in particular 65%, in particular 100%, in particular 200%, in particular 300%, in particular 400%, in particular 500%, in particular 1000% and over a timeframe ranging from 1 to 2184 hours, in particular 100 to 2000 hours, in particular 500 to 1000 hours, of in particular 1 hour, in particular 100 hours, in particular 500 hours, in particular 1000 hours, in particular 2000 hours, in particular 2184 hours.

In an embodiment of the present invention, the biodegradability and the increased content of secondary metabolites formed by the biodegradation of the polyester of the present invention in the soil is determined according to the method specified in example 3.

A further aspect of the present invention is a soil, which soil comprises a polyester, in particular hydrogel, or an agricultural composition according to the present invention, preferably obtainable by a method according to the present invention, which soil preferably has at least one increased agricultural quality.

The present invention thus provides a soil comprising a polyester, in particular hydrogel, or an agricultural composition according to the present invention. In an embodiment, the soil is contained in a container or other form of packaging.

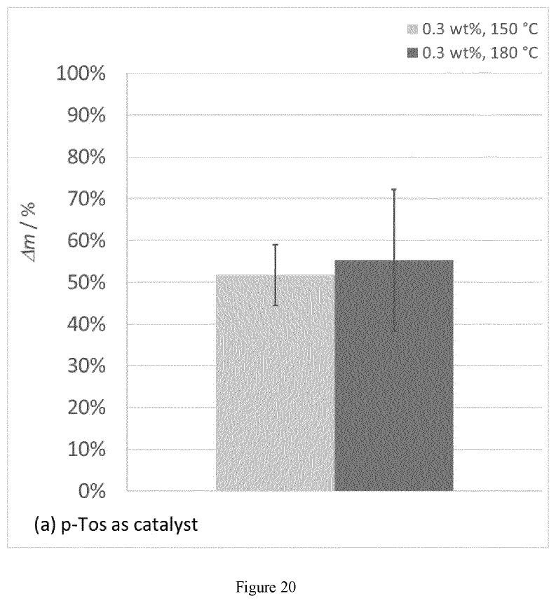

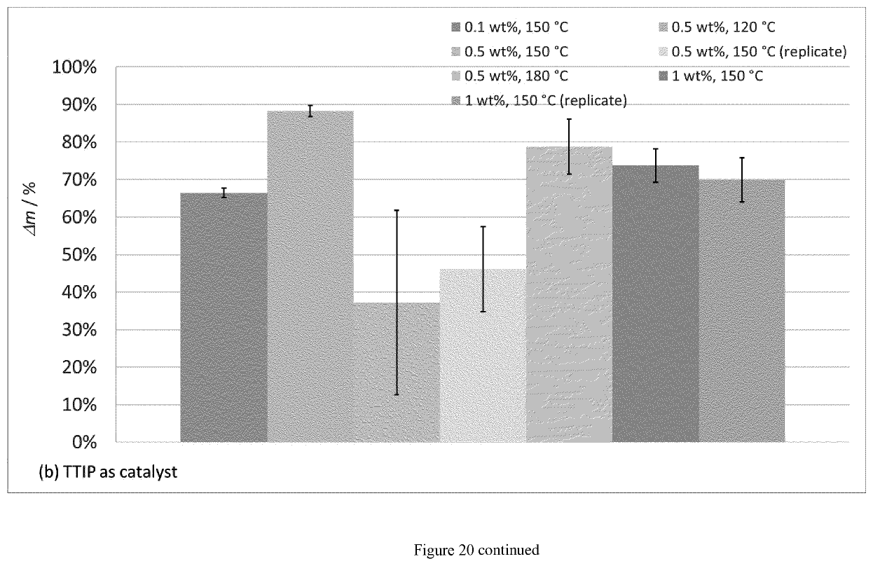

The present invention preferably relates to a method for converting a CO₂ into a polyester, as described earlier in the present disclosure, preferably from a CO₂ gas stream comprising or consisting of CO₂ into a polyester. The method comprises the steps ofconverting the CO₂ into a (COOH)₂, combining the (COOH)₂, a mono-alcohol (X-OH), preferably CH₃CH₂OH, and a first acid catalyst comprising a H₂SO₄ at a temperature ranging from about 80° C. to about 100° C. and under atmospheric pressure to produce an ester comprising a (COOEt)₂ and the ester obtained in the previous step is reacted with a polyol, preferably glycerine to form a polyester, preferably the polyester is a hydrogel.

wherein in an embodiment the (COOH)₂ is produced from M₂(COO)₂ by ion exchange in a metal ion exchanger, in particular a metal ion exchange bubble column, and wherein in aloading phase of said process the M₂(COO)₂ to be used in the metal ion exchanger, in particular metal ion exchange bubble column, for the ion exchange to produce (COOH)₂ is prepared from a CO₂, in particular a CO₂ gas stream, and wherein in a continuous process in one or more cycles of a second series of process steps the polyester of the present invention is prepared. Thus, this particularly preferred embodiment comprises a first series of process steps to load the metal ion exchanger, in particular metal ion exchange bubble column, with M₂(COO)₂ and requires one or more cycles of a second series of process steps to obtain the polyester. Thus, according to this preferred embodiment and as shown in FIG. 20, in a series of loading process steps of the method for producing a polyester, in particular a hydrogel, from a CO₂. in particular a CO₂ gas stream, the method includes a first step (1.1 in FIG. 20) of passing a CO₂ gas stream including a CO₂ through a water bath to produce a carbonated water. The carbonated water is passed in a second step (1.2 in FIG. 20) through a metal ion exchanger, in particular a metal ion exchange bubble column, which comprises a MOH to produce a MHCO₃ and an off gas. In a third step (1.3 in FIG. 20) the MHCO₃ produced from the metal ion exchanger, in particular the metal ion exchange bubble column, is combined with a H₂ in a hydrogenation reactor comprising a hydrogenation catalyst, preferably at a temperature ranging from 15° C. to 150° C. and/or a pressure ranging from 0.1 bara to 100 bara, to produce a MHCO₃ and HCOOM mixture. A fourth step (1.4 in FIG. 20) includes separating the MHCO₃ and HCOOM mixture, preferably through fractional crystallization, preferably in a crystallization unit, into a separated HCOOM and a separated MHCO₃. The separated MHCO₃ is fed to the hydrogenation reactor used in the third step. A fifth step (1.5 in FIG. 20) includes treating the HCOOM, preferably by way of an inert thermal treatment in a dryer or inert treatment reactor, with MOH, preferably a catalytic amount of MOH, preferably at a temperature ranging from 100° C. to 400° C., to produce a hydrogen gas and a dried M₂(COO)₂. The produced hydrogen gas is transferred to the hydrogenation reactor used in the third step (1.3 in FIG. 20). The dried M₂(COO)₂ is transferred to the metal ion exchanger, in particular the metal ion exchange bubble column, used in the second step (1.2 in FIG. 20).

This dried M₂(COO)₂ obtained in the loading phase is used then in a second series of process steps of the present method for producing a polyester, which second series of process steps is preferably conducted in one or more cycles, preferably in a continuous process. The dried M₂(COO)₂ obtained in the loading phase is used in the metal ion exchanger to produce (COOH)₂. Thus, in a first step (2.1 in FIG. 20) of the second series of process steps it is preferred to pass a CO₂, in particular CO₂ gas stream, including a CO₂ through a water bath to produce a carbonated water. A second step (2.2 in FIG. 20) includes passing the carbonated water through the metal ion exchanger, in particular metal ion exchange bubble column, including the M₂(COO)₂ from the fifth step of the loading process steps (1.5 in FIG. 20) to produce a (COOH)₂ and a MHCO₃. The produced MHCO₃ of the second step of the second series of process steps (2.2 in FIG. 20), is transferred to the hydrogenation reactor and the third to fifth steps of the loading process steps (1.3 to 1.5 in FIG. 20) are carried out to produce a further dried M₂(COO)₂, which is then used in the metal ion exchanger, in particular metal ion exchange bubble column to produce a further (COOH)₂ (2.1 and 2.2 in FIG. 20). An optional third step of the second series of process steps (2.3 in FIG. 20) includes combining the (COOH)₂ with a mono-alcohol, in particular CH₃OH or CH₃CH₂OH, to produce an ester, in particular, wherein the ester is a (COOMe)₂ or a (COOEt)₂, preferably in the presence of a first acid catalyst, in particular a first acid catalyst comprising a H₂SO₄, preferably at a temperature from 80° C. to 100° C. and preferably under atmospheric pressure, preferably by passing a (COOH)₂ through an activated carbon bed to produce a (COOH)₂ absorbed carbon bed comprising an absorbed (COOH)₂ and passing a mono-alcohol, in particular a methanol or an ethanol, through the (COOH)₂ absorbed carbon bed to produce an ester, in particular a (COOCH₃)₂ or a (COOEt)₂. A fourth step (2.4 in FIG. 20) includes combining the (COOH)₂ produced in the second step (2.2 in FIG. 20) or optionally the ester produced in the third step (2.3 in FIG. 20) with a polyol, in particular glycerine, to produce a polyester and optionally water or a mono-alcohol, in particular a hydrogel and optionally water or a mono-alcohol, preferably in the presence of a second acid catalyst, in particular a second acid catalyst comprising a H₂SO₄, Sb₂O₃, SnCl₂, titanium isopropoxide or p-toluenesulfonic acid.

In an embodiment, the steps of the second series of process steps using the produced MHCO₃ of the second step of the second series of process steps (2.2 in FIG. 20) to produce dried M₂(COO)₂ correspond to the third to fifth step of the loading process steps (1.3 to 1.5 in FIG. 20) of the method according to the present invention.

In some embodiments, a method may include a step of passing a CO₂ gas stream including a CO₂ through a water bath to produce a carbonated water. A carbonated water may be passed through a metal ion exchange bubble column including a M₂(COO)₂ to produce a (COOH)₂ and a MHCO₃. A method may include a step of passing a (COOH)₂ through an activated carbon bed to produce a (COOH)₂ absorbed carbon bed comprising an absorbed (COOH)₂ and passing an alcohol comprising one or more of a methanol and an ethanol through the (COOH)₂ absorbed carbon bed to produce a one or more of a (COOCH₃)₂ and a (COOEt)₂. A method may include combining a glycerine, an acid catalyst comprising a H₂SO₄, and one or more of a (COOCH₃)₂ and a (COOEt)₂ produced from the metal ion exchange bubble column to produce a hydrogel and an ethanol. In some embodiments, a method include a step of combining the MHCO₃ produced from a metal ion exchange bubble column with a hydrogen gas in a hydrogenation reactor comprising a palladium catalyst at a temperature range of about 15 to about 150° C. and a pressure of about 0.1 bara to about 100 bara to produce a MHCO₃ and HCOOM mixture and a step of separating the MHCO₃ and HCOOM mixture through fractional crystallization in a crystallization unit into a separated MHCO₃ that may be recycled back to the hydrogenation reactor and a separated HCOOM. A method may include treating a HCOOM to an inert thermal treatment in dryer/inert treatment reactor with a catalytic amount of MOH at a temperature ranging from about 100° C. to about 400° C. to produce a hydrogen gas that may be transferred to a hydrogenation reactor and a dried M₂(COO)₂ that may be transferred to the metal ion exchange bubble column. In some embodiments, M=Na (sodium) and K (potassium). According to some embodiments, a M₂(COO)₂ may include one or more of K₂(COO)₂ and Na₂(COO)₂. A HCOOM may include one or more of HCOOK and HCOONa. A MHCO₃ may include one or more of NaHCO₃ and NaHCO₃. A MOH may include one or more of KOH and NaOH.

According to some embodiments, the present disclosure relates to a system for generating a hydrogel from a CO₂ gas stream. A system may include a metal ion exchange bubble column that may be connected to a CO₂ gas stream source through a CO₂ gas inlet, to a polymerization unit through a (COOEt)₂ transfer line, to a dryer/inert treatment reactor through a M₂(COO)₂ transfer line, and a hydrogenation reactor through a MHCO₃ transfer line. A metal ion exchange bubble column may be configured to combine a CO₂ gas stream containing a CO₂ with a M₂(COO)₂ to produce a (COOEt)₂ and a MHCO₃. A system may include a polymerization unit that may be configured to receive a (COOEt)₂ from a metal ion exchange bubble column through a (COOEt)₂ transfer line and to combining a glycerine, an acid catalyst comprising a H₂SO₄, and the (COOEt)₂ to produce a hydrogel and an ethanol.

In some embodiments, a system may include a hydrogenation reactor that may be configured to receive a MHCO₃ from a metal ion exchange bubble column through a MHCO₃ transfer line, the hydrogenation reactor connected to a crystallization unit through a MHCO₃/HCOOM mixture transfer line. A hydrogenation reactor may be configured to combine the MHCO₃ with a hydrogen gas and a palladium catalyst at a temperature range of about 15 to about 150° C. and a pressure range from about 0.1 bara to about 100 bara to produce a MHCO₃ and HCOOM mixture. A system may include a crystallization unit that may be configured to receive a MHCO₃ and HCOOM mixture through a MHCO₃/HCOOM mixture transfer line. A crystallization unit may be connected to a dryer/inert treatment reactor through a HCOOM transfer line and the crystallization unit may be configured to separate a MHCO₃ and HCOOM mixture through fractional crystallization into a separated MHCO₃ and a separated HCOOM.

In some embodiments, a system may include a dryer/inert treatment reactor that may be configured to receive a separated HCOOM from a crystallization unit and to treat a HCOOM to an inert thermal treatment with a catalytic amount of KOH at a temperature ranging from about 100° C. to about 400° C. to produce a hydrogen gas and a dried M₂(COO)₂.

The present disclosure relates, according to some embodiments, to a use of a hydrogel produced from a CO₂ gas stream to sequester carbon in soils. A use may include the use comprising the step of combining a hydrogel with a soil. A hydrogel may be formed by various steps as disclosed herein.

### Hydrogel

The embodiments disclosed earlier in the present disclosure with respect to the hydrogel may also be combined with the embodiments disclosed below with respect to the hydrogel and the use of said hydrogel.

According to some embodiments, a hydrogel includes a polymer (e.g., polyester) made from a polymerization reaction performed by combining a glycerine and a (COOEt)₂ in the presence of an acid catalyst (e.g., H₂SO₄). A disclosed polymer may have a structure according to Formula I, II, III, or IV. A hydrogel according to Formula I may include an n value ranging from about 1 to about 5,000. In some embodiments, a hydrogel according to Formula I may include a n value of about 1, or of about 500, or of about 1,000, or of about 1,500, or of about 2,000, or of about 2,500, or of about 3,000, or of about 3,500, or of about 4,000, or of about 4,500, or of about 5,000, where about includes plus or minus 250.

In an embodiment the hydrogel or a mixture comprising said hydrogel is used for sequestering carbon dioxide by promoting growth of fungi. As fungi store carbons and do not emit CO₂, the carbons originating from the hydrogel are stored. As, as a raw material, carbon dioxide is used to obtain the hydrogel, the storage of these carbons from the hydrogel results in the sequestration of carbon dioxide.

In an embodiment at least part of the fungi are present in the ground preferably said ground is agricultural land, forest ground or a field.

In an embodiment one or more fungi species are present in the hydrogel or the mixture comprising hydrogel. The inventors have found that good fungal growth and a good fungal to bacteria ratio is achieved with polyglycerol oxalate. Hence in an embodiment of the invention the hydrogel comprises polyglycerol oxalate and preferably consist of polyglycerol oxalate.

In an embodiment the the hydrogel or mixture comprising hydrogel further comprises one or more plant nutrients. Plant nutrients are for example manure or compost or compounds belonging to one or more of the group consisting of: a phospholipid, a phosphoprotein, a phosphoester, a sugar phosphate, and a phytate.

The hydrogel has a water holding capacity (WHC) of 130 to 500 wt % (water weight in relation to the dry weight of the polyester). In use the hydrogel may be partly or entirely saturated with water.

### Systems for Converting a CO₂ to a Hydrogel

According to some embodiments, as shown in FIG. 1, a disclosed system 100 for converting a CO₂ to a hydrogel may include a metal ion exchange bubble column 110 connected to a CO₂ gas stream 105 through a CO₂ gas transfer line, to a hydrogenation reactor 115 through a MHCO₃ transfer line and a EtOH transfer line, to a polymerization reactor 130 through a (COOMe)₂ transfer line, and to a dryer/inert treatment reactor 125 through a M₂(COO)₂ transfer line. In some embodiments, a hydrogenation reactor 115 may be connected to a H₂ tank 145 through a H₂ transfer line, to a crystallization unit 120 through both a MHCO₃/MOOCH transfer line and a MHCO₃ transfer line, and to a dryer/inert treatment reactor 125 through a H₂ transfer line. A crystallization unit 120 may be connected to a dryer/inert treatment reactor 125 through a MOOCH transfer line. A polymerization reactor may be connected to a 2nd reactant stream 135 through a 2nd reactant transfer line and may be connected to a hydrogel tank 140 through a hydrogel transfer line.

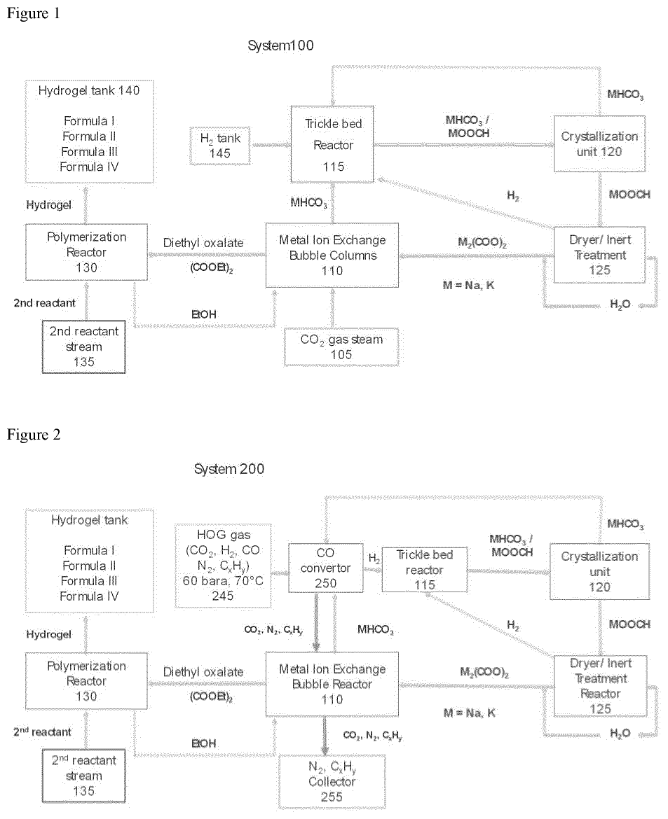

In some embodiments, as shown in FIG. 2, a system 200 includes an off Gas tank 245, a CO convertor 250, and a N₂, CₓH_{y} Collector 255. A CO converter 250 may be connected to an off gas tank 245 through a off Gas transfer line, connected to a crystallization unit 120 through a MHCO₃ transfer line, connected to a metal ion exchange bubble column 110 through both a MHCO₃ transfer line and a CO₂, N₂, CₓH_{y} transfer line, and connected to a hydrogenation reactor 115 through a hydrogenation reactor transfer line.

As shown in FIG. 1, a system may include a CO₂ gas stream 105, which may be provided by a tank or streamed from a CO₂ gas source such as a device containing a fuel combustion or processing component. A CO₂ gas stream 105 may be connected to a metal ion exchange bubble column 110 through a CO₂ gas transfer line and may configured to transfer a CO₂ gas through the CO₂ gas transfer line. Within a metal ion exchange bubble column 110, a CO₂ gas may be combined with a base including one or more of KOH and NaOH to produce one or more of a KHCO₃ and a NaHCO₃. A metal ion exchange bubble column 110 includes a bubble column including a vertically-arranged or horizontally arranged column of any shape and size. In some embodiments, a CO₂ gas transfer line may provide a CO₂ gas at a bottom, at a top, or in a position between the bottom and the top of a bubble column. For example, a metal ion exchange bubble column 110 may be configured to receive a CO₂ gas from a CO₂ gas transfer line at a position of a bubble column found in the lower half of the bubble column. An ion exchange bubble column 110 may be at a temperature ranging from about 5° C. to about 60° C. For example, an ion exchange bubble column 110 may be at a temperature of about 5° C., or about 10° C., or about 15° C., or about 20° C., or about 25° C., or about 30° C., or about 35° C., or about 40° C., or about 45° C., or about 50° C., or about 55° C., or about 60° C., where about includes plus or minus 2.5° C. In some embodiments, an ion exchange bubble column 110 may produce one or more of a MHCO₃, which may precipitate at a bottom of the ion exchange bubble column 110. A solution containing a MHCO₃ may be transferred from an ion exchange bubble column 110 to a CO convertor 250 through a MHCO₃ transfer line.

In some embodiments, as shown in FIG. 1, a system 100 may include a hydrogenation reactor 115 containing a catalyst including one or more of a palladium catalyst, a nickel catalyst, and a platinum catalyst. A catalyst includes, but is not limited to, a Pd/C, a Pd/Al₂O₃, a Ni/SiO₂, a Pd/theta Al₂O₃, a SiO₂/Al₂O₃, and combinations thereof. A method includes using a catalyst at a concentration ranging from about 0.1 g/100 mL of solvent (e.g., organic or inorganic) to about 5 g/100 mL of solvent. A method includes using a catalyst at a concentration of about 0.1 g/100 mL of solvent, or about 0.5 g/100 mL of solvent, or about 1.0 g/100 mL of solvent, or about 1.5 g/100 mL of solvent, or about 2.0 g/100 mL of solvent, or about 2.5 g/100 mL of solvent, or about 3.0 g/100 mL of solvent, or about 3.5 g/100 mL of solvent, or about 4.0 g/100 mL of solvent, or about 4.5 g/100 mL of solvent, or about 5.0 g/100 mL of solvent, where about includes plus or minus 0.25 g/100 mL of solvent. As shown in FIG. 1, a hydrogenation reactor 115 may be connected to a H₂ tank 145 through a H₂ transfer line, to a crystallization unit 120 through both a MHCO₃/MOOCH transfer line and a MHCO₃ transfer line, and to a dryer/inert treatment reactor 125 through a H₂ transfer line. In some embodiments, a hydrogenation reactor 115 may be configured to receive a MHCO₃ from a metal ion exchange bubble column and to combine the MHCO₃ with a hydrogen gas and a palladium catalyst at a temperature ranging from about of 15° C. to about 150° C. and a pressure ranging from about 0.1 bara to about 100 bara to produce a MHCO₃ and HCOOM mixture. A hydrogenation reactor 115 contains a MHCO₃ at a concentration ranging from about 0.5 M to about 5 M. A hydrogenation reactor 115 may contain a MHCO₃ at a concentration of about 0.5 M, or about 1.0 M, or about 1.5 M, or about 2.0 M, or about 2.5 M, or about 3.0 M, or about 3.5 M, or about 4.0 M, or about 4.5 M, or about 5.0 M, where about includes plus or minus 0.25 M. In some embodiments, a hydrogenation reactor 115 may contain from about 50 mL to about 1,000 mL of a MHCO₃ solution. In some embodiments, a liquid space velocity inside a hydrogenation reactor 115 includes a range from about 0.1 1/h to about 5 1/h. A hydrogenation reactor 115 may include a temperature of about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C., or about 85° C., or about 90° C., or about 95° C., or about 100° C., or about 105° C., or about 110° C., or about 115° C., or about 120° C., or about 125° C., or about 130° C., or about 135° C., or about 140° C., or about 145° C., or about 150° C., where about includes plus or minus 2.5° C. According to some embodiments, a hydrogenation reactor 115 may include a hydrogen pressure of about 0.001 bara, or about 0.005 bara, or about 0.01 bara, or about 0.05 bara, or about 0.1 bara, or about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara. A disclosed method may include a hydrogen pressure of about 1.0 bara, or about 10 bara, or about 20 bara, or about 30 bara, or about 40 bara, or about 50 bara, or about 60 bara, or about 70 bara, or about 80 bara, or about 90 bara, or about 100 bara, where about includes plus or minus 5 bara. In some embodiments, in place of a hydrogenation reactor 115, a system may include a stirred-tank reactor including a suspended catalyst at a concentration ranging from about 0.01 g/L to about 100 g/L. A stirred-tank reactor may include a suspended catalyst at a concentration of about 0.01 g/L, or about 0.1 g/L, where about includes plus or minus 0.05 g/L. A stirred-tank reactor may include a suspended catalyst at a concentration of about 1.0 g/L, or about 10 g/L, or about 20 g/L, or about 30 g/L, or about 40 g/L, or about 50 g/L, or about 60 g/L, or about 70 g/L, or about 80 g/L, or about 90 g/L, or about 100 g/L, where about includes plus or minus 5 g/L.

In some embodiments, as shown in FIG. 1, once a hydrogenation reactor forms a MHCO₃ and HCOOM mixture, the mixture may be transferred to a crystallization unit 120 through a MHCO₃/HCOOM transfer line. A crystallization unit 120 may be configured to receive a MHCO₃ and HCOOM mixture through a MHCO₃/HCOOM mixture transfer line. In some embodiments, a crystallization unit 120 may be connected to a dryer/inert treatment reactor 125 through a MOOCH transfer line. A crystallization unit may be configured to separate a MHCO₃ and HCOOM mixture through fractional crystallization into a separated MHCO₃ and a separated HCOOM. In some embodiments, a crystallization unit 120 includes a container made of any material (e.g., glass, metal, plastic), a cooling element, a heating element (e.g., thermocouple), and a vacuum).

In some embodiments, as shown in FIG. 1, a HCOOM (e.g., HCOONa, HCOOK) produced by a crystallization unit 120 may be transferred to a dryer/inert treatment reactor 125 through a HCOOM transfer line. A dryer/inert treatment reactor 125 may be configured to treat a HCOOM to an inert thermal treatment with a catalytic amount of a MOH (e.g., KOH, NaOH) at a temperature ranging from about 300° C. to about 400° C. to produce a dried M₂(COO)₂. A catalytic amount of a MOH may include a range from about 1 wt % MOH to about 5 wt % MOH (wt % relative to the amount of HCOOM and MOH). In some embodiments, a dryer/inert treatment reactor 125 may be configured to treat a HCOOM to an inert thermal treatment at a temperature of about 300° C., or about 310° C., or about 320° C., or about 330° C., or about 340° C., or about 350° C., or about 360° C., or about 370° C., or about 380° C., or about 390° C., or about 400° C., or about 410° C. where about includes plus or minus 5° C. A dryer/inert treatment reactor 125 may include a container made of any substance (e.g., metal, glass, plastic), a heating element (e.g., thermocouple), and a feed line for a MOH.

As shown in FIG. 1, a disclosed system may include a polymerization reactor 130 connected to a metal ion exchange bubble column 110 and may receive a (COOMe)₂ from the metal ion exchange bubble column 110 through a (COOMe)₂ transfer line. In some embodiments, a polymerization reactor 130 may receive one or more of a (COOMe)₂ and a (COOEt)₂ from a bubble reactor 110 and convert it to a hydrogel as well. A polymerization reactor 130 may be connected to a 2nd reactant stream 135 through a 2nd reactant transfer line and may be connected to a hydrogel tank 140 through a hydrogel transfer line. In some embodiments, a polymerization reactor 130 may combine a (COOMe)₂ with a glycerine supplied by a 2nd reactant stream 135 and a catalytic amount of an acid (e.g., H₂SO₄) to produce a hydrogel and a methanol. An acid includes any known acid including nitric acid, sulfuric acid, perchloric acid, chloric acid, acetic acid, sulfurous acid, methanoic acid, phosphoric acid, nitrous acid, hydrofluoric acid, p-toluenesulfonic acid and combinations thereof. A catalytic amount of acid includes from about 0.01 wt. % to about 1.0 wt. %, by weight of the one or more of a (COOMe)₂ ora (COOEt)₂, and glycerine. A catalytic amount of an acid includes 0.01 wt. %, or about 0.05 wt. %, or about 0.1 wt. %, or about 0.25 wt. %, or about 0.5 wt. %, or about 0.75 wt. %, or about 1.0 wt. %, where about includes plus or minus 0.05 from 0.01 wt. % to 0.1 wt. % and plus or minus 0.125 from 0.1 wt. % to 1.0 wt. %. A polymerization reactor 130 may include a reaction container made from any material (e.g., a glass, a metal, a plastic) that is made of any general size (e.g., lab, pilot plant, plant) and shape, a heating element, a cooling element, a vacuum element, a mixing element (e.g., stirrer, shaker), and a pressure regulator.

As shown in FIG. 1, a system 100 may include a 2nd reactant stream 135. A 2nd reactant stream 135 may include a container made from any know material (e.g., a glass, a plastic, a metal) that may be an open container or a closed container. A 2nd reactant stream 135 may include any conveyance mechanism to facilitate a transfer of a 2nd reactant (e.g., glycerine) from a 2nd reactant stream 135 to a polymerization reactor 130 through a 2nd reactant transfer line.

In some embodiments, a system 100 includes a hydrogel tank 140 that may be configured to receive a hydrogel from a polymerization reactor 130 through a hydrogel transfer line. A hydrogel tank 140 may include a container made from any know material (e.g., a glass, a plastic, a metal) that may be an open container or a closed container.

According to some embodiments, as shown in FIG. 2, a system may include an off gas tank 245 configured to transfer a gas including a CO₂, a H₂, a CO, a N₂, and a hydrocarbon having the formula CₓH_{y}, from the off gas tank 245 to a CO converter 250 through an off gas transfer line. In some embodiments, an off gas tank 245 may be maintained at a pressure ranging from about 40 bara to about 70 bara and at a temperature ranging from about 50° C. to about 80° C. An off gas tank 245 may include a container made from any know material (e.g., a glass, a plastic, a metal)

In some embodiments, a system 200 as shown in FIG. 2 includes a CO convertor 250, which may be configured to receive an off gas from an off gas tank 245 and to transform the off gas into a H₂ and a gas including a CO₂, a N₂, and a hydrocarbon having the formula CₓH_{y}. A CO convertor 250 may produce a H₂ and may transfer it to a hydrogenation reactor 115 through a H₂ transfer line. In some embodiments, a CO Convertor 250 may produce a gas including a CO₂, a N₂, and a hydrocarbon having the formula CₓH_{y} and may transfer the gas to a metal ion exchange bubble column 110 through a CO₂, N₂, CₓH_{y} transfer line. In some embodiments, a CO convertor 250 may receive a MHCO₃ from a metal ion exchange bubble column 110 through a MHCO₃ transfer line. As shown in FIG. 2, a system 200 may include a N₂, CO₂, CₓH_{y} collector 255. A N₂, CO₂, CₓH_{y} collector 255 may be connected to a metal ion exchange bubble column 110 and may be configured to receive a gas including a N₂, a CO₂, and a CₓH_{y} from the metal ion exchange bubble column 110 through a CO₂, N₂, CₓH_{y} transfer line. A N₂, CO₂, CxHy collector 255 may include a container made from any material include a plastic, a glass, and a metal and may be of any size or shape. A N₂, CO₂, CₓH_{y} collector 255 may include an open container and a closed container. In some embodiments, a N₂, CO₂, CₓH_{y} collector 255 may include a transfer line (e.g., pipe) that may convey gas away from system 200. For example, a N₂, CO₂, CₓH_{y} collector 255 may include including a pipe line and a vehicle (e.g., transfer truck).

### Methods for Converting a CO₂ to a Hydrogel

The present disclosure, according to some embodiments, relates to a method for generating a hydrogel from a CO₂ gas stream. An exemplary pathway includes the stoichiometry shown below:

KOH+CO₂→KHCO₃ 1)

KHCO₃+H₂→HCOOK+H₂O 2)

2HCOOK→K₂(COO)₂+H₂ 3)

K₂(COO)₂+H-IE→H₂(COO)₂+K-IE 4)

H₂(COO)₂+2EtOH→Et₂(COO)₂+H₂O 5)

a Et₂(COO)₂+b CH₂OH—CHOH—CH₂OH→CxHyOz+c EtOH 6)

An exemplary pathway disclosed herein includes a method using a metal ion exchange bubble column 110 to convert a CO₂ gas into a (COOMe)₂, using a polymerization reactor 130 to convert the (COOMe)₂ to a hydrogel, using a hydrogenation reactor 115 to convert a MHCO₃ to a MHCO₃/MOOCH mixture, a crystallization unit 120 to convert the MHCO₃/MOOCH mixture to a HCOOM, and a dryer/inert treatment reactor 125 to convert the HCOOM to a M2(COO₂) that is fed to the metal ion exchange bubble column to be converted to the (COOMe)₂. According to some embodiments, the present disclosure relates to a method for generating a hydrogel from a CO₂ gas stream. A method may include a step of passing a CO₂ gas stream including a CO₂ through a water bath to produce a carbonated water. A water bath may be cooled or heated. In some embodiments, a water bath may be set at a temperature ranging from about 0° C. to about 100° C. A water bath may be set at a temperature of about 0° C., or about 10° C., or about 20° C., or about 30° C., or about 40° C., or about 50° C., or about 60° C., or about 70° C., or about 80° C., or about 90° C., or about 100° C., where about includes plus or minus 5° C. A carbonated water may be from about 1% saturated with a CO₂ to about 100% saturated with a CO₂. A carbonated water may be about 1% saturated with a CO₂, or about 10% saturated with the CO₂, or about 20% saturated with the CO₂, or about 30% saturated with the CO₂, or about 40% saturated with the CO₂, or about 50% saturated with the CO₂, or about 60% saturated with the CO₂, or about 70% saturated with the CO₂, or about 80% saturated with the CO₂, or about 90% saturated with the C_{O2}, or about 100% saturated with the CO₂, where about includes plus or minus 5% saturation.

According to some embodiments, a carbonated water may be passed through an ion exchange bubble column 110 including a M₂(COO)₂ (e.g., Na₂(COO)₂, K₂(COO)₂) to produce a (COOH)₂ and a MHCO₃ (e.g., NaHCO₃, KHCO₃). An ion exchange column may be acidic or basic. In some embodiments, a method may include a step of passing a (COOH)₂ generated in an ion exchange bubble column 110 through an activated carbon bed to produce a (COOH)₂ absorbed carbon bed. In some embodiments, a (COOH)₂ absorbed carbon bed may be from about 1% saturated with absorbed (COOH)₂ to about 100% saturated with absorbed (COOH)₂.

A (COOH)₂ absorbed carbon bed may be about 1% saturated with absorbed (COOH)₂, or about 10%, or about 20%, or about 30%, or about 40%, or about 50%, or about 60%, or about 70%, or about 80%, or about 90%, or about 100%, where about includes plus or minus 5%.

In some embodiments, a method may include a step of passing a mono-alcohol through a (COOH)₂ absorbed carbon bed to produce a one or more of a (COOCH₃)₂, a (COOEt)₂, and any alcohol based product based on the alcohol passed through the (COOH)₂ absorbed carbon bed. For example, if ethanol is used a (COOEt)₂ may be generated or if methanol is used a (COOCH₃)₂ may be generated. In some embodiments, a mono-alcohol includes methanol, ethanol, propanol, any straight chain or branched alcohol C₁-C₁₀, and combinations thereof.

According to some embodiments, a method may include forming one or more of a (COOCH₃)₂, a (COOEt)₂, and other alcohol reaction formed products by combining a (COOH)₂, an alcohol (e.g., methanol, ethanol, and a first acid catalyst (e.g., H₂SO₄) in a reactive distillation reactor at a temperature ranging from about 80° C. to about 100° C. and under atmospheric pressure. An acid includes any known acid including hydrochloric acid, nitric acid, sulfuric acid, hydrobromic acid, hydroiodic acid, perchloric acid, chloric acid, acetic acid, sulfurous acid, methanoic acid, phosphoric acid, nitrous acid, hydrofluoric acid, and combinations thereof. A catalytic amount of acid includes form about 0.01 wt. % to about 1.0 wt. %, by weight of the one or more of a (COOMe)₂ and a (COOEt)₂. A catalytic amount of an acid includes 0.01 wt. %, or about 0.05 wt. %, or about 0.1 wt. %, or about 0.25 wt. %, or about 0.5 wt. %, or about 0.75 wt. %, or about 1.0 wt. %, where about includes plus or minus 0.05 from 0.01 wt. % to 0.1 wt. % and plus or minus 0.125 from 0.1 wt. % to 1.0 wt. %. In some embodiments, an alcohol includes methanol, ethanol, propanol, any straight chain or branched alcohol C₁-C₁₀, and combinations thereof. A temperature includes about 80° C., or about 85° C., or about 90° C., or about 95° C., or about 100° C., where about includes plus or minus 2.5° C.

In some embodiments, a method may include combining a glycerine, an acid catalyst (e.g., a H₂SO₄), and one or more of a (COOCH₃)₂ and the (COOEt)₂ produced from the metal ion exchange bubble column 110 to produce a hydrogel and an alcohol (e.g., methanol, ethanol). An acid includes any known acid including hydrochloric acid, nitric acid, sulfuric acid, hydrobromic acid, hydroiodic acid, perchloric acid, chloric acid, acetic acid, sulfurous acid, methanoic acid, phosphoric acid, nitrous acid, hydrofluoric acid, and combinations thereof. A catalytic amount of acid includes form about 0.01 wt. % to about 1.0 wt. %, by weight of the one or more of a (COOMe)₂ and a (COOEt)₂. A catalytic amount of an acid includes 0.01 wt. %, or about 0.05 wt. %, or about 0.1 wt. %, or about 0.25 wt. %, or about 0.5 wt. %, or about 0.75 wt. %, or about 1.0 wt. %, where about includes plus or minus 0.05 from 0.01 wt. % to 0.1 wt. % and plus or minus 0. 125 from 0.1 wt. % to 1.0 wt. %. In some embodiments, a method may include removing the alcohol by-product through treatment with heat, a vacuum, or both to produce a substantially alcohol free hydrogel. In some embodiments, a produced hydrogel does not need to be substantially alcohol free.

In some embodiments, a method may include a step of combining a MHCO₃ produced from an ion exchange bubble column 110 with a hydrogen gas in a hydrogenation reactor 115 including a catalyst (e.g., a palladium catalyst) at a temperature ranging from about 15° C. to about 100° C. and a pressure ranging from about 0.1 bara to about 100 bara to produce a HCOOM and a MHCO₃ mixture. A method may include a catalyst including one or more of a palladium catalyst, a nickel catalyst, and a platinum catalyst. A catalyst includes, but is not limited to, a Pd/C, a Pd/Al₂O₃, a Ni/SiO₂, a Pd/theta Al₂O₃, a SiO₂/Al₂O₃, and combinations thereof. A method includes using a catalyst at a concentration ranging from about 0.1 g/100 mL of solvent (e.g., organic or inorganic) to about 5 g/100 mL of solvent. A method includes using a catalyst at a concentration of about 0.1 g/100 mL of solvent, or about 0.5 g/100 mL of solvent, or about 1.0 g/100 mL of solvent, or about 1.5 g/100 mL of solvent, or about 2.0 g/100 mL of solvent, or about 2.5 g/100 mL of solvent, or about 3.0 g/100 mL of solvent, or about 3.5 g/100 mL of solvent, or about 4.0 g/100 mL of solvent, or about 4.5 g/100 mL of solvent, or about 5.0 g/100 mL of solvent, where about includes plus or minus 0.25 g/100 mL of solvent. A hydrogenation reactor 115 may include a temperature of about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C., or about 85° C., or about 90° C., or about 95° C., or about 100° C., or about 105° C., or about 110° C., or about 115° C., or about 120° C., or about 125° C., or about 130° C., or about 135° C., or about 140° C., or about 145° C., or about 150° C., where about includes plus or minus 2.5° C. According to some embodiments, a hydrogenation reactor 115 may include a hydrogen pressure of about 0.001bara, or about 0.005 bara, or about 0.01 bara, or about 0.05 bara, or about 0.1 bara, or about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara. A disclosed method may include a hydrogen pressure of about 1.0 bara, or about 10 bara, or about 20 bara, or about 30 bara, or about 40 bara, or about 50bara, or about 60 bara, or about 70 bara, or about 80 bara, or about 90 bara, or about 100 bara, where about includes plus or minus 5 bara.

In some embodiments, a method may include a step of separating a MHCO₃ and HCOOM mixture through fractional crystallization in a crystallization unit 120 into a separated MHCO₃ (e.g. NaHCO₃, KHCO₃) and a separated HCOOM (e.g., HCOONa, HCOOK). A separated MHCO₃ may be recycled back to a hydrogenation reactor 115 and a separated HCOOM may be transferred to a dryer/inert treatment reactor 125. A crystallization unit 120 may be at a temperature ranging from about 0° C. to about 500° C. A temperature includes about 0° C., or about 50° C., or about 100° C., or about 150° C., or about 200° C., or about 250° C., or about 300° C., or about 350° C., or about 400° C., or about 450° C., or about 500° C., where about includes plus or minus 25° C. A temperature of a crystallization unit 120 may be maintained by a cooling jacket, a cooling bath, a heating jacket, a thermocouple, or any other means of temperature control. Within a crystallization unit 120, any solvent may be used including both organic (e.g., methanol, ethyl acetate, hexanes, methylene chloride) and aqueous (e.g., water) solvents. A solvent may be added to a crystallization unit from a solvent tank. According to some embodiments, no solvents are used. In some embodiments, a crystallization unit 120 may be made of any material including metal, glass, ceramic, plastic, and combinations thereof. A crystallization unit 120 may separate a MHCO₃ (e.g. NaHCO₃, KHCO₃) and a HCOOM by turning one into a solid while one remains a liquid or by forming separate solids of each.

According to some embodiments, a method may include a step of treating a HCOOM to an inert thermal treatment in a dryer/inert treatment reactor 125 with a catalytic amount of a MOH (e.g., KOH, NaOH) at a temperature of ranging from about 100° C. to about 400° C. to produce a hydrogen gas and a dried M₂(COO)₂ (e.g., K₂(COO)₂, Na₂(COO)₂. A temperature includes about 100° C., or about 120° C., or about 140° C., or about 160° C., or about 180° C., or about 200° C., or about 220° C., or about 240° C., or about 260° C., or about 280° C., or about 300° C., or about 320° C., or about 340° C., or about 360° C., or about 380° C., or about 400° C., where about includes plus or minus 10° C. In some embodiments, a hydrogen gas may be transferred to a hydrogenation reactor 115 and a dried M₂(COO)₂ may be transferred to an ion exchange bubble column 110. A catalytic amount of a MOH may include the MOH at a concentration ranging from about 0.01 mmol to about 1 mmol. A MOH concentration includes about 0.01mmol, or about 0.1 mmol, or about 0.2 mmol, or about 0.3 mmol, or about 0.4 mmol, or about 0.5 mmol, or about 0.6 mmol, or about 0.7 mmol, or about 0.8 mmol, or about 0.9 mmol, or about 1 mmol, where about includes plus or minus 0.05 mmol. A MOH includes NaOH, KOH, or NH₄OH, and combinations thereof. An inert thermal treatment may be conducted in the presence of an inert gas including nitrogen, argon, helium, and combinations thereof. An inert gas may be introduced into a dryer/inert treatment reactor 125 from one or more inert gas tanks through a gas pressure regulator. In some embodiments, a method includes combining a M₂(COO)₂ with a MOH in a reactive multi-stage forced cooling crystallization system to produce a M₂(COO)₂ and a MOH. A M₂(COO₎₂ may be combined with an acid (e.g., H₂SO₄) in a reactive crystallization system to produce a (COOH)₂ and a M₂SO₄. A reactive crystallization system may be at a temperature ranging from about 40° C. to about −200° C. A temperature includes about 40° C., or about 20° C., or about 0° C., or about −20° C., or about −40° C., or about −60° C., or about −80° C., or about −100° C., or about −120° C., or about −140° C., or about −160° C., or about −180° C., or about −200° C., where about includes plus or minus 10° C. A temperature of a reactive crystallization system may be maintained by a cooling jacket, a cooling bath, a heating jacket, a thermocouple, or any other means of temperature control. Within a reactive crystallization system, any solvent may be used including both organic (e.g., methanol, ethyl acetate, hexanes, methylene chloride) and aqueous (e.g., water) solvents. A solvent may be added to a reactive crystallization system from a solvent tank. According to some embodiments, no solvents are used. In some embodiments, a reactive crystallization system may be made of any material including metal, glass, ceramic, plastic, and combinations thereof. A reactive crystallization system may separate a (COOH)₂ and a M₂SO₄ by turning one into a solid while one remains a liquid or by forming separate solids of each.

In some embodiments, a method includes a step of passing a CO₂ gas stream including a CO₂ through an absorption column including a MOH (e.g., NaOH, KOH) to produce a MHCO₃ (e.g., NaHCO₃, KHCO₃) and an off gas. A separated MHCO₃ may be recycled back to a hydrogenation reactor 115 and a separated HCOOM.

The present disclosure further relates, according to some embodiments, to methods of using a hydrogel produced from a CO₂ gas stream to sequester carbon in soils. A hydrogel may be combined with a soil to produce a soil-based hydrogel product. In some embodiments, a bentonite may be added to a soil-based hydrogel products at a weight ranging from about 1 wt. % to about 50 wt. %, by weight of the soil-based hydrogel product, which may produce a dry powder. A soil-based hydrogel product may include a bentonite at about 1 wt. %, or about 10 wt. %, or about 20 wt. %, or about 30 wt. %, or about 40 wt. %, or about 50 wt. %, where about includes plus or minus 5 wt. %, by weight of the soil-based hydrogel product. A formed powder product may desirably be formed into tablets, pellets, and other shapes and sizes.

According to some embodiments, a disclosed method for generating an ester from a CO₂ gas stream includes a step of converting a CO₂ from the CO₂ gas stream into a (COOH)₂, and combining the (COOH)₂, a CH₃CH₂OH, and a first acid catalyst comprising a H₂SO₄ at a temperature ranging from about 80° C. to about 100° C. and under atmospheric pressure to produce the carbon sequestering agent containing a (COOEt)₂. The obtained ester may be considered an intermediate product.

A disclosed method may include a step of combining an ester, a glycerine, and a second acid catalyst (e.g., H₂SO₄) at a temperature ranging from about 100° C. to about 200° C. to produce a poly-ester, preferably in the form of hydrogel, and an ethanol. A temperature includes about 100° C., or about 110° C., or about 120° C., or about 130° C., or about 140° C., or about 150° C., or about 160° C., or about 170° C., or about 180° C., or about 190° C., or about 200° C., where about includes plus or minus 5° C. In some embodiments, a disclosed method may include a step of combining an ester, a glycerine, and a second acid catalyst at a pressure ranging from about 0.1 bara to about 100 bara to produce a polyester which preferably in the form of a hydrogel, and an ethanol. A pressure includes about 0.001 bara, or about 0.005 bara, or about 0.01 bara, or about 0.05 bara, or about 0.1 bara, or about 0.5 bara, or about 1.0 bara, where about includes plus or minus 0.0025 bara in between 0.001 bara and 0.01 bara, plus or minus 0.025 in between 0.01 and 0.1 bara, and plus or minus 0.25 in between 0.1 bara and 1.0 bara.

A disclosed method may include a hydrogen pressure of about 1.0 bara, or about 10 bara, or about 20 bara, or about 30 bara, or about 40 bara, or about 50 bara, or about 60 bara, or about 70 bara, or about 80 bara, or about 90 bara, or about 100 bara, where about includes plus or minus 5 bara.

In an embodiment the polyester is in the form of a hydrogel.

Disclosed embodiments also include methods of supplementing a soil with a carbon sequestering agent, such as a hydrogel, as described herein. A method may include a step of combining at least a hydrogel with a soil, where the hydrogel comprises a polyester which includes one or more of a (COOCH₃)₂ and a (COOEt)₂. A hydrogel may have a structure according to Formula I.

In some embodiments, a disclosed polyester hydrogel may decompose into CO₂, water, small organic building blocks (e.g., organic carboxylic acids), and other environmentally benign products. Decomposition of a polyester hydrogel may be facilitated by microbes (e.g., bacteria, fungi), heat, water, sunlight, weather, cold, acids provided by the environment (e.g., acid rain), and combinations thereof. In some embodiments, a polyester hydrogel may autonomously decompose. A disclosed polyester hydrogel may retain, absorb, or release carbon dioxide while decomposing over time. For example, throughout a decomposition life cycle, a polyester hydrogel may absorb and retain CO₂ received from the environment. A polyester hydrogel may also release CO₂ to surrounding plant life to serve as a carbon nutrient source.

The present invention also relates to the following preferred embodiments:

In an embodiment, the present invention relates to a method for converting a CO₂ gas stream comprising a CO₂ into an ester, the method comprising:
(b) converting the CO₂ into a (COOH)₂;
(c) passing the (COOH)₂ and a mono-alcohol through an activated carbon bed to generate an ester, wherein the ester comprises two or more of a (COOCH3)₂ and a (COOEt)₂.

In an embodiment, the method according to the present invention further comprises:
(c) combining a glycerine, an acid catalyst comprising a H₂SO₄, and the ester to produce a hydrogel and a mono-alcohol, wherein the hydrogel has a structure according to Formula I.

In an embodiment, converting the CO₂ into the (COOH)₂ according to the method according to the present invention comprises:
passing the CO₂ through a water bath to produce a carbonated water; and
passing the carbonated water through a metal ion exchange bubble column comprising a M₂(COO)₂ to produce the (COOH)₂ and a MHCO₃,

In an embodiment, the method according to the present invention further comprises:
(d) combining a glycerine, an acid catalyst comprising a H₂SO₄, and the ester to produce a hydrogel and a mono-alcohol, wherein the hydrogel has a structure according to Formula I.

In an embodiment, the method according to the present invention further comprises:
combining the MHCO₃ produced from the metal ion exchange bubble column with a hydrogen gas in a hydrogenation reactor comprising a palladium catalyst at a temperature ranging from about 15° C. to about 100° C. and a pressure of about 0.1 bara to about 100 bara to produce a mixture comprising HCOOM and MHCO₃, wherein HCOOM comprises one or more of CHOOK and HCOONa.

In an embodiment, the method according to the present invention further comprises:
separating the mixture through fractional crystallization in a crystallization unit into a separated MHCO₃ and a separated HCOOM.

In an embodiment, the method according to the present invention further comprises:
feeding the separated MHCO₃ into the hydrogenation reactor.

In an embodiment, the method according to the present invention further comprises:
treating the separated HCOOM with a catalytic amount of MOH at a temperature of ranging from about 100° C. to about 400° C. to produce a hydrogen gas and a dried M₂(COO)₂, wherein MOH comprises one or more of KOH and NaOH, and wherein the dried M₂(COO)₂ comprises one or more of K₂(COO)₂ and Na₂(COO)₂.

In an embodiment, the method according to the present invention further comprises at least one of:
transferring the hydrogen gas to the hydrogenation reactor; and
transferring the dried M₂(COO)₂ to the metal ion exchange bubble column.

A further embodiment of the present invention relates to a system for generating a carbon sequestering agent from a CO₂ gas stream comprising a CO₂, the system comprising:
(b) a CO₂ conversion unit configured to convert the CO₂ into a (COOH)₂;
(c) an activated carbon bed configured to receive the (COOH)₂ and a mono-alcohol to generate the ester, wherein the carbon sequestering agent comprises two or more of a (COOCH3)₂ and a (COOEt)₂.

In an embodiment, the CO₂ conversion unit of the system according to the present invention is configured to combine the CO₂ with a M₂(COO)₂ to produce a (COOH)₂ and a MHCO₃, wherein the MHCO₃ comprises one or more of KHCO₃ and NaHCO₃, and wherein M₂(COO)₂comprises one or more of K₂(COO)₂ and Na₂(COO)₂.

In an embodiment, the system according to the present invention further comprises a polymerization reactor configured to receive the ester and combine the ester with a glycerine and an acid catalyst comprising a H₂SO₄ to produce a polyester and an ethanol, wherein the polyester has a structure according to Formula I.

In an embodiment, the system according to the present invention further comprises:
(c) a hydrogenation reactor connected to the CO₂ conversion unit and configured to receive the MHCO₃ from the CO₂ conversion unit and to combine the MHCO₃ with a hydrogen gas and a palladium catalyst at a temperature ranging from about 35° C. to about 80° C. and a pressure ranging from about of 1 bara to about 30 bara to produce a mixture comprising MHCO₃ and HCOOM, wherein HCOOM comprises one or more of HCOOK and HCOONa.

In an embodiment, the system according to the present invention further comprises:
(d) a crystallization unit configured to receive the mixture from the hydrogenation reactor and to separate the mixture through into a separated MHCO₃ and a separated HCOOM.

In an embodiment, the system according to the present invention further comprises:
(e) a dryer/inert treatment reactor configured to receive the separated HCOOM from the crystallization unit and to treat the separated HCOOM with a catalytic amount of KOH at a temperature of ranging from about 100° C. to about 400° C. to produce a hydrogen gas and a dried M₂(COO)₂, wherein M₂(COO)₂ comprises a K₂(COO)₂ and Na₂(COO)₂.

A further aspect of the present invention relates to a method for generating a carbon sequestering agent, preferably in the form of an hydrogel from a CO₂ gas stream, the method comprises:
(b) converting a CO₂ from the CO₂ gas stream into a (COOH)₂; and
(c) combining the (COOH)₂, a CH₃CH₂OH, and a first acid catalyst comprising a H₂SO₄ at a temperature ranging from about 80° C. to about 100° C. and under atmospheric pressure to produce an ester comprising a (COOEt)₂.

In an embodiment, the method according to the present invention further comprises:
(d) combining the ester comprising (COOEt)₂, a glycerine, and a second acid catalyst comprising a H₂SO₄ at a temperature in the range of 120 to 170° C. and a pressure ranging from about 0.3 bara to about 1 bara to produce the hydrogel and an ethanol, wherein the hydrogel has a structure according to Formula I.

A further aspect of the present invention relates to a method of supplementing a soil, the method comprises:
combining at least one of a carbon sequestering agent and a hydrogel with the soil, wherein the carbon sequestering agent comprises two or more of a (COOCH₃)2 and a (COOEt)₂, and wherein the hydrogel has a structure according to Formula I.

In the context of the present invention “a polyester according to the present invention” is a polyester obtainable, preferably prepared, from an oxalic acid or an oxalic acid ester. Preferably, a polyester according to the present invention is a branched polyester. Preferably, the polyester, in particular branched polyester, according to the present invention is prepared from an oxalic acid or oxalic acid ester, in particular an oxalic acid ester and a polyol, preferably a polyol with at least 3 hydroxygroups, in particular glycerine. Preferably, a polyester according to the present invention is poly (glycerol oxalate) (also termed to be PGO or Shell gel). Preferably, a polyester according to the present invention is a hydrogel according to the present invention.

In the context of the present invention, the terms “a polyester according to the present invention” or “a polyester having the structure according to Formula I, II, III or IV” are meant to refer to a polyester having the structure according to Formula I, II, III or IV with n and/or m each ranging independently from 1 to 5250, preferably 1 to 5000, preferablywith n and/or m each ranging independently from 500 to 4500, preferably 1000 to 4000, preferably 1500 to 3500 or preferably 2000 to 3000. In an embodiment of the present invention n and/or m is 1. In an embodiment of the present invention n and/or m each ranging independently from 250 to 750. In an embodiment of the present invention n and/or m each ranging independently from 750 to 1250. In an embodiment of the present invention n and/or m each ranging independently from 1250 to 1750. In an embodiment of the present invention n and/or m each ranging independently from 1750 to 2250. In an embodiment of the present invention n and/or m each ranging independently from 2250 to 2750. In an embodiment of the present invention n and/or m each ranging independently from 2750 to 3250. In an embodiment of the present invention n and/or m each ranging independently from 3250 to 3750. In an embodiment of the present invention n and/or m each ranging independently from 3750 to 4250. In an embodiment of the present invention n and/or m each ranging independently from 4250 to 4750. In an embodiment of the present invention n and/or m each ranging independently from 4750 to 5250.

In the context of the present invention the integers “n” and “m” indicating in the Formula I, Formula II, Formula III and Formula IV the number of bracketed building blocks of the polyester according to the present invention are independently from each other applicable to any of the Formula I, Formula II, Formula III, and Formula IV.

In the context of the present invention a “polyol” is an organic molecule with more than one alcohol group. Preferably, a polyol according to the present invention has at least two, preferably at least three alcohol groups. Preferably polyols according to the present invention are polyols of bio-origin, in particular biowaste polyols. In the context of the present invention “GPC” refers to gel permeation chromatography. Preferably, GPC is a method to analyse the average molecular weight of compounds, in particular polymers, in a sample, in particular the relative molecular weight and/or the distribution of molecular weights of the compounds, in particular polymers, in the sample.

Preferably, the GPC measurements of the present invention are carried out in DMF. Preferably, 13.2 mg of the polyester according to the present invention is mixed with 4.0 mL DMF (resulting concentration 3.3 mg/mL) and the solution is shaken at 400 rpm for 3 hours and then heated to 50° C. for one hour. Preferably, the following instruments and settings are used for the GPC investigations: The measurement is performed on a Viscotek GPCmax VE 2001 equipped with a Guard-CLM3008 precolumn and a GMHHR-N-18055 column (range: 1000-400 000 g/mol). Preferably, the eluent used is DMF with an addition of 10 mM lithium bistrifluoromethylsulfonylimide (Li(Tf2N)) with a flow of 1 mL/min at 60° C. Preferably, the injection volume is 100 μL. Preferably, the detector used is a VE 3580 RI detector from Viscotek at 35° C.

Preferably, for molecular weight ranges of at least 1050 g/mol of the present polyesters, GPC is used to analyse the molecular weight of the present polyesters. Preferably, external calibration for the GPC is conducted with a poly (styrene) (PS) standard in a molecular weight range of 1050 to 170 000 g/mol of the present polyesters. Preferably, for molecular weight ranges below 1050 g/mol of the present polyesters, the method ESI-TOF-MS is used to analyse the molecular weight of the present polyesters.

In the context of the present invention, “ESI-TOF-MS” refers to electrospray ionization time-of-flight mass spectrometry. Preferably, ESI-TOF-MS is a method to analyse the molecular weight of compounds, in particular ions of compounds, in a sample. Preferably, the solvent used for the ESI-TOF-MS measurements is hexafluoroisopropanol. Preferably, the sample is prepared at a concentration of 0.65 mg/mL (stirred for 24 h at 400 rpm at room temperature), filtered and then measured. Preferably, ESI-TOF-MS spectra are performed on a Bruker Daltonics microTOF by direct injection (180 μL h−1) with an accelerating voltage of 4.5 kV. Preferably, the spectra obtained are analysed using Bruker Daltonics ESI compass 1.3 for microTOF (Data Analysis 4.0).

In the context of the present invention, the liquid hourly space velocity (LHSV) is calculated using the following formula:

LHSV=reaction volumetric feed liquid flow rate/reaction volume.

Preferably, the reaction volume is calculated using the following formula:

Volumetric flow rate of reactants/volume of reactor.

In the context of the present invention, “combining” is meant to refer to a reacting of one component, in particular one reactant, with another component to produce at least one product component. Preferably “combining” is meant to refer to a reacting of at least two components with each other, in particular reactants, to produce at least one product component. Preferably, “combining” is meant to refer to a reaction of at least two components, in particular reactants, optionally with at least one catalyst, to produce at least one product component, preferably in one reaction step.

In an embodiment, the “combining” is conducted in the presence of at least one solvent system, in particular at least one solvent, and optionally at least one catalyst, preferably both of them. In the context of the present invention, “converting” is meant to refer to a conversion of at least one component, in particular reactant, preferably at least two components, in particular reactants, optionally with at least one catalyst to at least one product component, preferably two product components.

In an embodiment, the “converting” is conducted in the presence of at least one solvent system, in particular at least one solvent and optionally at least one catalyst, preferably both of them.

In the context of the present invention, “sequestering agent” is a polyester according to the present invention, in particular a hydrogel, and the decomposition products of the polyester, in particular the ester used for producing the polyester, water, CO₂, other enviromentially benign products and/or combinations thereof. Preferably, a sequestering agent is a polyester according to the present invention, in particular a hydrogel.

In the context of the present invention, “environmentally benign products” are products which do not harm the environment, for example due to toxicity.

In the context of the present invention, a “first acid catalyst” is at least one Brønsted acid and/or Lewis acid. Preferably, the first acid catalyst is able to catalyse an esterification reaction. Preferably, the first acid catalyst is H₂SO₄.

In the context of the present invention, a “second acid catalyst” is at least one Brønsted acid and/or Lewis acid. Preferably, the second acid catalyst is able to catalyse a polyesterfication reaction. Preferably, the second acid catalyst is selected from the group consisting of H₂SO₄, Sb₂O₃, SnCl₂, titanium isopropoxide and p-toluenesulfonic acid.

In the context of the present invention, “M” is a cation, preferably a metal, in molecular formula of the present invention, for example M₂(COO)₂, M(COO)₂, MHCO₃, HCOOM, MOH or M(OH)₂, preferably an alkali or alkaline earth metal, preferably alkali metal. Preferably, the “M” in molecular formula of the present invention is Na, K, or Ca, preferably Na or K. In an embodiment the “M” in MOH is Na, K, Ca or NH₄.

In the context of the present invention, a “CO₂ gas stream” is preferably referring to a gaseous medium, in particular a gas comprising CO₂. Preferably, the gaseous medium is in flow, that means is a stream.

In an embodiment, the gaseous medium comprises 20 to 100 Vol.-%, in particular 50 to 100 Vol.-%, in particular 80 to 100 Vol.-%, in particular 20 Vol.-%, in particular 50 Vol.-%, in particular 80 Vol.-%, in particular 100-Vol.-% CO₂. In an embodiment, the gaseous medium consists of CO₂.

In the context of the present invention “hydrostability” is meant to refer to the stability against hydrolysis of polyesters, in particular hydrogels. Preferably, the degredation of polyesters, in particular hydrogels, can be characterised based on its hydrostability. Preferably, the hydrostability is calculated according to the following equation:

$$Δ m = \frac{initial polyester mass ( g ) - dry polyester mass ( g )}{initial polyester mass ( g )}$$

In the context of the present invention, the WHC determined according to method 1 of the example section is calculated according to the following equation:

$$WHC = \frac{Wet sample weight - oven dry sample weight}{Oven dry sample weight}$$

In the context of the present invention, the term “bara” refers to the absolute pressure in bar. Further preferred embodiments are the subject matter of dependent claims.

The present invention is explained in more detail by the following examples and the accompanying figures.

By way of this reference the appended claims form an integral part of this disclosure.

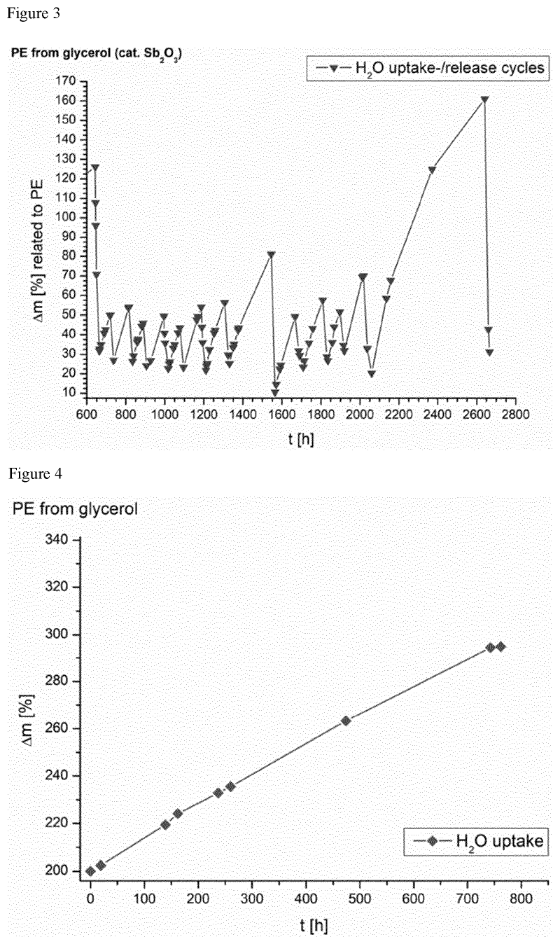

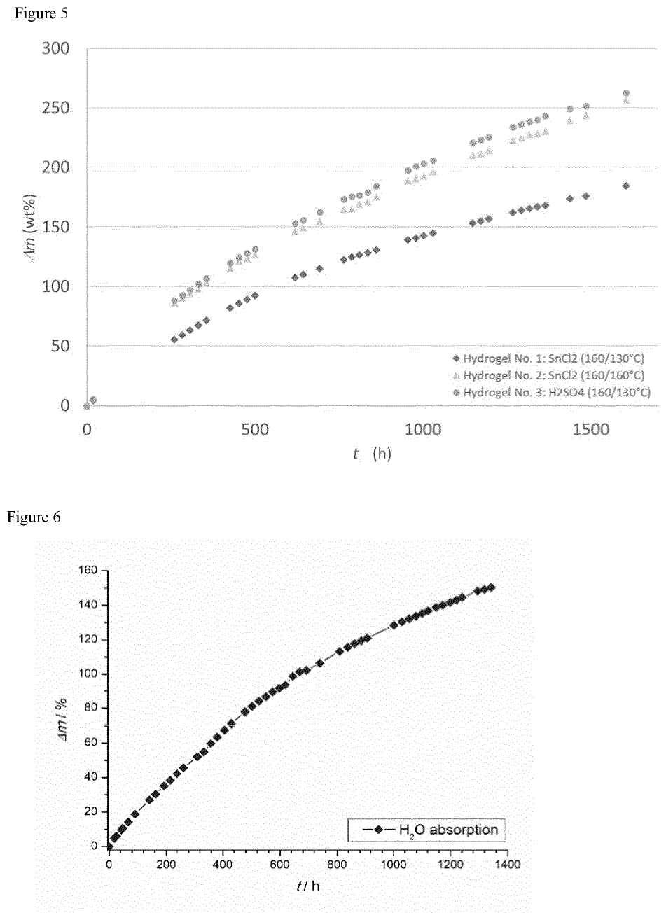

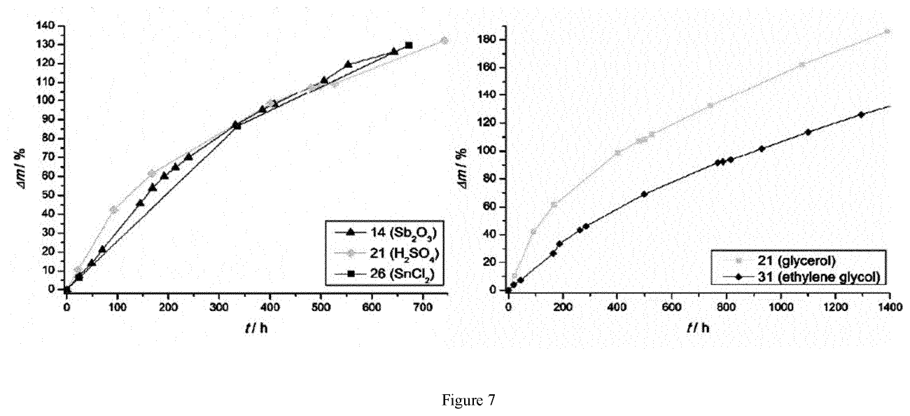

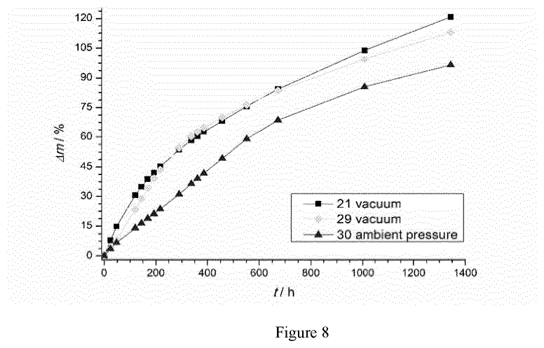

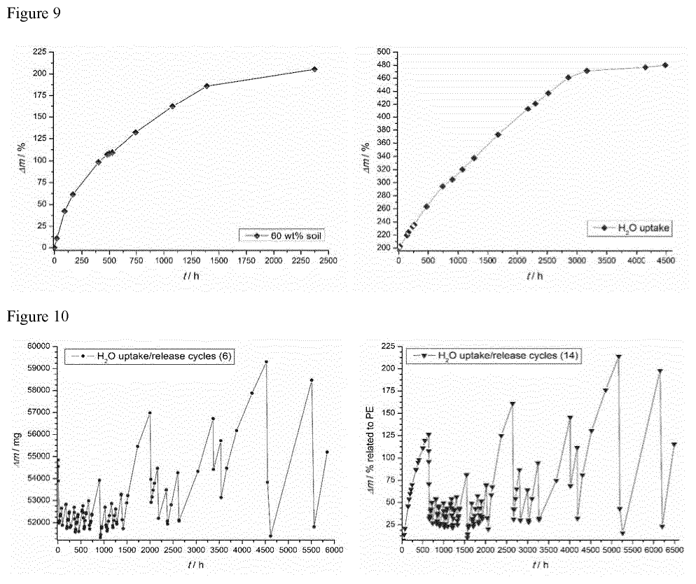

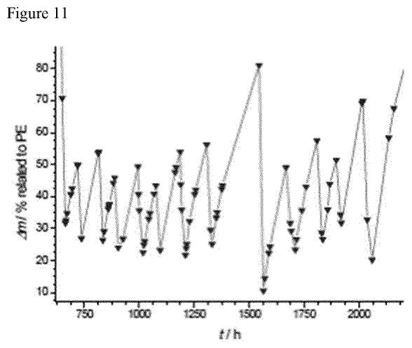

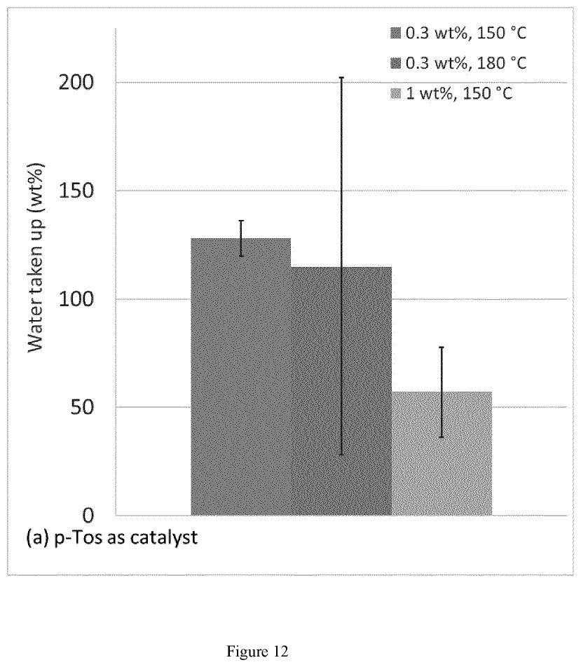

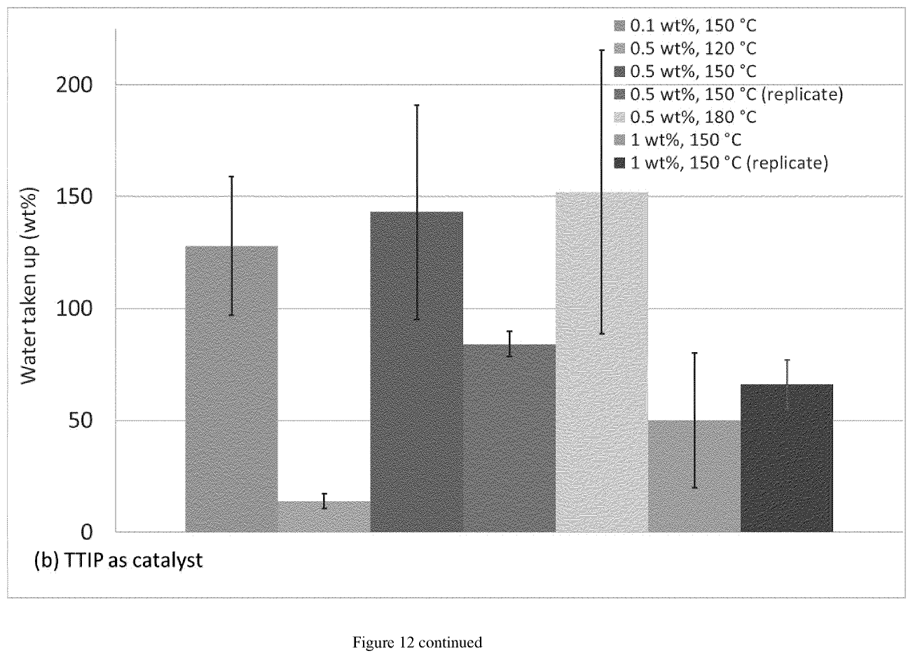

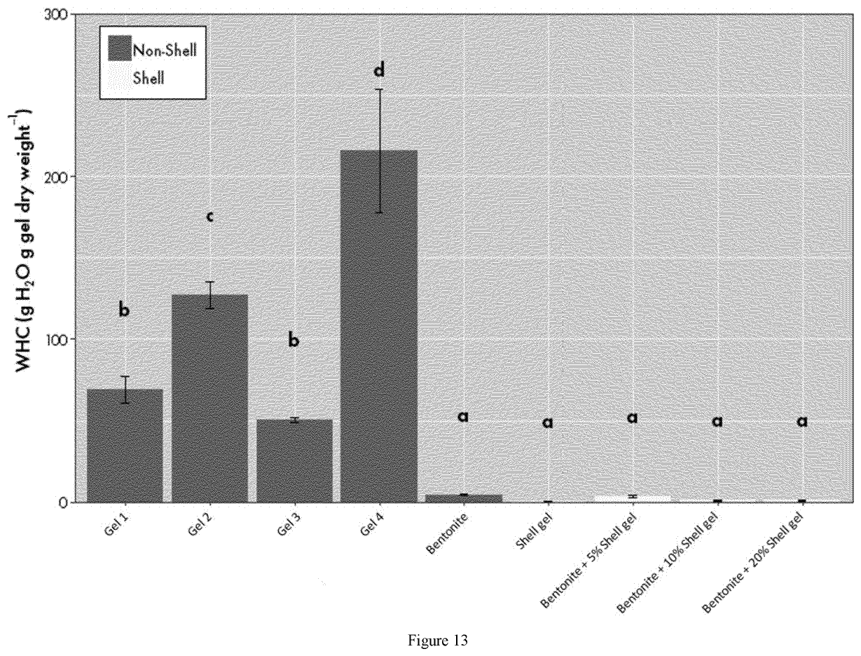

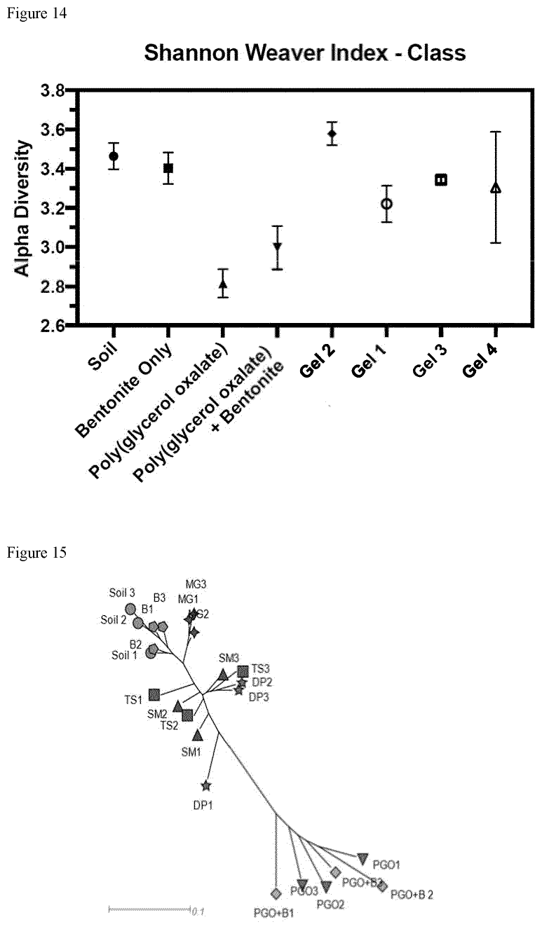

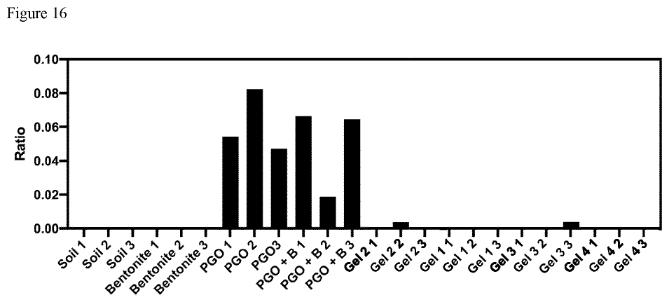

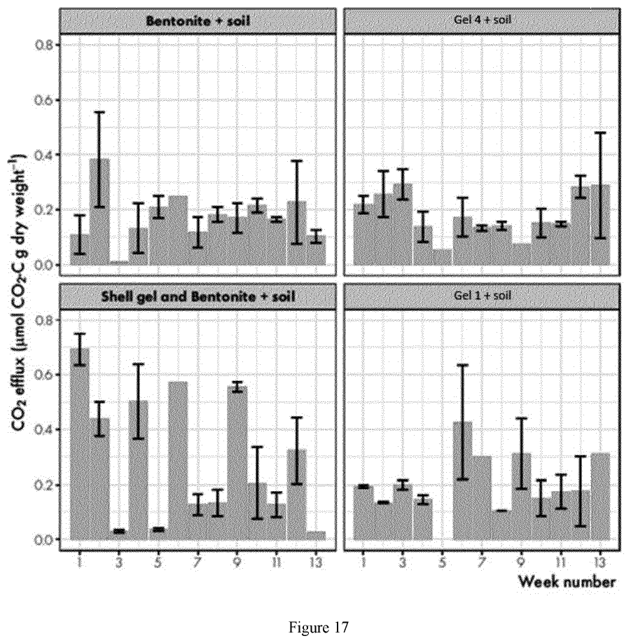

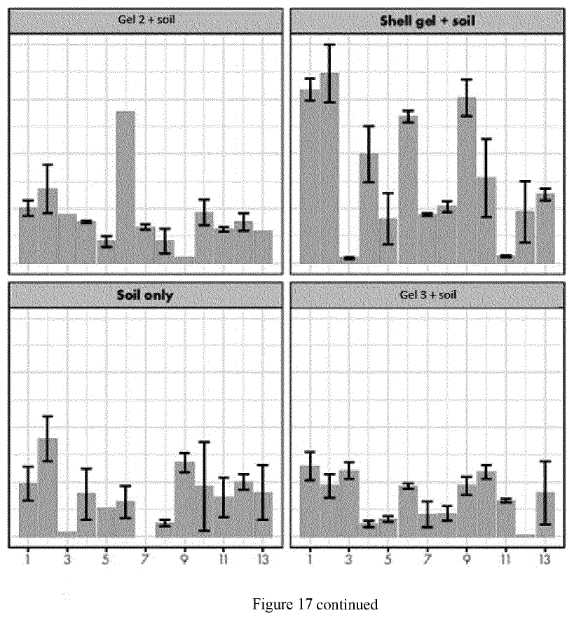

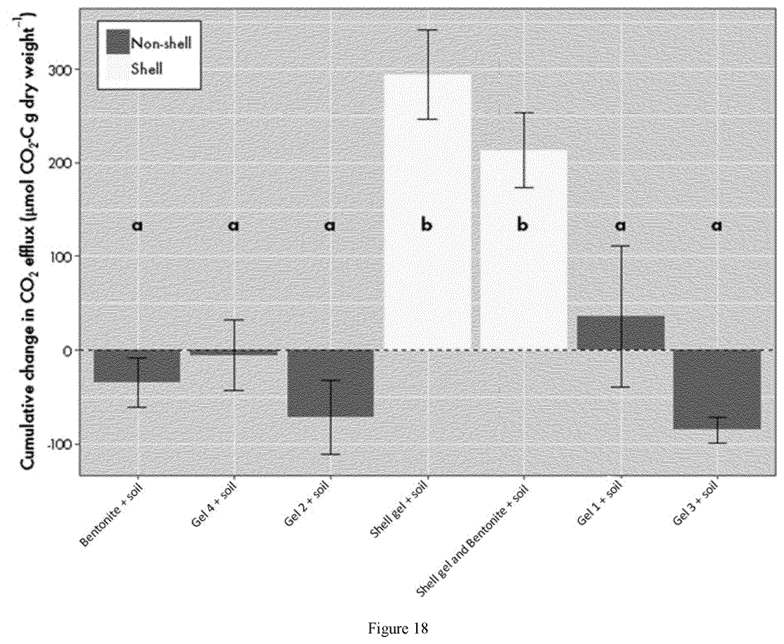

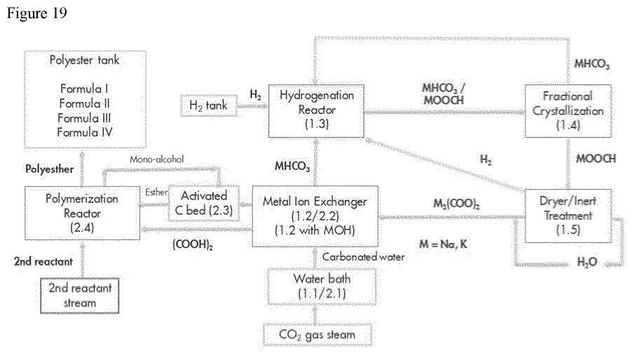

Some embodiments of the disclosure may be understood by referring, in part, to the present disclosure and the accompanying drawings.
FIG. 1 is a diagram of a system for generating a hydrogel from a CO₂ gas stream;
FIG. 2 is a diagram of a system for generating a hydrogel from a CO₂ gas stream including an off gas tank, a CO Convertor, and a N₂, CₓH_{y} collector;
FIG. 3 is a plot showing water uptake and release cycles from 600 hours to 2,800 hours, according to a specific example embodiment of the disclosure;
FIG. 4 is a plot showing a water uptake cycle from 0 hours to 800 hours, according to a specific example embodiment of the disclosure;
FIG. 5 is a diagram showing the time dependence of the mass difference for poly (glycerol oxalate) samples (hydrogel No. 1 to 3) at ambient temperature. Sample J1-A (Hydrogel No. 1, diamond) was synthesised with SnCl₂ as second acid catalyst at reaction temperatures between 160 and 130° C., sample J2-A (Hydrogel No. 2, triangle) was synthesised with SnCl₂ as second acid catalyst catalyst at a reaction temperature of 160° C. and sample J3-A (Hydrogel No. 3, circle) was synthesised with H₂SO₄ as second acid catalyst at reaction temperatures between 160 and 130° C.,
FIG. 6 is a plot showing the time dependence of the mass difference for polyester samples 6 of the present invention over a timeframe from 0 to 1400 hours (left) at ambient temperature;
FIG. 7 are plots showing the time dependence of the mass difference for polyester samples of the present invention and a poly (ethylene oxalate) control: left plot: sample 14 (triangle), sample 21 (diamond) and sample 26 (rectangle) at ambient temperature over a timeframe from 0 to 700 hours; and right plot: sample 21 (rectangle) and control 31: poly (ethylene oxalate) (diamond) at ambient temperature over a timeframe from 0 to 1400 hours;
FIG. 8 is a plot showing the time dependence of the mass difference for polyester sample 21: catalyst: H₂SO₄ vacuum step conducted (rectangle); sample 29: catalyst: H₂SO₄, oxalic acid in MeOH vacuum step conducted (diamond); and sample 30: catalyst: H₂SO₄, oxalic acid in water, before vacuum step conducted (triangle); over a timeframe from 0 to 1400 hours;
FIG. 9 are plots showing the time dependence of the mass difference for sample 21±60 wt. % soil over a timeframe from 0 to 2500 hours at ambient temperature (left) and sample 21±200 wt. % H₂O (right) over a timeframe from 0 to 4500 hours at ambient temperature;
FIG. 10 are plots showing the time dependence of the mass difference of water uptake/release cycles for sample 6 at alternating temperatures of 25° C. and 35° C. over a timeframe from 0 to 6000 hours (left) and sample 14 (right) at alternating temperatures of 25° C. and 35° C. over a timeframe from 0 to 6500 hours;
FIG. 11 is a plot showing the enlarged section of the water uptake/release cycle of sample 14 of FIG. 10;
FIG. 12 is a diagram showing the WHC comparison of samples synthesised with (a) p-toluenesulfonic acid (FIG. 12 left) and (b) titanium isopropoxide (FIG. 12 right) as second acid catalysts at different reaction conditions, carried out in triplicate. FIG. 12 left shows from left to right the WHC of polyester samples according to the present invention synthesised with 0.3 wt. % of p-toluenesulfonic acid as second acid catalyst at 150° C. (first bar); 0.3 wt. % of p-toluenesulfonic acid as second acid catalyst at 180° C. (second bar), and 1.0 wt. % of p-toluenesulfonic acid as second acid catalyst at 150° C. (third bar). FIG. 12 right shows from left to right the WHC of polyester samples according to the present invention synthesised with 0.1 wt. % of titanium isopropoxide as second acid catalyst at 150° C. (first bar); 0.5 wt. % of titanium isopropoxide as second acid catalyst at 120° C. (second bar); 0.5 wt. % of titanium isopropoxide as second acid catalyst at 150° C. (third bar); 0.5 wt. % of titanium isopropoxide as second acid catalyst at 150° C. being a replicate of the reaction conditions of the third bar (fourth bar); 0.5 wt. % of titanium isopropoxide as second acid catalyst at 180° C. (fifth bar); 1.0 wt. % of titanium isopropoxide as second acid catalyst at 150° C. (sixth bar), and 1.0 wt. % of titanium isopropoxide as second acid catalyst at 150° C. being a replicate of the reaction conditions of the sixth bar (seventh bar);
FIG. 13 is a diagram showing the water holding capacity (WHC) for commercially available hydrogels and Bentonite (termed Non-Shell in FIG. 13; grey bars), a polyester according to the present invention and agricultural compositions according to the present invention (termed Shell gel in FIG. 13; white bars), in particular bars from left to right: Soil Moist, Miracle Gro, Tera Sorb, Diaper polymer, Bentonite, Shell gel (polyester according to the present invention), Bentonite+5% Shell gel, Bentonite+10% Shell gel and Bentonite+20% Shell gel (agricultural compositions according to the present invention). Different letters indicate significant differences (p<0.05) between treatments in terms of the WHC, and no significant differences where letters are the same;
FIG. 14 is a diagram comparing the alpha diversity of the microbiome of soil only and samples of soil containing polyesters according to the present invention, Bentonite and commercially available hydrogels, in particular from left to right: Soil, Bentonite, polyester according to the present invention (Poly (glycerol oxalate), agricultural composition according to the present invention (Poly (glycerol oxalate)+Bentonite), Miracle Gro, Soil Moist, Terra Sorb and Diaper Polymer;
FIG. 15 is a diagram comparing the beta diversity of the microbiome of soil only (soil 1, soil 2, soil 3, circles) and samples of soil containing polyesters according to the present invention (triangles with tip downside, PGO1, PGO2, PGO3), agricultural compositions according to the present invention (PGO+B1, PGO+B2, PGO+B3, diamonds), Bentonite (Bentonite, B1, B2, B3, pentagons) and commercially available hydrogels (Miracle Gro, MG1, MG2, MG3, stars with four tips; Terra Sorb, TS1, TS2, TS3, rectangles; Soil Moist, SM1, SM2, SM3, triangles with tip upside and Diaper Polymer, DP1, DP2, DP3, stars with five tips);
FIG. 16 is a diagram showing the fungal to bacteria ratio of the microbiome of soil only and samples of soil containing polyesters according to the present invention, agricultural compositions according to the present invention, Bentonite and commercially available hydrogels, in particular from left to right: Soil 1, Soil 2, Soil 3, Bentonite 1, Bentonite 2, Bentonite 3, polyester 1 according to the present invention (PGO 1), polyester 2 according to the present invention (PGO 2), polyester 3 according to the present invention (PGO 3), agricultural composition 1 according to the present invention (PGO+B1), agricultural composition 2 according to the present invention (PGO+B2), agricultural composition 3 according to the present invention (PGO+B3), Miracle Gro 1, Miracle Gro 2, Miracle Gro 3, Soil Moist 1, Soil Moist 2, Soil Moist 3, Terra Sorb 1, Terra Sorb 2, Terra Sorb 3, Diaper Polymer 1, Diaper Polymer 2 and Diaper Polymer 3;
FIG. 17 are diagrams showing the weekly soil respiration assays of soil only and samples of soil containing polyesters according to the present invention (Shell gel, PGO), agricultural compositions according to the present invention (PGO with bentonite), Bentonite and commercially available hydrogels for a duration of 13 weeks, in particular FIG. 17 a) shows Bentonite and soil, FIG. 17 b) shows Diaper polymer and soil, FIG. 17 c) shows Miracle Gro and soil, FIG. 17 d) shows polyester according to the present invention and soil, FIG. 17 e) shows agricultural composition according to the present invention and soil, FIG. 17 f) shows Soil Moist and soil, FIG. 17 g) shows soil only and FIG. 17 h) shows Terra Sorb and soil;
FIG. 18 is a diagram showing the cumulative gel biodegradation rates of samples of soil containing polyesters according to the present invention and agricultural compositions (termed Shell gel, PGO and Shell gel and Bentonite; white bars), Bentonite and commercially available hydrogels (grey bars) calculated from 13 weeks of soil lab incubations at 65% WHC and 20° C., in particular bars from left to right: Bentonite and soil, Diaper polymer and soil, Miracle gro and soil, polyester according to the present invention and soil (Shell gel, PGO), agricultural composition according to the present invention and soil (Shell gel and Bentonite), Soil Moist and soil and Terra Sorb and soil,
FIG. 19 is a diagram schematically showing a process for generating a polyester, in particular a hydrogel, according to the present invention from CO₂ and
FIG. 20 is a diagram showing the hydrostability of polyester samples according to the present invention synthesised with (a) p-toluenesulfonic acid (p-Tos) (FIG. 21 left), and (b) titanium isopropoxide (TTIP) (FIG. 21 right) as catalyst at different reaction conditions.
FIG. 21 left shows from left to right the hydrostability of polyester samples according to the present invention synthesised with 0.3 wt. % of p-toluenesulfonic acid as second catalyst at 150° C. (first bar) and with 0.3 wt. % of p-toluenesulfonic acid as second catalyst at 180° C. (second bar). FIG. 21 right shows from left to right the hydrostability of polyester samples according to the present invention synthesised with 0.1 wt. % of titanium isopropoxide as second catalyst at 150° C. (first bar); 0.5 wt. % of titanium isopropoxide as second catalyst at 120° C. (second bar); 0.5 wt. % of titanium isopropoxide as second catalyst at 150° C. (third bar); 0.5 wt. % of titanium isopropoxide as second catalyst at 150° C. (fourth bar) being a replicate of the conditions of the third bar; 0.5 wt. % of titanium isopropoxide as second catalyst at 180° C. (fifth bar); 1 wt. % of titanium isopropoxide as second catalyst at 150° C. (sixth bar); 1 wt. % of titanium isopropoxide as second catalyst at 150° C. (seventh bar) being a replicate of the conditions of the sixth bar.

B in the figures relates to Bentonite. PGO in the figures relates to poly (glycerol oxalate) (polyester according to the present invention). For example, PGO+B in the figures refer to poly (glycerol oxalate) plus Bentonite (agricultural composition according to the present invention. Numbers after letters, for example B1, B2, B3, PGO1, PGO2, PGO3, PGO+B1, PGO+B2 or PGO+B3 relate to the sample number within a triplicate.

### EXAMPLES

The following examples illustrate some specific example embodiments of the present disclosure. These examples represent specific approaches found to function well in the practice of the application, and thus can be considered to constitute examples of modes for its practice. However, those of skill in the art should, in light of the present disclosure, appreciate that many changes can be made in the specific embodiments that are disclosed without departing from the spirit and scope of the application.

If not otherwise stated, the polyester according to the present invention referred to in the following examples is made from CO₂ converted into a (COOH)₂, which (COOH)₂ is converted into an oxalic acid ester which oxalic acid ester is combined with glycerine to obtain a polyester in particular hydrogel having a structure according to Formula I, II, III, or IV:

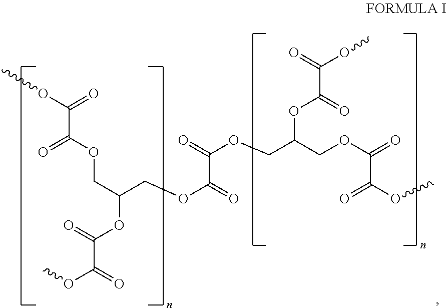

with n ranging from 1 to 5250,

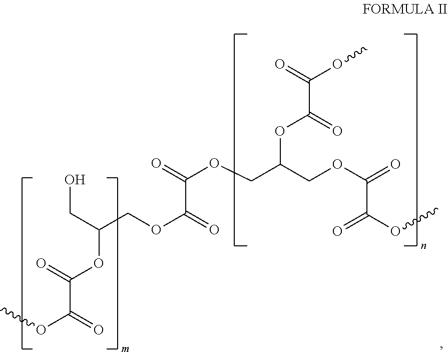

with n and m each ranging independently from 1 to 5250 or

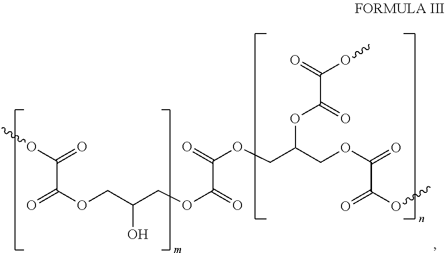

with n and m each ranging independently from 1 to 5250, or

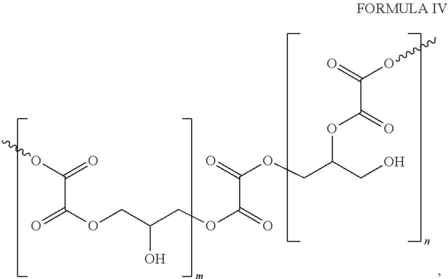

with n and m each ranging independently from 1 to 5250.

If not otherwise stated, the commercially available hydrogels, in particular Miracle Gro, Terra Sorb, Soil Moist and Diaper Polymer, as referred to herein and used as comparison to the polyester and agricultural composition according to the present invention do not comprise polyesters.

### Example 1 Water Holding Capacity (WHC)/Water Absorption Experiments of Polyesters According to the Present Invention

### Method 1

The water holding capacity determined using method 1 is determined at one particular moment as follows:
submerse sample in water,
record wet mass,
dry sample and
record dry mass
calculate WHC according to formula below.

In detail method 1 was conducted as follows:

The experiments were done in triplicate: 1 g of polyester sample was submerged in 2.5 mL demineralized water, shaken for 15 minutes and rested for 1 h. Afterwards, the wet polyester sample mass was filtrated from the liquid and drained for 10 minutes. Then, the wet polyester sample mass was weighed. (optional: washing step by repeating the water addition and filtration). The wet polyester sample mass was dried in a vacuum oven at 30° C. to 40° C. overnight and the dry polyester sample mass was weighed.

The water holding capacity WHC was calculated according to the following equation:

$$WHC = \frac{Wet sample weight - oven dry sample weight}{Oven dry sample weight}$$

TABLE 2
WHC of samples produced with titanium
isopropoxide as second catalyst.

| wt. % | | | Standard | |
|---|---|---|---|---|
| catalyst | Temperature/° C. | WHC | deviation | Notes |
| 0.1 | 150 | 128% | 31% | |
| 0.5 | 120 | 14% | 4% | incl. washing steps |
| 0.5 | 150 | 143% | 48% | |
| 0.5 | 150 | 84% | 6% | replicate, incl. washing |
| | | | | steps |
| 0.5 | 180 | 152% | 63% | incl. washing steps |
| 1 | 150 | 50% | 30% | |
| 1 | 150 | 66% | 11% | replicate |

Table 2 shows that there is no big difference in the WHC of the polyesters according to the present invention using different reaction conditions.

TABLE 3
WHC of samples produced with p-toluenesulfonic
acid as second catalyst.

| wt. % | | | Standard | |
|---|---|---|---|---|
| catalyst | Temperature/° C. | WHC | deviation | Notes |
| 0.3 | 150 | 128% | 8% | |
| 0.3 | 180 | 115% | 87% | incl. washing steps |
| 1 | 150 | 57% | 21% | incl. washing steps |

First and second columns in tables 2 and 3 indicate preparation parameters, third and fourth column indicate the result of the WHC analysis and the last column indicates notes for variations in the preparation processes.

FIG. 12 shows the WHC comparison of samples synthesised with (a) p-toluenesulfonic acid (FIG. 12 left; table 3) and (b) titanium isopropoxide (FIG. 12 right; table 2) as catalysts at different reaction conditions, carried out in triplicate.

TABLE 4
Comparison of a polyester sample of the present
invention with commercially available hydrogels.

| | | | WHC/g H₂O g |
|---|---|---|---|
| Material | | WHC | dry wt⁻¹ |
| Polyester Sample | | 68% | 0.68 ± 0.04 |
| Bentonite + 5% Polyester sample | | 330% | 3.3 |
| Bentonite | | 450% | 4.49 |
| Soil | | 33% | 0.33 |
| Commercially | agricultural hydrogels | 450% | 4.49 |
| available | diaper hydrogels | 20,000% | 216 |
| hydrogels: | | | |

FIG. 13 shows the water holding capacity (WHC) for commercially available hydrogels (first four bars from left to right in FIG. 13), Bentonite (fifth bar) from left to right in FIG. 14), a polyester according to the present invention, in particular hydrogel (termed Shell gel, sixth bar from left to right in FIG. 13) and agricultural compositions according to the present invention comprising Bentonite and different amounts (5%, 10% and 20%) of the polyester according to the present invention (seventh to ninth bar from left to right in FIG. 13). Different letters indicate significant differences (p<0.05) between treatments in terms of the WHC, and no significant differences where letters are the same.

### Method 2: Diffusion Method for Determining WHC/Absorption

The Diffusion method relies on diffusion of water vapour to a sample and yields long-term exposure plots.

The water absorption experiments were performed in a closed desiccator wherein one beaker with faucet water and one beaker with various polyesters according to the present invention (hereinafter also called polyester sample (PE)) were placed. The water uptake is monitored by carefully weighing the polyester sample over a certain timeframe and by recording the mass difference. The vapour pressure of water in air at 20° C. was ˜0.02 bar, and the relative humidity in the desiccator was 20 to 30%.

The results are visualized as plots of mass difference vs. time FIGS. 3-11 and in table 1.

FIG. 4 shows the time dependence of the mass balance during water uptake of the sample at ambient temperature.

TABLE 1
Time dependent mass for different polyester samples,
prepared using different reaction conditions:

| Polyester | | |
|---|---|---|
| sample | reaction conditions | WHC |
| J1-A | SnCl₂ * 2 H₂O, | 185 wt. % uptake in 67 days |
| (Hydrogel | 160/130° C. | |
| No. 1) | | |
| J2-A | SnCl₂ * 2 H₂O, | 255 wt. % uptake in 67 days |
| (Hydrogel | 160/160° C. | |
| No. 2) | | |
| J3-A | H₂SO₄, 160/130° C. | 260 wt. % uptake in 67 days |
| (Hydrogel | | |
| No. 3) | | |

The reaction conditions given in table 1 refer to the second acid catalyst and the temperature used in step b) when combining the carboxylic acid ester with glycerine.

FIG. 5 shows the time dependence of the mass difference for poly (glycerol oxalate) samples (hydrogel No. 1 to 3) as listed in table 1 at ambient temperature.

FIG. 6 shows for sample 6 prepared with p-Tos [p-toluenesulfonic acid] as second acid catalyst a 150% water uptake per g (sample 6) after 60 days.

FIG. 7 left shows sample 14 prepared with Sb₂O₃ as second catalyst and diethyl oxalate as reactant (triangle), sample 21 prepared with H₂SO₄ as second catalyst and diethyl oxalate as reactant (diamond) and sample 26 prepared with SnCl₂ as second catalyst and dimethyl oxalate as reactant (rectangle). All take up 130% of their own weight in water after 30 days.

Small differences in water absorption among the polyester samples, in particular poly (glycerol oxalate)s were observed, irrespective of reaction conditions in terms of the catalyst, temperature, and pressure applied. (FIG. 6 and left in FIG. 7). Water uptake is 130-150% of the sample weight for sample 6 and 21.

Small differences were observed between polyester samples made under various conditions. However, if the polyester according to the present invention, in particular poly (glycerol oxalate) (rectangle, sample 21), is compared to the ethylene glycol derivative (diamond, sample 31) (FIG. 7 right), a faster increase of the mass of the polyester according to the present invention, in particular poly (glycerol oxalate), is observed indicating a faster absorption of water accompanied by a larger total amount of water taken up. Also lower WHC was observed when oxalic acid was used in step (c) as reactant for the polymerization instead of diethyl oxalate, and if the vacuum step was omitted (FIG. 8).

The WHC for sample 21 prepared with H₂SO₄ as second acid catalyst and mixed with soil (20 wt. % PGO and 80 wt. % soil) is shown in FIG. 9, left. It shows that 130% sample weight taken up in form of water after 30 days was measured.

FIG. 9 right shows sample 21 prepared with H₂SO₄ as second acid catalyst with 200 wt. % water at t=0. The maximum uptake of water for sample 21 was determined to a threshold at 475 to 480 wt. % of sample weight taken up in form of water after 4 months. The water holding was stable for 2 months at least.

A polyester hydrogel, was prepared from glycerine and catalytic Sb₂O₃ according to disclosed embodiments and then contacted with water to analyse water uptake dependence on time with the results being shown in FIGS. 3 and 4. The hydrogel was introduced to a water source for 2,800 hours and the mass of the hydrogel was measured periodically. As shown in FIG. 3, the water uptake is dependent on time. Water was absorbed and released periodically and was shown to correlate with temperature. FIG. 3 shows the time dependence of the mass difference during water uptake/release cycles at alternating temperatures of 25° C. and 35° C. The mass change (Δm) ranged from 0 to about 160% throughout the 2,700 hour time frame. Additionally, as shown in FIG. 4, a hydrogel was able to absorb enough water to increase its mass to almost three times the original mass over an 800 hour period.

FIG. 10 shows the reversibility of the water uptake of polyester samples 6 prepared with p-Tos as second acid catalyst and 14 prepared with Sb₂O₃ as second acid catalyst. The samples were subjected to alternating temperatures of 35° C. (no additional exposure to water to check water release) and room temperature (25° C.) (within an aqueous atmosphere to check the water uptake) over a certain timeframe (FIG. 10 left, ˜20 cycles of uptake release, and right, ˜20cycles of uptake release). For samples 6 and 14 the water uptake/release was reversible for at least 38 weeks.

### Example 2: Genetic Data of Soil with and without Addition of Different Hydrogels, Bentonite and a Polyester and an Agricultural Composition According to the Present Invention

DNA was purified from soil samples (using the MPBio FastDNA purification kit by the Promega Maxwell liquid handling robot) which had been incubated with different commercially available hydrogels, Bentonite and a polyester and an agricultural composition according to the present invention: Soil only, Bentonite, Poly (glyercol oxalate) (abbreviated as PGO, that means polyester according to the present invention), Poly (glyercol oxalate) combined with Bentonite (abbreviated PGO+B, that means agricultural composition), Miracle Gro, Soil Moist Gel, Terra Sorb and Diaper Polymer.

Basecalled DNA sequence reads were demultiplexed (split into their individual originating samples) and taxonomically classified using DIAMOND against the NCBI non-redundant database. Classifications were visualised in MEGAN.

The Soil Microbiome baseline was dominated by Acidobacteria, Proteobacteria and Terrabacteria.

### Alpha Diversity

The Alpha-diversity within the sample was measured using the Shannon-Weaver index and the class taxonomic level (FIG. 14).

There is a significant difference (P=0.0003), in particular decrease, between the diversity of the soil only community (first in FIG. 14 from left to right) and the soil community which was incubated with Poly(glycerol oxalate) (PGO; polyester according to the present invention) only (third in FIG. 14 from left to right) and PGO combined with bentonite (fourth in FIG. 14 from left to right).

The PGO and PGO combined with bentonite reduce the species richness and content per unit area of the soil, meaning that selected microorganism will thrive in the presence of PGO and PGO combined with bentonite. Advantageously, the bacteria population decreases (which would consume the PGO or PGO+B and release CO₂ back to atmosphere through their respiration process), while fungi population (no breathing, thus no CO₂ release) would decompose the PGO into decomposition products, which remain in the soil (thus sequestering carbon).

### Beta Diversity

The Beta-diversity between samples was measured using the Bray-Curtis dissimilarity method and the matrix visualised on a neighbor joining tree (FIG. 15). An increased distance is a measure of increased microbial diversity between those samples.

All samples are measured in triplicate which is tated by numbers 1, 2, 3. Further: MG=Miracle Grow, TS=Terra Sorb, DP=Diaper Polymer, SM=Soil Moist, B=Bentonite, PGO=poly(glycerol oxalate); polyester according to the present invention), PGO+B=poly(glycerol oxalate) combined with Bentonite; agricultural composition according to the invention.

The soil only (circles in FIG. 15) and bentonite samples (B, pentagons in FIG. 15) show minimal diversity between them, there is a small difference between the soil and Miracle Gro (MG, stars with four tips in FIG. 15) samples, further diversity changes are seen to the Terra Sorb (TS, rectangles in FIG. 15), Soil Moist (SM, triangles with tip upside in FIG. 15) and Diaper Polymer (DP, stars with five tips in FIG. 15). The greatest diversity change against the soil control is observed with Poly (glycerol oxalate) (PGO; Polyester according to the present invention) when incubated with bentonite (PGO+B; agricultural composition according to the present invention diamonds in FIG. 15) or without bentonite (PGO; triangles with tip downside in FIG. 15).

As Beta diversity refers to changes in microbial community composition (degree of community differentiation) in relation to the original soil environment, PGO and PGO+Bentonite change the microbial community in a profound way by letting survive the microorganisms capable of carbon sequestration.

### Fungal to Bacterial Ratio

An increased fungi to bacteria ratio is observed in the soil incubated with the Poly(glycerol oxalate) (PGO; Polyester according to the present invention, seventh to ninth marking in FIG. 16 from left to right) and Poly (glycerol oxalate) with Bentonite (PGO+B; agricultural composition according to the present invention, tenth to twelveth marking in FIG. 16 from left to right) indicating that PGO either inhibits bacteria or promotes fungal growth (FIG. 16). All samples are measured in triplicate which is tated by numbers 1, 2, 3. Further: PGO=poly(glycerol oxalate) (polyester according to the present invention), PGO+B=poly (glycerol oxalate)+Bentonite (agricultural composition according to the present invention).

### Identified Taxa

Observed abundance changes in:
Ascomycota—appearance of this fungal taxa in PGO incubated samples;
Proteobacteria—increase in abundance of proteobacteria in PGO incubated samples, driven by a rise specifically in Alphaproteobacteria;
Actinobacteria—increase in abundance in PGO incubated samples;
Thaumarchaeota—disappearance of this taxa in PGO incubated samples;
Acidobacteria—reduction in abundance in PGO samples;
Chloroflexi—reduction in abundance in PGO samples; and
Bacteroidetes—reduction in “PGO only”—incubations, no major difference in PGO+B (PGO with bentonite) samples compared to other hydrogels.

### Conclusions

Incubation with PGO significantly decreases the diversity of the microbiome. There is an increase in the fungal to bacterial ration in soil microbiomes incubated with PGO.

The diversity change is attributed to predominantly a reduction in Acidobacteria and Chloroflexi, and an increase in Proteobacteria and Ascomycota.

Advantageously, the bacteria population decreases (which would consume the PGO or PGO+B and release CO₂ back to atmosphere through their respiration process) while fungi population increases (no breathing, thus no CO₂ release) which would decompose the PGO into decomposition products, which remain in the soil (thus sequestering carbon).

### Example 3: Biological Degradation Tests of a Polyester and an Agricultural Composition According to the Present Invention, Bentonite and Commercially Available Hydrogels in Soil

### Test Method

A method typically used to assess decomposition rates mediated by microbial populations in soil involve lab-based assays with soil and samples maintained at constant temperature and moisture content over time. These sample incubations are assayed weekly for their soil respiration activity by measuring increases in CO₂ over short term closed incubations periods. For replicates (n=3 for each) of soil only (FIG. 17 g)) soil plus polyester according to the present invention (FIG. 17 d)) or agricultural composition according to the present invention (FIG. 18 e)) and soil plus Bentonite or commercially available hydrogels (FIG. 17 a), b), c), f), h)) combination degradation assays were setup in 50 mL falcon tubes and their water holding capacity (WHC) adjusted to 65% through the addition of deionised water. Each tube with soil received 8 g dry weight of soil, 1 wt. % of the soil dry weight as polyester or agricultural composition according to the present invention, Bentonite or commercially available hydrogel was added to the soils and tubes with hydrogel treatments. This 1 wt. % value was deemed to represent a typical agricultural hydrogel amendment rate. Once all the experimental tubes were setup and water was added, they were immediately assayed for soil respiration using a 3-hour-incubation-method.

Between weekly gas measurements, the tubes were stored at 20° C. with perforated parafilm covering the tubes to minimize soil water losses due to evaporation but still allow free air gas exchange. After each weekly gas flux measurement, the soils were re-wetted to their 65% WHC by addition of deionized water.

### Soil Gas Sampling and Flux Measurements

The soil gas flux measurements were performed weekly. This involved the use of Falcon tube lids with septa to allow for a gas sampling at times of 0, 1, 2, and 3 hours after the sample tube was capped. After the septa lid has been added the tube was flushed with CO₂ free air (zero-air) to provide a constant starting CO₂ concentration at time=0 h. The flushing of tubes was performed by inserting a needle running from a zero-air gas line into the septa, with another syringe needle inserted to allow incoming zero-air to flush the headspace in the 50 mL Falcon tube for 2 mins. After 2 mins of flushing the two needles were removed from the tube and transferred to the next sample to be flushed.

Whilst the next tube was being flushed the previous flushed tube headspace gas was sampled by adding 20 mL zero-air using an SGE gas tight syringe, followed by headspace mixing, and finally a 20 mL headspace sample was removed and added to a pre-evacuated 12 mL Exetainer. This process was repeated for each of the Falcon tubes, for time=0 h. After 1 hour another headspace sample was taken by adding another 20 mL of zero-air, followed by headspace mixing, and a 20 mL sample was taken and transferred to a pre-evacuated 12 mL Exetainer. This latter part was repeated after 2 and 3 hours. At the end of the incubation all lids were removed, the tube weights recorded, deionized water added where required to reach 65% WHC, and punctured parafilm added to the tops of the open tubes and re-incubated at 20° C. The Exetainer gas samples were then analysed for CO₂ concentration using a Picarro CRDS analyser.

### Biological Degradation Test Results

FIG. 17 shows the weekly soil respiration assays for a duration of 13 weeks. The incubations with PGO gel (polyester and agricultural composition according to the present invention, FIG. 17 d) and e)) had greater initial respiration rates than the other non-PGO gels (soil only (FIG. 17 g)) and Bentonite or commercially available hydrogels (FIG. 17 a), b), c), f) and h)), and temporal changes over time. The temporal increase and decrease in the respiration rates with the PGO gels are indicative of biodegradation of substrates into secondary metabolites that become available over time.

The non-PGO gel respiration rates were similar to that of the soil alone, indicating that there was limited biodegradation occurring over time with these incubations.

The diagrams in FIG. 17 show the temporal changes in soil and gel respiration rates for long term biodegradation incubation assays. Soil respiration rates were determined from the different soil and gel combinations assayed repeatedly over 13 weeks, with incubations maintained at 20° C. and 65% WHC throughout. The temporal dynamics are indicative of biodegradation products becoming available as microbially mediated mineralization proceeds. The weekly soil respiration assays from FIG. 17 were used to calculate a cumulative CO₂ loss over the entire 13-week period, these results are shown in FIG. 18. The cumulative flux of CO₂ from the PGO gels (polyester and agricultural composition according to the present invention, fourth bar and fifth bar from left to right in FIG. 18) was significantly greater than that from the non-PGO gels (Bentonite and commercially available hydrogels, first, second, third, sixth and seventh bar from left to right in FIG. 18), confirming that the PGO gels were more biodegradable than the other commercial gels. The non-PGO gels have negative cumulative changes in efflux because they could be inhibiting soil respiration and having negative priming effects on soil carbon cycling.

The diagram in FIG. 18 shows the cumulative gel biodegradation rates calculated from 13weeks of soil lab incubations at 65% WHC and 20° C. Results are expressed on a dry weight of gel plus soil basis after subtracting soil respiration rates from the gel plus soil data. Different letters indicate significant differences (p<0.05) between treatments in terms of cumulative change in CO₂ flux, and no significant differences where letters are the same based on pairwise t-tests.

### Example 4: Hydrostability Data as Indicator for Degradation and Therefore as Quality Differentiator for the Quality of the Polyesters According to the Present Invention

Agricultural polyesters, in particular hydrogels, are polymers that can retain high amounts of water. The polyester, in particular hydrogel, can be applied to the soil of crop plantations in systems with limited or intense/frequent rainfalls. The stability of the polyester, in particular hydrogel, in aqueous environments can indicate the durability of the polyester, in particular hydrogel. The hydrostability was determined by measuring the weight loss (wt %) of the dry polyester, in particular hydrogel, over the time span of up to one month at room temperature after contact with water at room temperature.

Exemplary hydrostability tests for p-toluenesulfonic acid (p-Tos) and titanium isopropoxide catalyzed polyesters according to the present invention were carried out.

### Chemicals

Polyester, in particular hydrogel, samples according to the present invention
Demineralized water

### Equipment

30 mL centrifuge tubes
Glass cover plate/weighing paper
Centrifuge
Volumetric cylinder

### Comparison of the Hydrostability of Differently Synthesised Polyesters According to the Present Invention

Degradation of polyesters according to the present invention can be characterised based on their stability against hydrolysis in the presence of water.

Weighed polyester samples are submerged in 2.5 mL water and shaken for 15 min. In an optional washing step, the water submersion and shaking is repeated twice. After resting overnight, the wet polyester mass is separated from the liquid with filter paper in a funnel on gravity over 10 min. The polyester samples are dried in a vacuum oven overnight at 30 to 40° C. and weighed again. The test is carried out in triplicates. By subtracting the dry polyester sample mass from the initial polyester sample mass, and dividing by the initial polyester sample mass, the remaining polyester sample mass ratio can be used to characterise the stability against hydrolysis degradation. The polyester sample mass ratio is calculated using the following formula:

$$Δ m = \frac{initial polyester mass ( g ) - dry polyester mass ( g )}{initial polyester mass ( g )}$$

The polyester samples synthesised with titanium isopropoxide and p-toluenesulfonic acid are stable in water overnight. There are no large differences between polyester samples synthesised at different reaction conditions.

After 24 h it was checked if the product has dissolved. If not, the above-mentioned steps should be repeated.

FIG. 20: Hydrostability of polyester samples according to the present invention synthesised with (a) p-toluenesulfonic acid (p-Tos) (FIG. 20 left), or (b) titanium isopropoxide (TTIP) (FIG. 20 right) as catalyst at different reaction conditions. The mass difference is the polyester sample amount that is not dissolved by water overnight at room temperature, relative to the initial polyester sample mass. The test was carried out in triplicate.

It is understood that the listed components for each unit are for illustration purposes only, and this is not intended to limit the scope of the application. A specific combination of these or other components or units can be configured in such a composition or method for the intended use based on the teachings in the application.

Persons skilled in the art may make various changes in the shape, size, number, separation characteristic, and/or arrangement of parts without departing from the scope of the instant disclosure. Each disclosed component, system, and process step may be performed in association with any other disclosed component, system, or process step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. Where desired, some embodiments of the disclosure may be practiced to the exclusion of other embodiments.

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55,but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/− about 10%, depicted value +/− about 50%, depicted value +/− about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

The Claims and the dependencies therein, by way of this reference, form an integral part of the present disclosure.

1. A method of sequestering carbon dioxide, the method comprising the steps of:
(a) capturing carbon dioxide from an industrial gaseous waste stream and/or the atmosphere;
(b) converting a CO2 from the CO2 gas stream into a (COOH)2; and
(c) combining the (COOH)2, a mono-alcohol (X-OH), preferably CH3CH2OH, and a first acid catalyst comprising a H2SO4 at a temperature ranging from about 80° C. to about 100° C. and under atmospheric pressure to produce an ester comprising a (COOX)2 and preferably (COOEt)2;
(d) the ester obtained in step (c) is reacted with a polyol, preferably glycerine to form a polyester, preferably the polyester is a hydrogel.

2. A method according to claim 1 wherein the polyester obtained in step (d) is a hydrogel and the method further comprises the step of:
(e) mixing the hydrogel with a soil and optionally nutrients, to obtain a soil-hydrogel mixture.

3. A method according to claim 1 wherein the method further comprises the step of: (f) providing the polyester, preferably hydrogel, or the hydrogel-soil mixture to a ground surface, preferably of agricultural land, forest ground or a field.

4. A method according to claim 1 wherein one or more species of fungi are added after step (d).

5. A method according to claim 1 wherein the hydrogel or hydrogel-soil mixture is shaped into particles.

6. A method according to claim 1 wherein the hydrogel comprises polyglycerol oxalate.

7. (canceled)

8. (canceled)

9. (canceled)

10. (canceled)

11. (canceled)
